(19) United States
(12) Patent Application Publication
(10) Pub. No.: US 2025/0000957 A1
(43) Pub. Date: Jan. 2, 2025

(54) COMPOSITIONS AND METHODS FOR TREATING PHENYLKETONURIA

(71) Applicant: Arcturus Therapeutics, Inc., San Diego, CA (US)

(72) Inventors: Ramon DIAZ TRELLES, San Diego, CA (US); Kiyoshi TACHIKAWA, San Diego, CA (US); Padmanabh CHIVUKULA, San Diego, CA (US)

(21) Appl. No.: 18/690,408

(22) PCT Filed: Sep. 15, 2022
(86) PCT No.: PCT/US22/76482

§ 371 Date: Mar. 8, 2024

Related U.S. Application Data
(60) Provisional application No. 63/244,887, filed on Sep. 16, 2021.

(51) Int. Cl. A61K 38/51; A61K 48/00; A61P 3/00; C12N 9/88; C12N 15/88

(52) U.S. Cl. CPC A61K 38/51; A61K 48/0066; A61P 3/00; C12N 9/88; C12N 15/88; C12Y 403/01024

(57) ABSTRACT

Phenylketonuria (PKU) is caused by a mutation in the phenylalanine hydroxylase (P AH) gene in the liver, Provided herein are compositions and methods for treating phenylketonuria (PKU) and related disorders. Polynucleotides for expressing bacterial and plant-derived phenylalanine ammonia lyase (PAL) are provided herein. Also provided herein are methods of treating PKU and related disorders that include administration of polynucleotides encoding PAL proteins.

### CROSS REFERENCE TO RELATED APPLICATION

This application claims priority to U.S. Provisional Application No. 63/244,887, filed Sep. 16, 2021, which is incorporated herein by reference in its entirety.

### TECHNICAL FIELD

The present disclosure relates generally to therapeutics and biopharmaceuticals for the treatment of metabolic disorders, and more specifically to translatable molecules for the treatment of phenylketonuria.

### REFERENCE TO SEQUENCE LISTING

The instant application contains a Sequence Listing, which has been submitted electronically in ASCII format and is hereby incorporated by reference in its entirety. Said ASCII copy, created on Sep. 13, 2022 is named “049386-545001WO_SL” and is 86,265 bytes in size.

### BACKGROUND

Phenylketonuria (PKU) is an inherited autosomal recessive metabolic disorder and one of the most common inborn errors of metabolism, affecting around 1 in 12,000 live births (1). PKU is caused by a mutation in the phenylalanine hydroxylase (PAH) gene in the liver, which facilitates the catabolism of phenylalanine (Phe) by conversion of Phe to tyrosine (Tyr). Without a functional copy of PAH, Phe levels in the blood and tissues rise, resulting in potentially life-threatening damage to the central nervous system, mental retardation, and other neurological deficits (2). The limited available treatment options for PKU require adherence to a strict low protein PKU diet that represents a significant obstacle for patient compliance and can result in growth retardation due to nutritional deficiencies (3).

Two FDA approved therapeutics are available for treatment of PKU. Sapropterin dihydrochloride (Kuvan®) (4, 5) is a synthetic form of tetrahydrobiopterin (BH4), the natural cofactor for the enzyme PAH. Because Sapropterin relies on activation of any residual functional PAH enzyme, it is only effective in a subset of patients and must be used in conjunction with a strict PKU diet (5, 6). Pegvaliase (Palynziq®) is a pegylated form of Anabaena variabilis derived phenylalanine ammonia lyase (avPAL) that catalyzes the degradation of Phe to ammonia and trans-cinnamic acid (tCA). Pegylation serves to reduce the immunogenicity of the bacterial PAL protein. However, almost all patients (93.5%) experienced hypersensitivity adverse events (HAE), and a significant proportion of patients (4.6%) suffered anaphylaxis during phase 3 trials, leading to restricted FDA approval under a Risk Evaluation and Mitigation Strategy (7, 8). Patients taking Pegvaliase must titrate up to the full dose over a 12-month period, are required to carry auto-injectable adrenaline (epinephrine), and prophylactic use of H1 and H2 blocking antihistamines prior to initiating treatment is recommended (9).

In summary, of two approved drugs that are available for treatment of PKU, one must be used in conjunction with a strict PKU diet while the other has serious immunological side effects. Thus, there exists a need for new therapeutics for the treatment of PKU and related disorders.

### SUMMARY

The present disclosure is based on the seminal discovery that phenylalanine ammonia lyase (PAL) expressed from mRNA delivered via lipid nanoparticles (LNPs) is biologically active and reduces phenylalanine levels in vivo in the absence of functional phenylalanine hydroxylase (PAH).

Provided herein, in some embodiments, are polynucleotides for expressing bacterial phenylalanine ammonia lyase (PAL) or a fragment thereof, wherein the polynucleotides include natural and chemically modified nucleotides, and wherein the polynucleotides are expressible to provide bacterial PAL or a fragment thereof having PAL activity. In one aspect, the bacterial PAL is Anabaena PAL. In another aspect, the bacterial PAL is Anabaena variabilis PAL. In yet another aspect, the bacterial PAL is a wild-type bacterial PAL or a mutant bacterial PAL. In a further aspect, polynucleotides for expressing wild-type or mutant bacterial PAL include a codon-optimized coding region encoding the wild-type or mutant bacterial PAL as compared to a wild-type or reference coding region encoding the wild-type or mutant bacterial PAL. In some aspects, the mutant bacterial PAL includes a mutation at C503, C565, or both C503 and C565. In some aspects, the mutant bacterial PAL includes a mutation selected from C503S, C565S, or both C503S and C565S. In other aspects, the coding region encoding the bacterial PAL includes a sequence having at least 80% identity to a sequence selected from SEQ ID NOs:1-4.

Also provided herein, in some embodiments, are polynucleotides for expressing plant phenylalanine ammonia lyase (PAL) or a fragment thereof, wherein the polynucleotides include natural and chemically modified nucleotides, and wherein the polynucleotides are expressible to provide plant PAL or a fragment thereof having PAL activity. In some aspects, the plant PAL is an Arabidopsis PAL, a Solanum PAL, or a Nicotiana PAL. In one aspect, the plant PAL is an Arabidopsis PAL. In another aspect, the plant PAL is an Arabidopsis thaliana PAL. In yet another aspect, the plant PAL is a Solanum PAL. In a further aspect, the plant PAL is a Solanum lycopersicum PAL. In one aspect, the plant PAL is a Nicotiana PAL. In another aspect, the plant PAL is a Nicotiana tabacum PAL. In some aspects, the plant PAL is a wild-type plant PAL or a mutant plant PAL. In other aspects, polynucleotides for expressing wild-type or mutant plant PAL provided herein include a codon-optimized coding region encoding the wild-type or mutant plant PAL as compared to a wild-type or reference coding region encoding the wild-type or mutant plant PAL. In further aspects, the coding region encoding the plant PAL includes a sequence having at least 80% identity to a sequence selected from SEQ ID NOs:5-7.

In some aspects, polynucleotides provided herein for expressing bacterial or plant PAL further include a 5′ UTR. In one aspect, the 5′ UTR includes a sequence having at least 80% identity to a sequence of SEQ ID NO:8. In other aspects, polynucleotides provided herein for expressing bacterial or plant PAL further include a 3′ UTR. In one aspect, the 3′ UTR includes a sequence having at least 80% identity to a sequence of SEQ ID NO:9. In further aspects, polynucleotides provided herein for expressing bacterial or plant PAL further include a 3′ poly(A) sequence. In one aspect, the poly(A) sequence includes about 100 nucleotides.

In some aspects, polynucleotides provided herein for expressing bacterial or plant PAL are RNA molecules that include U instead of T. In one aspect, RNA molecules provided herein are mRNA molecules. In another aspect, RNA molecules provided herein are self-replicating RNA molecules. In some aspects, RNA molecules provided herein further include a 5′ cap. In some aspects, the 5′ cap has a Cap 1 structure, a Cap 1 (m⁶A) structure, a Cap 2 structure, or a Cap 0 structure. In other aspects, RNA molecules provided herein include chemically modified nucleotides that include chemically modified nucleosides selected from 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine, 6-O-methylguanosine, and any combination thereof. In one aspect, the chemically modified nucleosides are N¹-methylpseudouridines. In another aspect, the chemically modified nucleosides are 5-methoxyuridines. In some aspects, the chemically modified nucleotides include 1-100% of the nucleotides that can be chemically modified. In other aspects, the chemically modified nucleotides include 50-100% of the nucleotides that can be chemically modified.

Provided herein, in some embodiments, are DNA molecules encoding polynucleotides provided herein. In one aspect, DNA molecules provided herein include a promoter. In one aspect, the promoter is located 5′ of the 5′ UTR. In another aspect, the promoter is a T7 promoter, a T3 promoter, or an SP6 promoter. In yet another aspect, the promoter is an RNA polymerase II promoter.

Provided herein, in some embodiments, are compositions that include a polynucleotide provided herein and a pharmaceutically acceptable carrier. In one aspect, the pharmaceutically acceptable carrier includes a lipid formulation. In another aspect, the lipid formulation is selected from a transfection reagent, a lipoplex, a liposome, a lipid nanoparticle, a polymer-based carrier, an exosome, a lamellar body, a micelle, and an emulsion. In yet another aspect, the lipid formulation is a liposome selected from a cationic liposome, a nanoliposome, a proteoliposome, a unilamellar liposome, a multilamellar liposome, a ceramide-containing nanoliposome, and a multivesicular liposome. In a further aspect, the lipid formulation is a lipid nanoparticle. In one aspect, the lipid formulation or lipid nanoparticle encapsulates polynucleotides provided herein. In some aspects, the lipid formulation includes a cationic lipid. In some aspects, the cationic lipid is an ionizable cationic lipid. In other aspects, the lipid formulation further includes at least one other lipid selected from the group consisting of anionic lipids, zwitterionic lipids, neutral lipids, steroids, polymer conjugated lipids, phospholipids, glycolipids, and combinations thereof.

Provided herein, in some embodiments, are methods for ameliorating, preventing, delaying onset, or treating a disease or condition associated with phenylketonuria, phenylalanine hydroxylase (PAH) deficiency, decreased metabolism of phenylalanine, or increased levels of phenylalanine in a subject in need thereof comprising: administering to the subject a polynucleotide or a composition provided herein. In one aspect, the administering increases expression of the bacterial or plant PAL protein or a fragment thereof in the liver, serum, plasma, kidney, heart, muscle, brain, cerebrospinal fluid, lymph nodes, or any combination thereof, as compared with administering a control polynucleotide or a control composition or vehicle. In another aspect, the administering decreases blood phenylalanine levels, increases blood trans-cinnamic acid (tCA) levels, increases blood hippurate (HA) levels, or any combination thereof, as compared with administering a control polynucleotide or a control composition or vehicle. In a further aspect, the administration is intravenous, subcutaneous, intradermal, transdermal, intranasal, oral, sublingual, intraperitoneal, intramuscular, topical, or by a pulmonary route. In yet a further aspect, the administering includes a therapeutically effective dose of from 0.01 mg/kg to 10 mg/kg.

### BRIEF DESCRIPTION OF THE DRAWINGS

FIGS. 1A-1D show efficacy of bacterial avPAL expression in vitro and in a PKU mouse model. (1A) In vitro expression levels of avPAL variants and confirmation of biological activity by the presence of the Phe metabolite tCA. (1B) Dose-dependent expression of avPAL protein in the PKU mouse model. (1C, 1D) Confirmation of biological activity of avPAL protein in a PKU mouse model as seen by reduction of serum Phe levels (1C) and increase in the level of the Phe metabolite HA (1D).

FIGS. 2A-2E show expression and function of plant-based PAL mRNA in vitro and in vivo. (2A) Comparison of in vitro expression of bacterial and plant-derived PAL proteins. Plant-derived PAL has a higher molecular weight (MW) than bacterial PAL, accounting for the difference in band position. (2B) Levels of Phe and (2C) levels of the Phe metabolite tCA for each of the four PAL protein variants in vitro. Blood serum levels of (2D) Phe, and (2E) the Phe metabolite HA in PKU mice after transfection with each of the four PAL protein variants.

### DETAILED DESCRIPTION

The present disclosure relates to compositions and methods for the treatment of PKU and related disorders. In particular, composition and methods of the disclosure provide bacterial and plant-derived PAL proteins expressed from mRNAs, delivery of mRNAs encoding PAL proteins using lipid nanoparticles (LNPs), and treatment of disorders resulting from phenylalanine hydroxylase (PAH) deficiency.

### Polynucleotides for Expressing Phenylalanine Ammonia Lyase (PAL)

Provided herein, in some embodiments, are polynucleotides for expressing bacterial phenylalanine ammonia lyase (PAL) or a fragment thereof. Accordingly, in some embodiments, polynucleotides provided herein are expressible to provide bacterial PAL or a fragment thereof having PAL activity.

Also provided herein, in some embodiments, are polynucleotides for expressing plant phenylalanine ammonia lyase (PAL) or a fragment thereof. Accordingly, in some embodiments, polynucleotides provided herein are expressible to provide plant PAL or a fragment thereof having PAL activity.

As used herein, the term “fragment,” when referring to a protein or nucleic acid, for example, means any shorter sequence than the full-length protein or nucleic acid. Accordingly, any sequence of a nucleic acid or protein other than the full-length nucleic acid or protein sequence can be a fragment. As used herein, the term “polynucleotide” refers to a molecule that includes at least two nucleotide monomers. The terms “polynucleotide,” “nucleic acid,” and “nucleic acid molecule” can be used interchangeably, unless context clearly indicated otherwise. Accordingly, as used herein, “polynucleotide,” “nucleic acid,” or “nucleic acid molecule” can refer to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA (cfDNA), mitochondrial DNA, chloroplast DNA, viral DNA, mRNA, tRNA, rRNA, long non-coding RNA, siRNA, micro RNA (miRNA or miR), hnRNA, and viral RNA. Exemplary nucleic analogues include peptide nucleic acid, morpholino- and locked nucleic acid, glycol nucleic acid, and threose nucleic acid. As used herein, the terms “polynucleotide,” “nucleic acid,” and “nucleic acid molecule” are meant to include fragments of polynucleotides, nucleic acids, or nucleic acid molecules as well as any full-length or non-fragmented polynucleotide, nucleic acid, or nucleic acid molecule, for example.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

Polynucleotides for expressing bacterial PAL provided herein can include natural and chemically modified nucleotides. Any natural nucleotide and any chemically modified nucleotide can be included in polynucleotides provided herein. Exemplary nucleobases of nucleotides include guanine (G), adenine (A), cytosine (C), thymine (T), uracil (U), and inosine (I). It will be appreciated that T is present in DNA, while U is present in RNA. In the examples of modified or chemically modified nucleotides provided herein, an alkyl, cycloalkyl, or phenyl substituent may be unsubstituted, or further substituted with one or more alkyl, halo, haloalkyl, amino, or nitro substituents. As used herein, the terms “chemically modified nucleotide” and “modified nucleotide” can be used interchangeably, unless context clearly indicates otherwise. Chemically modified nucleotides can include non-natural nucleotides.

Examples of modified or chemically modified nucleotides or nucleosides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴,N⁴-dialkylcytidines.

Examples of modified or chemically modified nucleotides or nucleosides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴,N⁴-dimethylcytidine.

Examples of modified or chemically modified nucleotides or nucleosides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically modified nucleotides or nucleosides include 5-hydroxyuridine, 5-methyluridine, 5-hydroxymethyluridine, 5-carboxyuridine, 5-carboxymethylesteruridine, 5-formyluridine, 5-methoxyuridine, 5-propynyluridine, 5-bromouridine, 5-fluorouridine, 5-iodouridine, 2-thiouridine, and 6-methyluridine.

Examples of modified or chemically modified nucleotides or nucleosides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically modified nucleotides or nucleosides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenine, 2-methoxy-adenine, alpha-thio-adenosine, 2′-O-methyl-adenosine, N⁶,2′-O-dimethyl-adenosine, N⁶,N⁶,2′-O-trimethyl-adenosine, 1,2′-O-dimethyl-adenosine, 2′-O-ribosyladenosine, 2-amino-N⁶-methyl-purine, 1-thio-adenosine, 2′-F-ara-adenosine, 2′-F-adenosine, 2′-OH-ara-adenosine, and N⁶-(19-amino-pentaoxanonadecyl)-adenosine.

Examples of modified or chemically modified nucleotides or nucleosides include N¹-alkylguanosines, N²-alkylguanosines, thienoguanosines, 7-deazaguanosines, 8-oxoguanosines, 8-bromoguanosines, O⁶-alkylguanosines, xanthosines, inosines, and N¹-alkylinosines.

Examples of modified or chemically modified nucleotides or nucleosides include N¹-methylguanosine, N²-methylguanosine, thienoguanosine, 7-deazaguanosine, 8-oxoguanosine, 8-bromoguanosine, O⁶-methylguanosine, xanthosine, inosine, and N¹-methylinosine.

Examples of modified or chemically modified nucleotides or nucleosides include pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine, N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

Examples of nucleic acid monomers include modified and chemically modified nucleotides, including any such nucleotides known in the art.

Examples of modified and chemically modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically modified nucleotide monomers include 3′-end stabilized nucleotides, 3′-glyceryl nucleotides, 3′-inverted abasic nucleotides, and 3′-inverted thymidine.

Examples of modified and chemically modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides.

Examples of modified and chemically modified nucleotide monomers include 2′,4′-constrained 2′-O-methoxyethyl (cMOE) and 2′-O-Ethyl (cEt) modified DNA monomers.

Examples of modified and chemically modified nucleotide monomers include 2′-amino nucleotides, 2′-O-amino nucleotides, 2′-C-allyl nucleotides, and 2′-O-allyl nucleotides.

Examples of modified and chemically modified nucleotide monomers include N⁶-methyladenosine nucleotides.

Examples of modified and chemically modified nucleotide monomers include nucleotide monomers with modified bases or modified bases of nucleosides, such as 5-(3-amino)propyluridine, 5-(2-mercapto)ethyluridine, 5-bromouridine; 8-bromoguanosine, or 7-deazaadenosine.

Examples of modified and chemically modified nucleotide monomers include 2′-O-aminopropyl substituted nucleotides.

Examples of modified and chemically modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Some examples of modified nucleotides are given in Saenger, Principles of Nucleic Acid Structure, Springer-Verlag, 1984.

Example of base modifications described above can be combined with additional modifications of nucleoside or nucleotide structure, including sugar modifications and linkage modifications.

Certain modified or chemically modified nucleotide monomers may be found in nature.

Polynucleotides provided herein can also include one or more unlocked nucleic acid (UNA) monomers. UNA monomers are small organic molecules based on a propane-1,2,3-tri-yl-trisoxy structure as shown below:

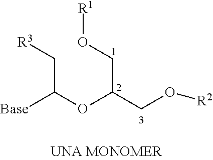

where R¹ and R² are H, and R¹ and R² can be phosphodiester linkages, Base can be a nucleobase, and R³ is a functional group described below.

In another view, the UNA monomer main atoms can be drawn in IUPAC notation as follows:

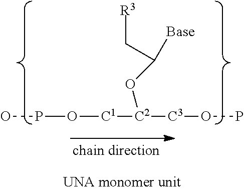

where the direction of progress of the oligomer or polymer chain is from the 1-end to the 3-end of the propane residue. Examples of a nucleobase include uracil, thymine, cytosine, 5-methylcytosine, adenine, guanine, inosine, and natural and non-natural nucleobase analogues. Further examples of a nucleobase include pseudouracil, 1-methylpseudouracil (m1Ψ), i.e., N¹-methylpseudouracil, and 5-methoxyuracil.

Accordingly, polynucleotides provided herein can include combinations of UNA monomers with certain natural nucleotides, non-natural nucleotides, modified nucleotides, or chemically modified nucleotides. In general, a UNA monomer can be an internal linker monomer in an oligomer or polymer. An internal UNA monomer in an oligomer or polymer is flanked by other monomers on both sides. A UNA monomer can participate in base pairing when the oligomer or polymer forms a complex or duplex, for example, and there are other monomers with nucleobases in the complex or duplex.

Examples of UNA monomers as internal monomers flanked at both the propane-1-yl position and the propane-3-yl position, where R³ is —OH, are shown below.

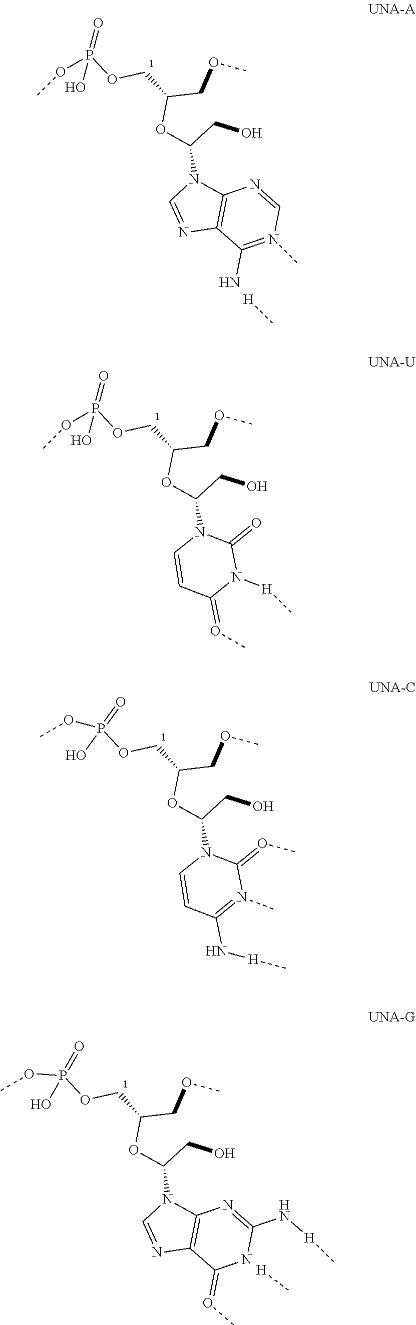

A UNA monomer can be a terminal monomer of an oligomer or polymer, where the UNA monomer is attached to only one monomer at either the propane-1-yl position or the propane-3-yl position. Because the UNA monomers are flexible organic structures, unlike nucleotides, the terminal UNA monomer can be a flexible terminator for the oligomer or polymer.

Examples UNA monomers as terminal monomers attached at the propane-3-yl position are shown below.

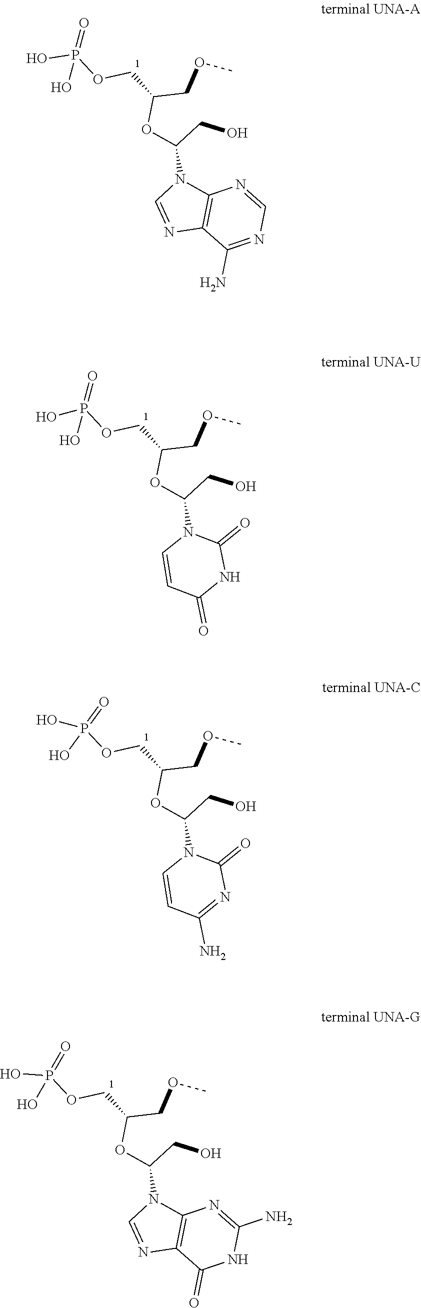

Because a UNA monomer can be a flexible molecule, a UNA monomer as a terminal monomer can assume widely differing conformations. An example of an energy minimized UNA monomer conformation as a terminal monomer attached at the propane-3-yl position is shown below.

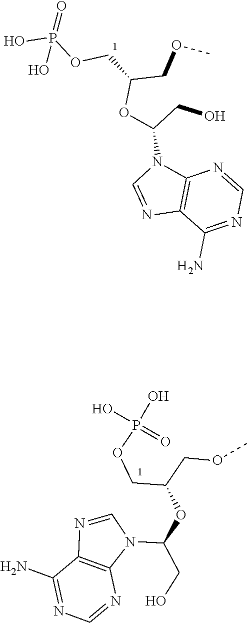

UNA-A terminal forms: the dashed bond shows the propane-3-yl attachment

Among other things, the structure of the UNA monomer allows it to be attached to naturally occurring nucleotides. A UNA oligomer or polymer can be a chain composed of UNA monomers, as well as various nucleotides that may be based on naturally occurring nucleosides.

In some aspects, the functional group R³ of a UNA monomer can be —OR⁴, —SR⁴, —NR⁴₂, —NH(C═O)R⁴, morpholino, morpholin-1-yl, piperazin-1-yl, or 4-alkanoyl-piperazin-1-yl, where R⁴ is the same or different for each occurrence, and can be H, alkyl, a cholesterol, a lipid molecule, a polyamine, an amino acid, or a polypeptide.

Generally, UNA monomers are not naturally occurring, modified naturally occurring, or chemically modified naturally occurring nucleotides, nucleosides, or monomers.

A UNA oligomer or polymer provided herein can be a synthetic chain molecule.

As shown above, a UNA monomer can be UNA-A (designated Ã), UNA-U (designated Ũ), UNA-C (designated Č), and UNA-G (designated Ǧ).

Designations that may be used herein include mA, mG, mC, and mU, which refer to the 2′-O-Methyl modified ribonucleotides.

Designations that may be used herein include dT, which refers to a 2′-deoxy T nucleotide.

As used herein, in the context of oligomer sequences, the symbol N can represent any natural nucleotide monomer, or any modified nucleotide monomer.

As used herein, in the context of oligomer or polymer sequences, the symbol Q may be used to represent a non-natural, modified, or chemically modified nucleotide monomer.

As used herein, in the context of oligomer or polymer sequences, the symbol X may be used to represent a UNA monomer.

In some aspects, polynucleotides provided herein have a structure of Formula I.

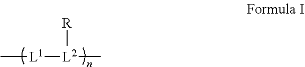

wherein L¹ is a linkage, n is from 200 to 12,000, and for each occurrence L² is a UNA linker group having the formula -C¹-C²-C³-, where R is attached to C² and has the formula —OCH(CH₂R³)R⁵, where R³ is —OR⁴, —SR⁴, —NR⁴₂, —NH(C═O)R⁴, morpholino, morpholin-1-yl, piperazin-1-yl, or 4-alkanoyl-piperazin-1-yl, where R⁴ is the same or different for each occurrence and is H, alkyl, a cholesterol, a lipid molecule, a polyamine, an amino acid, or a polypeptide, and where R⁵ is a nucleobase, or L²(R) is a sugar such as a ribose and R is a nucleobase, or L² is a modified sugar such as a modified ribose and R is a nucleobase. In certain embodiments, a nucleobase can be a modified nucleobase. L¹ can be a phosphodiester linkage.

In some aspects, polynucleotides provided herein can have any number of phosphorothioate intermonomer linkages in any intermonomer location.

In some aspects, any one or more of the intermonomer linkages of polynucleotides provided herein can be a phosphodiester, a phosphorothioate including dithioates, a chiral phosphorothioate, and other chemically modified forms.

When a oligomer, polymer, or polynucleotide provided herein terminates in a UNA monomer, the terminal position has a 1-end, or the terminal position has a 3-end, according to the positional numbering shown above.

### PAL Proteins

In some aspects, PAL proteins expressed from polynucleotides provided herein are derived from prokaryotes. In some aspects, PAL proteins expressed from polynucleotides provided herein are bacterial PAL proteins. Polynucleotides provided herein can express PAL proteins from any bacterium or bacterial strain, including cyanobacteria and Gram negative bacteria such as members of the Enterobacteriaceae family, for example. In some aspects, bacterial PAL provided herein is an Anabaena PAL, a Nostoc PAL, a Streptomyces PAL, an Anacystis PAL, a Brevibacillus PAL, a Planctomyces PAL, or a Photorabdus PAL. In other aspects, bacterial PAL provided herein is an Anabaena variabilis PAL (AvPAL), a Nostoc punctiforme PAL, a Streptomyces maritimus PAL, a Streptomyces verticillatus PAL, a Streptomyces rimosus PAL, a Anacystis nidulans PAL, a Brevibacillus laterosporus PAL, a Planctomyces brasiliensis, or a Photorabdus luminescens PAL. In still other aspects, the bacterial PAL is an Anabaena variabilis PAL (AvPAL, Q3M5Z3). In still other aspects, the PAL is from a eukaryotic organism that can be single-celled or multi-celled, such as a slime mold. Accordingly, in some aspects, the PAL is Dictyostelium PAL. In other aspects, the PAL is Dictyostelium discoideum PAL. Additional sources of PAL and/or organisms with PAL enzyme activity can be found in Weise, N. J., Ahmed, S. T., Parmeggiani, F. et al. Zymophore identification enables the discovery of novel phenylalanine ammonia lyase enzymes. Sci Rep 7, 13691 (2017). doi.org/10.1038/s41598-017-13990-0, which is incorporated herein by reference in its entirety.

In some aspects, PAL proteins expressed from polynucleotides provided herein are derived from eukaryotes, such as plants, yeast, and other fungi. Exemplary PAL proteins include Q9ATN7 Agastache rugosa; 093967 Amanita muscaria (Fly agaric); P35510, P45724, P45725, Q9SS45, Q8RWP4 Arabidopsis thaliana (Mouse-ear cress); Q6ST23 Bambusa oldhamii (Giant timber bamboo); Q42609 Brom Populus balsamifera subsp. trichocarpaxPopulus deltoides headia finlaysoniana (Orchid); P45726 Camellia sinensis (Tea); Q9MAX1 Catharanthus roseus (Rosy periwinkle; Madagascar periwinkle); Q9SMK9 Cicer arietinum (Chickpea); Q9XFX5, Q9XFX6 Citrus clementinaxCitrus reticulate; Q42667 Citrus limon (Lemon); Q8H6V9, Q8H6W0 Coffea canephora (Robusta coffee); Q852S1, 023865 Daucus carota (Carrot); 023924 Digitalis lanata (Foxglove); P27991 Glycine max (Soybean); 004058 Helianthus annuus (Common sunflower); P14166, Q42858 Ipomoea batatas (Sweet potato); Q8GZR8, Q8W2E4 Lactuca sativa (Garden lettuce); 049835, 049836 Lithospermum erythrorhizon; P35512 Malus domestica (Apple; Malus sylvestris); Q94C45, Q94F89 Manihot esculenta (Cassava; Manioc); P27990 Medicago sativa (Alfalfa); P25872, P35513, P45733 Nicotiana tabacum (Common tobacco); Q6T1C9 Quercus suber (Cork oak); P14717, P53443, Q7M1Q5, Q84VE0, Q84VE0 Oryza sativa (Rice); P45727 Persea americana (Avocado); Q9AXI5 Pharbitis nil (Violet; Japanese morning glory); P52777 Pinus taeda (Loblolly pine); Q01861, Q04593 Pisum sativum (Garden pea); P24481, P45728, P45729 Petroselinum crispum (Parsley; Petroselinum hortense); Q84LI2 PhalaenopsisxDoritaenopsis hybrid cultivar; P07218, P19142, P19143 Phaseolus vulgaris (Kidney bean; French bean); Q7XJC3, Q7XJC4 Pinus pinaster (Maritime pine); Q6UD65 Populus balsamifera subsp. trichocarpaxPopulus deltoides; P45731, Q43052, 024266 Populus kitakamiensis (Aspen); Q8H6V5, Q8H6V6 Populus tremuloides (Quaking aspen); P45730 Populus trichocarpa (Western balsam poplar); 064963 Prunus avium (Cherry); Q94ENO Rehmannia glutinosa; P11544 Rhodosporidium toruloides (Yeast) (Rhodotorula gracilis); P10248 Rhodotorula rubra (Yeast) (Rhodotorula mucilaginosa); Q9M568, Q9M567 Rubus idaeus (Raspberry); P35511, P26600 Solanum lycopersicum (Lycopersicon esculentum; Tomato); P31425, P31426 Solanum tuberosum (Potato); Q6SPE8 Stellaria longipes (Longstalk starwort); P45732 Stylosanthes humilis (Townsville stylo); P45734 Trifolium subterraneum (Subterranean clover); Q43210, Q43664 Triticum aestivum (Wheat); Q96V77 Ustilago maydis (Smut fungus); P45735 Vitis vinifera (Grape); and Q8VXG7 Zea mays (Maize).

In some aspects, PAL proteins expressed from polynucleotides provided herein are derived from plants. In some aspects, the plant PAL is an Arabidopsis PAL, a Solanum PAL, or a Nicotiana PAL. In other aspects, the plant PAL is an Arabidopsis PAL. In still other aspects, the plant PAL is an Arabidopsis thaliana PAL. In some aspects, the plant PAL is a Solanum PAL. In other aspects, the plant PAL is a Solanum lycopersicum PAL. In still other aspects, the plant PAL is a Nicotiana PAL. In further aspects, the plant PAL is a Nicotiana tabacum PAL.

In some aspects, polynucleotides provided herein encode a bacterial PAL protein having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to an Anabaena PAL protein. In other aspects, bacterial PAL encoded by polynucleotides provided herein has at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to an Anabaena variabilis PAL protein. In further aspects, bacterial PAL encoded by polynucleotides provided herein has at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to protein having a sequence of SEQ ID NO:17.

In some aspects, polynucleotides provided herein encode a plant PAL protein having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to an to an Arabidopsis PAL, a Solanum PAL, or a Nicotiana PAL protein. In other aspects, plant PAL encoded by polynucleotides provided herein has at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to an Arabidopsis thaliana PAL, a Solanum lycopersicum PAL, or a Nicotiana tabacum PAL protein. In further aspects, plant PAL encoded by polynucleotides provided herein has at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to protein having a sequence selected from SEQ ID NOs:18-20.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide and comparing these sequences to a second nucleotide or amino acid sequence. As used herein, the term “percent (%) sequence identity” or “percent (%) identity,” also including “homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul et al., J. Mol. Biol. 215:403-410 (1990); Karlin and Altschul, Proc. Natl. Acad. sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at ebi.ac.uk/Tools/psa/emboss needle/, optionally with default settings), the Smith-Waterman algorithm (see, e.g., the EMBOSS Water aligner available at ebi.ac.uk/Tools/psa/emboss water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group. 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

### Codon Optimization

In some embodiments, polynucleotides for expressing PAL proteins provided herein include codon-optimized sequences or regions. As used herein, the term “codon-optimized” means a polynucleotide, nucleic acid sequence, or coding sequence has been redesigned as compared to a wild-type or reference polynucleotide, nucleic acid sequence, or coding sequence by choosing different codons without altering the amino acid sequence of the encoded protein. Accordingly, codon-optimization generally refers to replacement of codons with synonymous codons to optimize expression of a protein while keeping the amino acid sequence of the translated protein the same. Codon optimization of a sequence can increase protein expression levels (Gustafsson et al., Codon bias and heterologous protein expression. 2004, Trends Biotechnol 22: 346-53) of the encoded proteins, for example, and provide other advantages. Variables such as codon usage preference as measured by codon adaptation index (CAI), for example, the presence or frequency of U and other nucleotides, mRNA secondary structures, cis-regulatory sequences, GC content, and other variables may correlate with protein expression levels (Villalobos et al., Gene Designer: a synthetic biology tool for constructing artificial DNA segments. 2006, BMC Bioinformatics 7:285).

Any method of codon optimization can be used to codon optimize polynucleotides and nucleic acid molecules provided herein, and any variable can be altered by codon optimization. Accordingly, any combination of codon optimization methods can be used. Exemplary methods include the high codon adaptation index (CAI) method, the Low U method, and others. The CAI method chooses a most frequently used synonymous codon for an entire protein coding sequence. As an example, the most frequently used codon for each amino acid can be deduced from 74,218 protein-coding genes from a human genome. The Low U method targets U-containing codons that can be replaced with a synonymous codon with fewer U moieties, generally without changing other codons. If there is more than one choice for replacement, the more frequently used codon can be selected. Any polynucleotide, nucleic acid sequence, or codon sequence provided herein can be codon-optimized. Codon optimization can be performed for increased or optimal expression in any species, including animals, plants, fungi, bacteria, protozoa, and others. Exemplary species include human, non-human primate, mouse, rabbit, and others.

In one aspect, polynucleotides for expressing bacterial PAL provided herein include a codon-optimized coding region encoding the bacterial PAL as compared to a wild-type or reference coding region encoding the bacterial PAL. As used herein, the terms “wild-type coding region” and “reference coding region” refer to a coding region that is not codon-optimized. The terms “wild-type coding region” and “reference coding region” may be used interchangeably, unless context clearly indicates otherwise. Accordingly, a wild-type coding region can encode a wild-type or a mutant PAL protein. Similarly, a reference coding region can encode a wild-type or a mutant PAL protein.

In some aspects, the bacterial PAL is a wild-type bacterial PAL. In other aspects, the bacterial PAL is a mutant bacterial PAL. In still other aspects, the coding region encoding the bacterial PAL includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9% identity to a sequence selected from SEQ ID NOs:1-4. In some aspects, the coding region encoding the bacterial PAL includes a sequence selected from SEQ ID NOs:1-4. Codon-optimized coding regions of polynucleotides provided herein and encoding bacterial PAL can be optimized according to mouse codon usage or human codon usage.

In one aspect, polynucleotides for expressing plant PAL provided herein include a codon-optimized coding region encoding the plant PAL as compared to a wild-type coding region encoding the plant PAL. In some aspects, the plant PAL is a wild-type plant PAL. In other aspects, the plant PAL is a mutant plant PAL. In still other aspects, the coding region encoding the plant PAL includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9% identity to a sequence selected from SEQ ID NOs:5-7. In some aspects, the coding region encoding the plant PAL includes a sequence selected from SEQ ID NOs:5-7. Codon-optimized coding regions of polynucleotides provided herein and encoding plant PAL can be optimized according to mouse codon usage or human codon usage. In one aspect, coding regions of polynucleotides provided herein and encoding plant PAL are codon-optimized according to the high codon adaptation index (CAI) method.

Mutant bacterial or plant PAL can include any mutation, including substitutions, deletions, insertions, and others, as well as combinations thereof. Coding regions for both wild-type and mutant PAL proteins can be codon-optimized. Where a coding region encoding a mutant PAL protein is codon-optimized, a reference sequence can be a PAL sequence that is wild-type except for one or more codons that include the mutation or alteration or a PAL sequence that is wild-type and includes one or more mutant codons that typically occur in the sequence. In some aspects, the mutant bacterial PAL includes a mutation at a position corresponding to C503, C565, or both C503 and C565 as compared to wild-type bacterial PAL. In some aspects, the mutant bacterial PAL includes a mutation at a position corresponding to C503, C565, or both C503 and C565 as compared to wild-type avPAL. In some aspects, mutant bacterial PAL includes a mutation corresponding to a mutation selected from C503S, C565S, or both C503S and C565S as compared to wild-type bacterial PAL. In other aspects, mutant Anabaena variabilis PAL (avPAL) comprises a mutation selected from C503S, C565S, or both C503S and C565S as compared to wild-type avPAL.

### Untranslated Regions and Other Elements

Polynucleotides encoding PAL proteins provided herein can further include untranslated regions (UTRs). In some aspects, polynucleotides provided herein include a 5′ UTR. In some aspects, the 5′ UTR is derived from an mRNA molecule known in the art to be relatively stable (e.g., histone, tubulin, globin, GAPDH, actin, or citric acid cycle enzymes) to increase the stability of the polynucleotide. In other embodiments, a 5′ UTR sequence may include a partial sequence of a CMV immediate-early 1 (IE1) gene. Examples of 5′ UTR sequences may be found in U.S. Pat. No. 9,149,506. In some aspects, the 5′ UTR includes a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK, AT1G58420, mouse beta globin, mouse albumin, and a tobacco etch virus, or fragments of any of the foregoing. In one aspect, the 5′ UTR is derived from a tobacco etch virus (TEV).

In some aspects, the 5′ UTR includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9% identity to a sequence of SEQ ID NO:8. In other aspects, polynucleotides provided herein include a 5′ UTR having a sequence of SEQ TD NO:8. In yet other aspects, the 5′ UTR of polynucleotides provided herein includes a fragment of a sequence of SEQ TD NO:8, such as a fragment of at least 10, 20, 30, 40, 50, 60, 70, 80, 90, 100, 110, 120, 125, 130, 135, 140, or 145, and any number or range in between, contiguous nucleotides of SEQ TD NO: 8. Additional exemplary 5′ UTR sequences of SEQ TD NOs:24-41 are shown in Table 1.

TABLE 1
Exemplary 5′ UTR Sequences

| SEQ | | |
|---|---|---|
| ID | | |
| NO. | SEQUENCE | SOURCE/NAME |
| 24 | UCAACACAACAUAUACAAAACAAACGAAUC | TEV |
| | UCAAGCAAUCAAGCAUUCUACUUCUAUUGC | |
| | AGCAAUUUAAAUCAUUUCUUUUAAAGCAAA | |
| | AGCAAUUUUCUGAAAAUUUUCACCAUUUAC | |
| | GAACGAUAG | |
| 25 | AUUAUUACAUCAAAACAAAAAGCCGCCA | AT1G58420 |
| 26 | AAUUAUUGGUUAAAGAAGUAUAUUAGUGC | HUMAN ALBUMIN |
| | UAAUUUCCCUCCGUUUGUCCUAGCUUUUCU | |
| | CUUCUGUCAACCCCACACGCCUUUGGCACA | |
| 27 | AACUUAAAAAAAAAAAUCAAA | SYNECHOCYSTIS sp. PCC6803 |
| | | POTASSIUM CHANNEL |
| | | (SynK) |
| 28 | CACAUUUGCUUCUGACAUAGUUGUGUUGAC | MOUSE BETA GLOBIN |
| | UCACAACCCCAGAAACAGACAUC | |
| 29 | ACAUUUGCUUCUGACACAACUGUGUUCACU | HUMAN BETA GLOBIN |
| | AGCAACCUCAAACAGACACC | |
| 30 | UGCACACAGAUCACCUUUCCUAUCAACCCC | MOUSE ALBUMIN |
| | ACUAGCCUCUGGCAAA | |
| 31 | AUAAAAAGACCAGCAGAUGCCCCACAGCAC | HUMAN HAPTOGLOBIN |
| | UGCUCUUCCAGAGGCAAGACCAACCAAG | |
| 32 | AGACAAGGUUCAUAUUUGUAUGGGUUACUU | HUMAN TRANSTHYRETIN |
| | AUUCUCUCUUUGUUGACUAAGUCAAUAAUC | |
| | AGAAUCAGCAGGUUUGCAGUCAGAUUGGCA | |
| | GGGAUAAGCAGCCUAGCUCAGGAGAAGUGA | |
| | GUAUAAAAGCCCCAGGCUGGGAGCAGCCAU | |
| | CACAGAAGUCCACUCAUUCUUGGCAGG | |
| 33 | AGAUAAAAAGCCAGCUCCAGCAGGCGCUGC | HUMAN COMPLEMENT C3 |
| | UCACUCCUCCCCAUCCUCUCCCUCUGUCCCU | |
| | CUGUCCCUCUGACCCUGCACUGUCCCAGCAC | |
| | C | |
| 34 | UAUAUCCGUGGUUUCCUGCUACCUCCAACC | HUMAN COMPLEMENT C5 |
| 35 | GGCACCACCACUGACCUGGGACAGUGAAUC | HUMAN ALPHA-1- |
| | GACA | ANTITRYPSIN |
| 36 | AUUCAUGAAAAUCCACUACUCCAGACAGAC | HUMAN ALPHA-1- |
| | GGCUUUGGAAUCCACCAGCUACAUCCAGCU | ANTICHYMOTRYPSIN |
| | CCCUGAGGCAGAGUUGAGA | |
| 37 | AAUAUUAGAGUCUCAACCCCCAAUAAAUAU | HUMAN INTERLEUKIN 6 |
| | AGGACUGGAGAUGUCUGAGGCUCAUUCUGC | |
| | CCUCGAGCCCACCGGGAACGAAAGAGAAGC | |
| | UCUAUCUCCCCUCCAGGAGCCCAGCU | |
| 38 | AGGAUGGGAACUAGGAGUGGCAGCAAUCCU | HUMAN FIBRINOGEN |
| | UUCUUUCAGCUGGAGUGCUCCUCAGGAGCC | ALPHA CHAIN |
| | AGCCCCACCCUUAGAAAAG | |
| 39 | AGGGGGAGCCCUAUAAUUGGACAAGUCUGG | HUMAN APOLIPOPROTEIN E |
| | GAUCCUUGAGUCCUACUCAGCCCCAGCGGA | |
| | GGUGAAGGACGUCCUUCCCCAGGAGCCGAC | |
| | UGGCCAAUCACAGGCAGGAAG | |
| 40 | AGACGGGUGGGGCGGGGCCCAACUGUCCCC | ALANINE |
| | AGCUCCUUCAGCCCUUUCUGUCCCUCCCAG | AMINOTRANSFERASE 1 |
| | UGAGGCCAGCUGCGGUGAAGAGGGUGCUCU | |
| | CUUGCCUGGAGUUCCCUCUGCUACGGCUGC | |
| | CCCCUCCCAGCCCUGGCCCACUAAGCCAGAC | |
| | CCAGCUGUCGCCAUUCCCACUUCUGGUCCU | |
| | GCCACCUCCUGAGCUGCCUUCCCGCCUGGUC | |
| | UGGGUAGAGUC | |
| 41 | UCUGCCCCACCCUGUCCUCUGGAACCUCUGC | HUMAN ANTITHROMBIN |
| | GAGAUUUAGAGGAAAGAACCAGUUUUCAGG | |
| | CGGAUUGCCUCAGAUCACACUAUCUCCACU | |
| | UGCCCAGCCCUGUGGAAGAUUAGCGGCC | |

In some aspects, polynucleotides provided herein include a Kozak sequence. As is understood in the art, a Kozak sequence is a short consensus sequence centered around the translational initiation site of eukaryotic mRNAs that allows for efficient initiation of translation of the mRNA. See, for example, Kozak, Marilyn (1988) Mol. and Cell Biol, 8:2737-2744; Kozak, Marilyn (1991) J. Biol. Chem, 266: 19867-19870; Kozak, Marilyn (1990) Proc Natl. Acad. Sci. USA, 87:8301-8305; and Kozak, Marilyn (1989) J. Cell Biol, 108:229-241. It ensures that a protein is correctly translated from the genetic message, mediating ribosome assembly and translation initiation. The ribosomal translation machinery recognizes the AUG initiation codon in the context of the Kozak sequence. A Kozak sequence may be inserted upstream of the coding sequence for the protein of interest, downstream of a 5′ UTR or inserted upstream of the coding sequence for the protein of interest and downstream of a 5′ UTR. A Kozak sequence can overlap with the 5′ UTR, the coding region, or both the 5′ UTR and the coding region. In some aspects, a polynucleotide described herein includes a Kozak sequence having the sequence GCCACC (SEQ ID NO: 21). In other aspects, a polynucleotide described herein includes a partial Kozak sequence having the sequence GCCA (SEQ ID NO: 22).

In some aspects, polynucleotides provided herein include a 3′ UTR. Examples of 3′ UTR sequences may be found in U.S. Pat. No. 9,149,506. In some aspects, the 3′ UTR includes a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, mouse MALAT-1, mouse beta globin, mouse albumin, and Xenopus beta globin, or fragments of any of the foregoing. In other aspects, the 3′ UTR is derived from Xenopus beta globin. In yet further aspects, the 3′ UTR is derived from Xenopus beta globin and contains one or more UNA monomers.

In some aspects, the 3′ UTR includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9% identity to a sequence of SEQ TD NO:9. In other aspects, polynucleotides provided herein include a 3′ UTR having a sequence of SEQ TD NO:9. In yet other aspects, the 3′ UTR of polynucleotides provided herein includes a fragment of a sequence of SEQ TD NO:9, such as a fragment of at least 10, 20, 30, 40, 50, 60, 70, 80, 90, 100, 110, 120, 125, 130, 135, 140, 145, 150, 155, 160, 165, 166, or 167, and any number or range in between, contiguous nucleotides of SEQ TD NO:9. Additional exemplary 3′ UTR sequences of SEQ TD NOs:42-56 are shown in Table 2.

TABLE 2
Exemplary 3′ UTR Sequences

| SEQ | | |
|---|---|---|
| ID | | |
| NO. | SEQUENCE | SOURCE/NAME |
| 42 | CUAGUGACUGACUAGGAUCUGGUUACCACUA | XBG |
| | AACCAGCCUCAAGAACACCCGAAUGGAGUCU | |
| | CUAAGCUACAUAAUACCAACUUACACUUACA | |
| | AAAUGUUGUCCCCCAAAAUGUAGCCAUUCGU | |
| | AUCUGCUCCUAAUAAAAAGAAAGUUUCUUCA | |
| | CAU | |
| 43 | UGCAAGGCUGGCCGGAAGCCCUUGCCUGAAA | HUMAN HAPTOGLOBIN |
| | GCAAGAUUUCAGCCUGGAAGAGGGCAAAGUG | |
| | GACGGGAGUGGACAGGAGUGGAUGCGAUAA | |
| | GAUGUGGUUUGAAGCUGAUGGGUGCCAGCCC | |
| | UGCAUUGCUGAGUCAAUCAAUAAAGAGCUUU | |
| | CUUUUGACCCAU | |
| 44 | ACGCCGAAGCCUGCAGCCAUGCGACCCCACG | HUMAN APOLIPOPROTEINE |
| | CCACCCCGUGCCUCCUGCCUCCGCGCAGCCUG | |
| | CAGCGGGAGACCCUGUCCCCGCCCCAGCCGU | |
| | CCUCCUGGGGUGGACCCUAGUUUAAUAAAGA | |
| | UUCACCAAGUUUCACGCA | |
| 45 | ACACAUCACAACCACAACCUUCUCAGGCUAC | MOUSE ALBUMIN |
| | CCUGAGAAAAAAAGACAUGAAGACUCAGGAC | |
| | UCAUCUUUUCUGUUGGUGUAAAAUCAACACC | |
| | CUAAGGAACACAAAUUUCUUUAAACAUUUGA | |
| | CUUCUUGUCUCUGUGCUGCAAUUAAUAAAAA | |
| | AUGGAAAGAAUCUAC | |
| 46 | GCUGGAGCCUCGGUAGCCGUUCCUCCUGCCC | HUMAN ALPHA GLOBIN |
| | GCUGGGCCUCCCAACGGGCCCUCCUCCCCUCC | |
| | UUGCACCGGCCCUUCCUGGUCUUUGAAUAAA | |
| | GUCUGAGUGGGCAGCA | |
| 47 | ACCCCCUUUCCUGCUCUUGCCUGUGAACAAU | MOUSE BETA GLOBIN |
| | GGUUAAUUGUUCCCAAGAGAGCAUCUGUCAG | |
| | UUGUUGGCAAAAUGAUAAAGACAUUUGAAA | |
| | AUCUGUCUUCUGACAAAUAAAAAGCAUUUAU | |
| | UUCACUGCAAUGAUGUUUU | |
| 48 | GCUCGCUUUCUUGCUGUCCAAUUUCUAUUAA | HUMAN BETA GLOBIN |
| | AGGUUCCUUUGUUCCCUAAGUCCAACUACUA | |
| | AACUGGGGGAUAUUAUGAAGGGCCUUGAGCA | |
| | UCUGGAUUCUGCCUAAUAAAAAACAUUUAUU | |
| | UUCAUUGCAA | |
| 49 | UGGCAUCCCUGUGACCCCUCCCCAGUGCCUC | HUMAN GROWTH FACTOR |
| | UCCUGGCCCUGGAAGUUGCCACUCCAGUGCC | |
| | CACCAGCCUUGUCCUAAUAAAAUUAAGUUGC | |
| | AUCAUUUUGUCUG | |
| 50 | AAUGUUCUUAUUCUUUGCACCUCUUCCUAUU | HUMAN ANTITHROMBIN |
| | UUUGGUUUGUGAACAGAAGUAAAAAUAAAU | |
| | ACAAACUACUUCCAUCUCA | |
| 51 | CCACACCCCCAUUCCCCCACUCCAGAUAAAG | HUMAN COMPLEMENT C3 |
| | CUUCAGUUAUAUCUCACGUGUCUGGAGUUCU | |
| | UUGCCAAGAGGGAGAGGCUGAAAUCCCCAGC | |
| | CGCCUCACCUGCAGCUCAGCUCCAUCCUACU | |
| | UGAAACCUCACCUGUUCCCACCGCAUUUUCU | |
| | CCUGGCGUUCGCCUGCUAGUGUG | |
| 52 | AACCUACCUGCCCUGCCCCCGUCCCCUCCCUU | HUMAN HEPCIDIN |
| | CCUUAUUUAUUCCUGCUGCCCCAGAACAUAG | |
| | GUCUUGGAAUAAAAUGGCUGGUUCUUUUGU | |
| | UUUCCAAA | |
| 53 | ACUAAGUUAAAUAUUUCUGCACAGUGUUCCC | HUMAN FIBRINOGEN |
| | AUGGCCCCUUGCAUUUCCUUCUUAACUCUCU | ALPHA CHAIN |
| | GUUACACGUCAUUGAAACUACACUUUUUUGG | |
| | UCUGUUUUUGUGCUAGACUGUAAGUUCCUUG | |
| | GGGGCAGGGCCUUUGUCUGUCUCAUCUCUGU | |
| | AUUCCCAAAUGCCUAACAGUACAGAGCCAUG | |
| | ACUCAAUAAAUACAUGUUAAAUGGAUGAAU | |
| | GAAUUCCUCUGAAACUCU | |
| 54 | GCACCCCAGCUGGGGCCAGGCUGGGUCGCCC | ALANINE |
| | UGGACUGUGUGCUCAGGAGCCCUGGGAGGCU | AMINOTRANSFERASE 1 |
| | CUGGAGCCCACUGUACUUGCUCUUGAUGCCU | |
| | GGCGGGGUGGGGUGGGGGGGGUGCUGGGCCC | |
| | CUGCCUCUCUGCAGGUCCCUAAUAAAGCUGU | |
| | GUGGCAGUCUGACUCC | |
| 55 | GAUUCGUCAGUAGGGUUGUAAAGGUUUUUC | MOUSE MALAT-1 |
| | UUUUCCUGAGAAAACAACCUUUUGUUUUCUC | |
| | AGGUUUUGCUUUUUGGCCUUUCCCUAGCUUU | |
| | AAAAAAAAAAAAGCAAAA | |
| 56 | GGACGC | ALANINE |
| | CUCAGGCACC GGAGCCAGAC CCUCCCAAGA | AMINOTRANSFERASE |
| | CCACCCAGGC CUUCCUCAAG GACUCUGCCU | |
| | CAGACCUCAG ACAGGCCACC AACGCUGUUC | |
| | AUCUUCAUUU CCCCAAGGAG ACUUCUUUCU | |
| | UUGUGCCUUG AUGUUUGAGA GUUCUUCGAG | |
| | CAAACAGUGG UUUUGCAAUG UCUCACAGGC | |
| | CCUGUUUUUG UUUUUGUUUU UGUUUUGUUU | |
| | UGUUUUGUUC UUUUUUUAAA UGCAACCAAA | |
| | GUAGAGUCAA CCUGCUCGGC AGAUGUACUU | |
| | GGAUUCUCUG AAUCGCUAUU CUGUUUGGAG | |
| | AGUUCCUUUG GGUCUUAAGC AGCCAGAGUA | |
| | CAUGGAAAUG AGAUUAUGUC AGAUCUGGAG | |
| | AAACAAGCAG GUGUUGGGAA AUAUGUGACU | |
| | UGACAUGAUA AGGGCUGGGA AUCCAGAAAU | |
| | CAAUAGUGAG AUCCAUGAAA UCAAACCCUG | |
| | ACCAGUGUGA AAAUGUAGCC UUUUGGACAG | |
| | UAAGCCUGCA AGUCUAGUGA GAACUCAGAG | |
| | AAAGCUGACC AUUCUGGUCU GAAGAUAGGC | |
| | AGCGCAUCAC AGGCAAGAAU AUCGAAGUCA | |
| | GUAGUAGGAC AGGGGUCACA UCAGAUACCA | |
| | GCUCAAAUUG CACUAGCUAU CUAGAACAGU | |
| | UUUCUCCAGG UUUGCCUGAG CCUUGAUGCA | |
| | UACCAUCGCC CUCUGCUGGU CGCAGCAGAG | |
| | AUAAGCAAGG GCUGAAAAUG GAGGCAAUCC | |
| | UUUCCCAAGG CCCUGAAAGU UGUUUUUCAU | |
| | GGUUUCAAAC UGAAUUUGGC UCAUUUGUAA | |
| | CUAACUGAUC ACGGUGCCUG GUUACACUGG | |
| | CUGCCAAGAA GGAGCGCAUG CAAUCUGAUU | |
| | CAGUGCUCUC UUCACAUCAG UUUCCUGCCU | |
| | CCCUCCCUCA UCUGCGGACA GCAUCCUAUC | |
| | UCAUCAGGCU UCCCUGUGUG UCACAAAGUA | |
| | GCAGCCACCA AGCAAAUAUA UUCCUUGAAU | |
| | UAGCACACCU GGGUGGGCCA UGUGCGCACC | |
| | AAGGAAACAG GUGCUAUAGG GAGCGCCAGG | |
| | CCAGGCUUGU CUCUUAACUG UCUCGUUCUU | |
| | CAGUGAGAGU GGGAAAGCUG UCCGGAGCUC | |
| | CCGCGCAGGA GCCUGGGUAC CCACGCAGCG | |
| | AGUCAAGGGA GUUUUCGGAG CCAGAGAGAG | |
| | AAAGAUGUGA AGGCUGUGGA GUAAGGCUGA | |
| | AACCAGCCUC CUGCCCUAUA GUCCCACACU | |
| | GCAGGGGGUG CGACUUUAAA ACAGAACUUC | |
| | AAGUUGUUAA CACUCACAAG CAUUGCAUUA | |
| | CUGUGAAGGA AGUAGCCGCA UCCAUAACAG | |
| | GAUGUGAUGG UCUACAGCUU UUCCUUUAAA | |
| | AGCUGAAAAG GUACCAUGUG UGCUCGCUAG | |
| | GCAUAUAAUC CAGAUAUGCU CCAGAGUUCU | |
| | GAGAUUCUUC CAUGAAAGGU UAACUAGAAG | |
| | CUAGAAUAUU UUUUUAUAUU UUUGUAACAA | |
| | UUGGCUUUUU UCAUGGGGGG AGGGGAGUAG | |
| | AGGGUUAGUA UUUAUAGUCC UAACAAGUCC | |
| | AAAAAUUUUU AUAAGUGUCU UCAGAUUAUA | |
| | AAUAACCCUC CAAAUUUUGC AAUGUUUACA | |
| | UGUUUUUUUU UUAAGAUGAC AAAUAUGCUU | |
| | GAUUUGCUUU UUAAAUAAAA GUUUAGCUGU | |
| | UCUAAGAGAU UAACUUCAAG UAGGAUGGCU | |
| | GGUUAUGAUA GUUUGGAUUU UCUACAGGUU | |
| | CUGUUGCCAU GCCUUUUGGG UUUCAGCAUC | |
| | ACUCGAGUCG CAGCAUGUGG GUGGGGCUGU | |
| | GGAAACCUGG CCAGGCUGGA CCUGGUCAGC | |
| | CACACCUCAG AGACAUUGUU UCCAUUUGGA | |
| | UGUGAGCAGG CGCAGGCCUG CAUGCUCUUU | |
| | CCUACUUAGC AUCAUCAGUU CUUCCGCCUC | |
| | CUUAGCAUGG UUCUUUGUAA CAGCCAUGCU | |
| | GGGAAGCUCU GAACAAUAAA AUACUUCCAG | |
| | AGUGGU | |

In some aspects, polynucleotides provided herein include a sequence of a triple stop codon immediately downstream of the coding sequence or coding region. A triple stop codon can enhance translation efficiency. In some aspects, polynucleotides provided herein include a sequence of AUAAGUGAA (SEQ ID NO: 23).

### Tail Region

In some aspects, polynucleotides provided herein further include a tail region. A tail region can protect a polynucleotide, such as an mRNA, from exonuclease degradation. In some aspects, the tail region is a poly(A) tail or poly(A) sequence.

Poly(A) tails can be added using a variety of methods known in the art, e.g., using poly(A) polymerase to add tails to synthetic or in vitro transcribed RNA. Other methods include the use of a transcription vector to encode poly(A) tails or the use of a ligase (e.g., via splint ligation using a T4 RNA ligase and/or T4 DNA ligase), wherein poly(A) may be ligated to the 3′ end of a sense RNA. In some embodiments, a combination of any of the above methods is utilized.

In some aspects, polynucleotides provided herein include a poly(A) tail or poly(A) sequence. The length of the poly(A) tail or poly(A) sequence can be at least about 5, 10, 15, 20, 25, 30, 35, 40, 45, 50, 60, 70, 80, 90, 100, 200, or 300 nucleotides, and any number or range in between. In some aspects, the poly(A) tail or poly(A) sequence is at least about 5 to 300 nucleotides, e.g., at least about 5 to 25 nucleotides, at least about 5 to 50 nucleotides, at least about 5 to 100 nucleotides, at least about 5 to 150 nucleotides, at least about 5 to 200 nucleotides, at least about 5 to 250 nucleotides, or at least about 5 to 300 nucleotides. In one aspect, the poly(A) tail or poly(A) sequence is at least about 80 nucleotides. In another aspect, the poly(A) tail or poly(A) sequence is at least about 90 nucleotides. In one aspect, the poly(A) tail or poly(A) sequence is at least about 100 nucleotides.

In some aspects, polynucleotides provided herein include a poly(C) tail or poly(C) sequence. The length of the poly(C) tail or poly(C) sequence can be at least about 5, 10, 15, 20, 25, 30, 35, 40, 45, 50, 60, 70, 80, 90, 100, 200, or 300 nucleotides, and any number or range in between. In some aspects, the poly(C) tail or poly(C) sequence is at least about 5 to 300 nucleotides, e.g., at least about 5 to 25 nucleotides, at least about 5 to 50 nucleotides, at least about 5 to 100 nucleotides, at least about 5 to 150 nucleotides, at least about 5 to 200 nucleotides, at least about 5 to 250 nucleotides, or at least about 5 to 300 nucleotides. In one aspect, the poly(C) tail or poly(C) sequence is at least about 100 nucleotides.

### RNA Molecules

Polynucleotides provided herein can be DNA molecules or RNA molecules. It will be appreciated that T present in DNA is substituted with U in RNA, and vice versa. In some aspects, polynucleotides provided herein are RNA molecules. An RNA molecule provided herein can be generated by in vitro transcription (IVT) of DNA molecules. In one aspect, RNA molecules provided herein are mRNA molecules. In another aspect, RNA molecules provided herein are self-replicating RNA molecules. In yet another aspect, RNA molecules provided herein further include a 5′ cap. Any 5′ cap can be included in RNA molecules provided herein, including 5′ caps having a Cap 1 structure, a Cap 1 (m⁶A) structure, a Cap 2 structure, a Cap 0 structure, or any combination thereof. In one aspect, RNA molecules provided herein include a 5′ cap having Cap 1 structure. In yet another aspect, RNA molecules provided herein are mRNA or self-replicating RNA molecules including a 5′ cap having a Cap 1 structure. In a further aspect, RNA molecules provided herein include a cap having a Cap 1 structure, wherein a m⁷G is linked via a 5′-5′ triphosphate to the 5′ end of the 5′ UTR. In yet a further aspect, RNA molecules provided herein include a cap having a Cap 1 structure, wherein a m⁷G is linked via a 5′-5′ triphosphate to the 5′ end of the 5′ UTR including a sequence of SEQ ID NO:8. Any method of capping can be used, including, but not limited to using a Vaccinia Capping enzyme (New England Biolabs, Ipswich, Mass.) and co-transcriptional capping or capping at or shortly after initiation of in vitro transcription (IVT), by for example, including a capping agent as part of an in vitro transcription (IVT) reaction. (Nuc. Acids Symp. (2009) 53:129).

In some aspects, RNA molecules provided herein include chemically modified nucleotides that include modified nucleosides. In one aspect, RNA molecules that include chemically modified nucleotides are mRNA molecules. Any modified nucleotide can be included in RNA molecules provided herein. Chemically modified nucleotides of RNA molecules provided herein can include modified nucleosides with modified bases, for example. In some aspects, modified nucleotides of RNA molecules provided herein include chemically modified nucleosides selected from 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine, 6-O-methylguanosine, and any combination thereof. In one aspect, the chemically modified nucleosides are N¹-methylpseudouridines. In another aspect, the chemically modified nucleosides are 5-methoxyuridines.

Any percentage or number of nucleotides of RNA molecules provided herein can be chemically modified. In one aspect, chemically modified nucleotides include 1-100% of the nucleotides that can be chemically modified. In another aspect, chemically modified nucleotides include 50-100% of the nucleotides that can be chemically modified. As an example, where the chemically modified nucleotides include modified uridine, 1 to 100% or 50 to 100% of uridines can be chemically modified. As another example, where the chemically modified nucleotide includes adenosines, 1 to 100% or 50 to 100% of adenosines can be chemically modified. As yet another example, where the chemically modified nucleotides include modified uridines and modified adenosines, 1 to 100% or 50 to 100% of uridines and adenosines can be chemically modified, with any proportion of uridines and any proportion of adenosines being modified. Accordingly, where more than one type of nucleoside or nucleoside base of nucleotides included in RNA molecules provided herein is modified, such as two, three, or four types of nucleosides or nucleoside bases, any proportion of nucleosides or nucleoside bases can be modified for a total percentage of 1 to 100% or 50 to 100% chemical modification.

In some aspects, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, and any number or range in between, of nucleotides in RNA molecules provided herein are chemically modified. Chemical modifications can include N¹-methylpseudouridines, 5-methoxyuridines, or a combination of N¹-methylpseudouridines and 5-methoxyuridines.

RNA molecules provided herein can have a length of about 50 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 5,500 nucleotides, about 6,000 nucleotides, about 6,500 nucleotides, about 7,000 nucleotides, about 7,500 nucleotides, about 8,000 nucleotides, about 8,500 nucleotides, about 9,000 nucleotides, about 9,500 nucleotides, about 10,000 nucleotides, about 11,000 nucleotides, about 12,000 nucleotides, about 13,000 nucleotides, about 14,000 nucleotides, about 15,000 nucleotides, about 16,000 nucleotides, about 17,000 nucleotides, about 18,000 nucleotides, about 19,000 nucleotides, about 20,000 nucleotides, and any number or range in between.

### DNA Molecules

In one aspect, provided herein are DNA molecules encoding the polynucleotides provided herein. In another aspect, DNA molecules encoding polynucleotides provided herein include a promoter. As used herein, the term “promoter” refers to a regulatory sequence that initiates transcription. A promoter can be operably linked to an open reading frame or a coding sequence. A promoter can also be operably linked to a gene that includes a 5′ UTR, an open reading frame or a coding sequence, a 3′ UTR, a sequence encoding a poly(A) or poly(C) tail, a triple stop codon, or any combination thereof.

Generally, promoters included in DNA molecules provided herein include promoters for in vitro transcription (IVT). Any suitable promoter for in vitro transcription can be included in DNA molecules provided herein, such as a T7 promoter, a T3 promoter, an SP6 promoter, and others. In one aspect, DNA molecules provided herein include a T7 promoter. In another aspect, the promoter is located 5′ of a 5′ UTR included in DNA molecules provided herein. In yet another aspect, the promoter is a T7 promoter located 5′ of a 5′ UTR included in DNA molecules provided herein. In yet another aspect, the promoter overlaps with the 5′ UTR. A promoter and a 5′ UTR can overlap by about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, or more nucleotides.

In some aspects, DNA molecules provided herein include a promoter for in vivo transcription. Generally, the promoter for in vivo transcription is an RNA polymerase II (RNA pol II) promoter. Any RNA pol II promoter can be included in DNA molecules provided herein, including constitutive promoters, inducible promoters, and tissue-specific promoters. Exemplary constitutive promoters include a cytomegalovirus (CMV) promoter, an EF1α promoter, an SV40 promoter, a PGK1 promoter, a Ubc promoter, a human beta actin promoter, a CAG promoter, and others. Any tissue-specific promoter can be included in DNA molecules provided herein. In one aspect, the RNA pol II promoter is a liver-specific promoter, muscle-specific promoter, skin-specific promoter, subcutaneous tissue-specific promoter, spleen-specific promoter, lymph node-specific promoter, or a promoter with any other tissue specificity. DNA molecules provided herein can also include an enhancer. Any enhancer that increases transcription can be included in DNA molecules provided herein.

### Compositions and Pharmaceutical Compositions

Provided herein, in some embodiments, are compositions and pharmaceutical compositions that include a polynucleotide provided herein and a pharmaceutically acceptable carrier. In one aspect, the pharmaceutically acceptable carrier includes a lipid formulation. The lipid formulation can be a transfection reagent, a lipoplex, a liposome, a lipid nanoparticle, a polymer-based carrier, an exosome, a lamellar body, a micelle, or an emulsion. In one aspect, the lipid formulation is a liposome. In another aspect, the lipid formulation is a liposome selected from a cationic liposome, a nanoliposome, a proteoliposome, a unilamellar liposome, a multilamellar liposome, a ceramide-containing nanoliposome, and a multivesicular liposome. In yet another aspect, the lipid formulation is a lipid nanoparticle. The lipid nanoparticle can encapsulate polynucleotides provided herein.

Any lipid can be included in lipid formulations of compositions and pharmaceutical compositions provided herein. In one aspect, lipid formulations of compositions and pharmaceutical compositions provided herein include a cationic lipid. In another aspect, the cationic lipid included in lipid formulations is an ionizable cationic lipid. Any ionizable cationic lipid can be included in compositions and pharmaceutical compositions that include polynucleotides provided herein. Exemplary ionizable cationic lipids include the following:

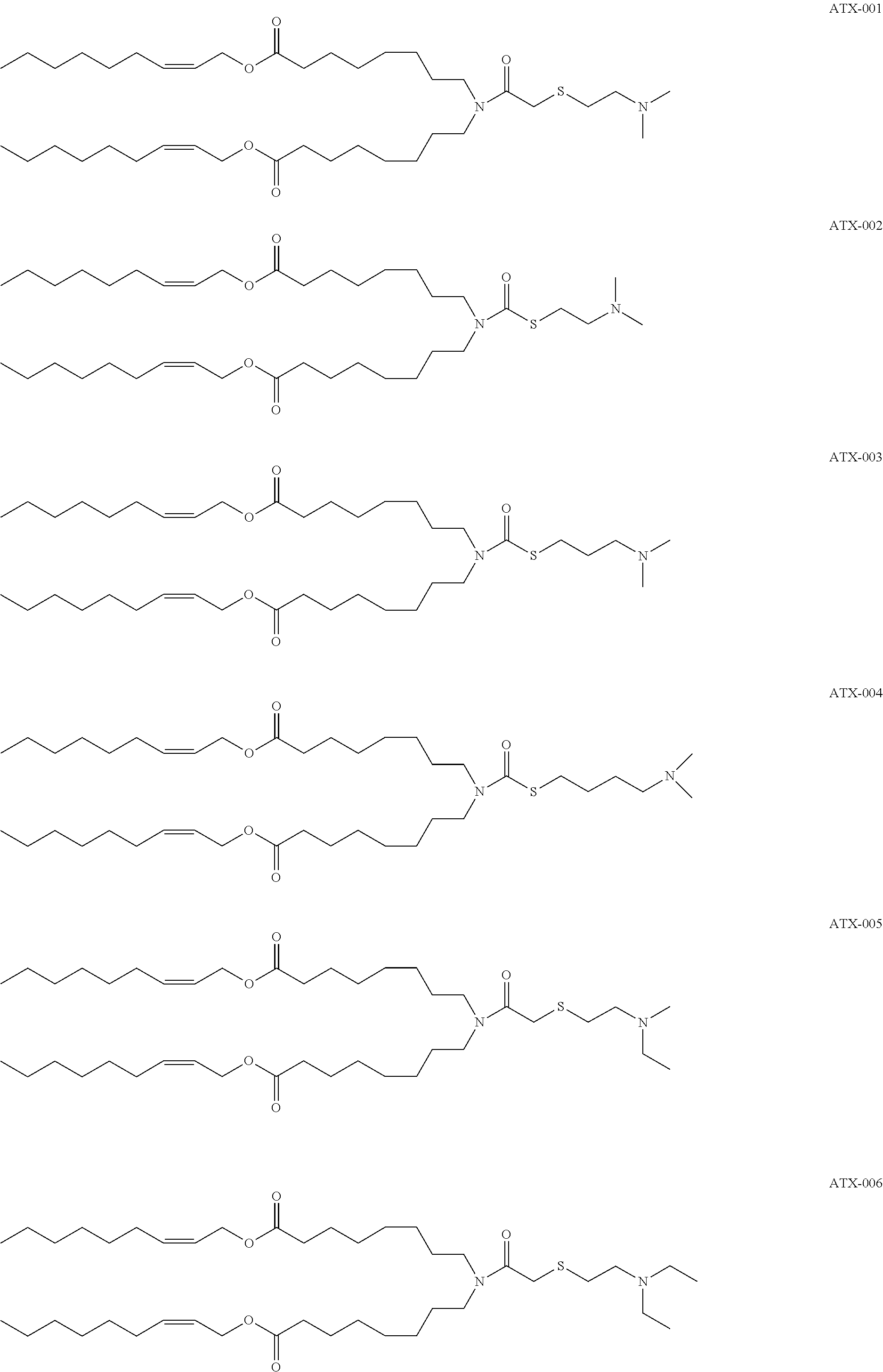

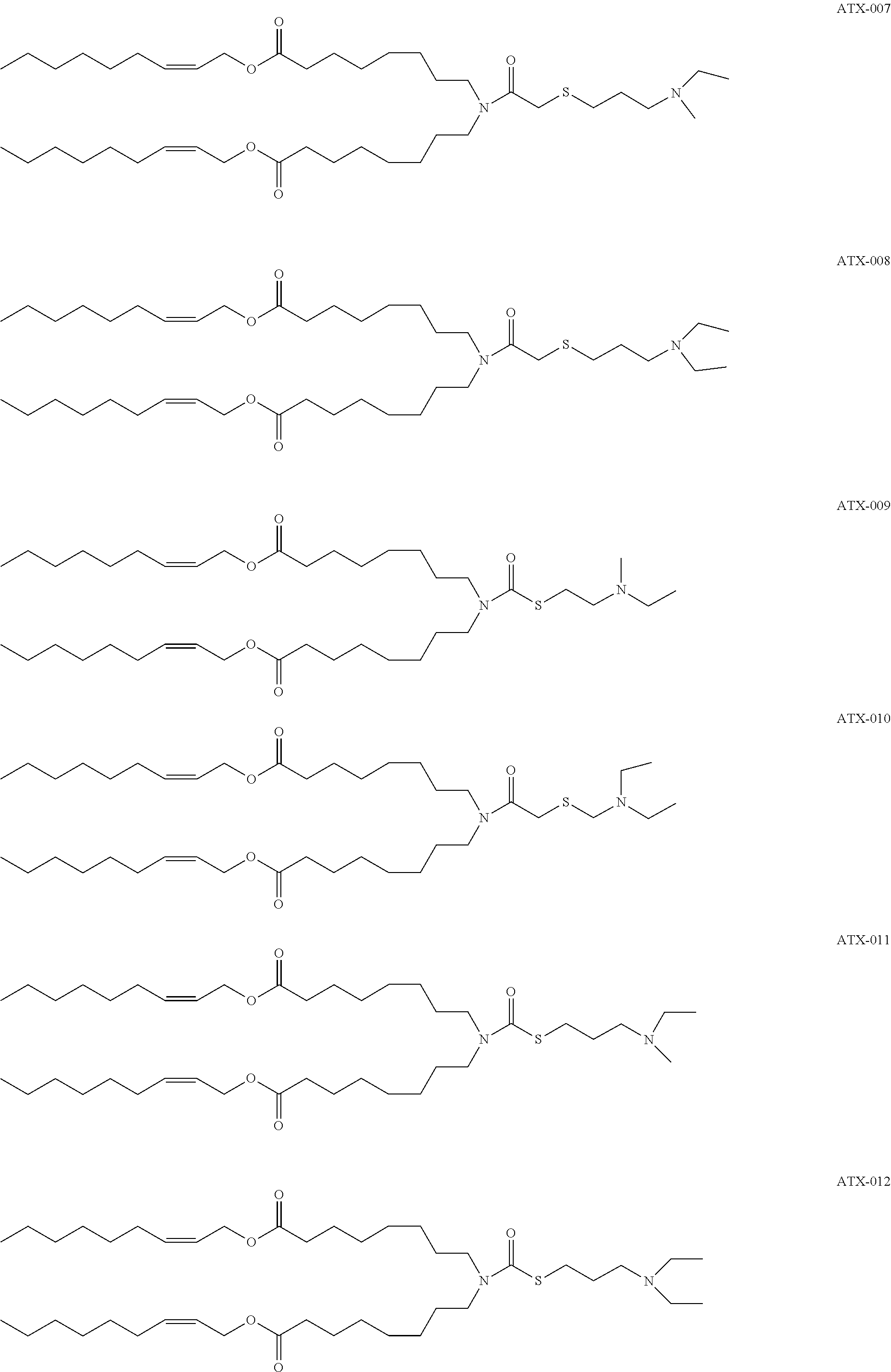

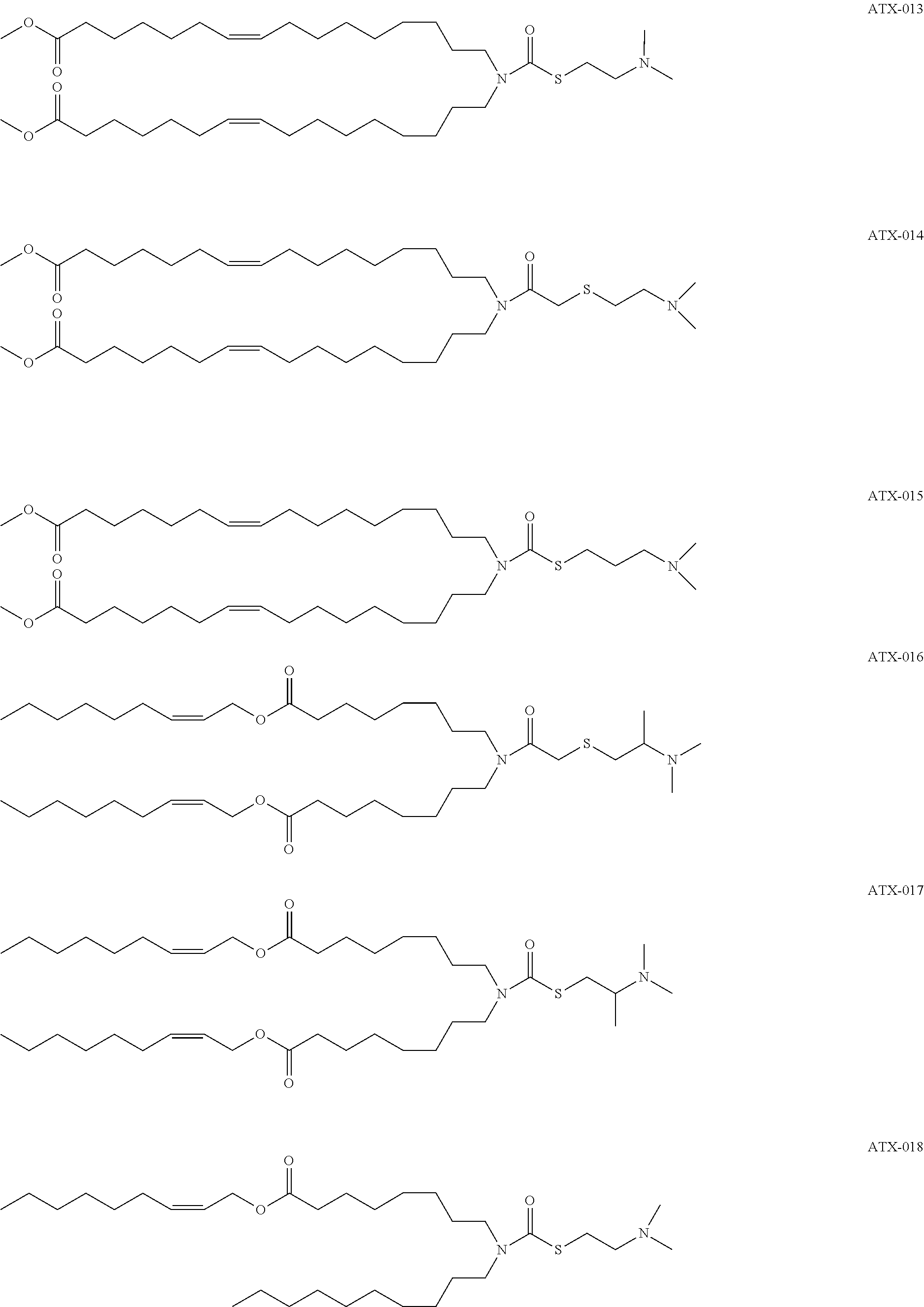

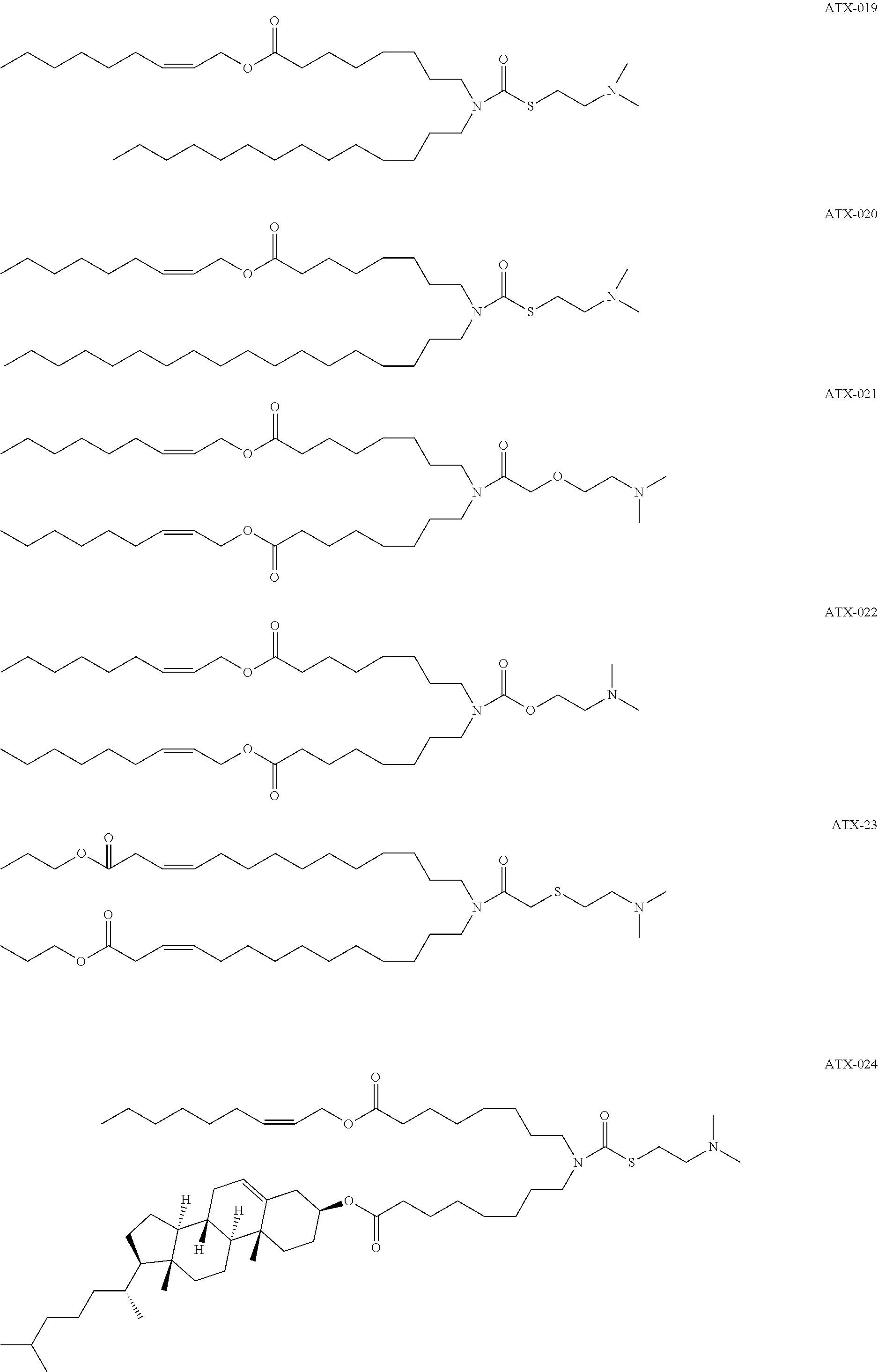

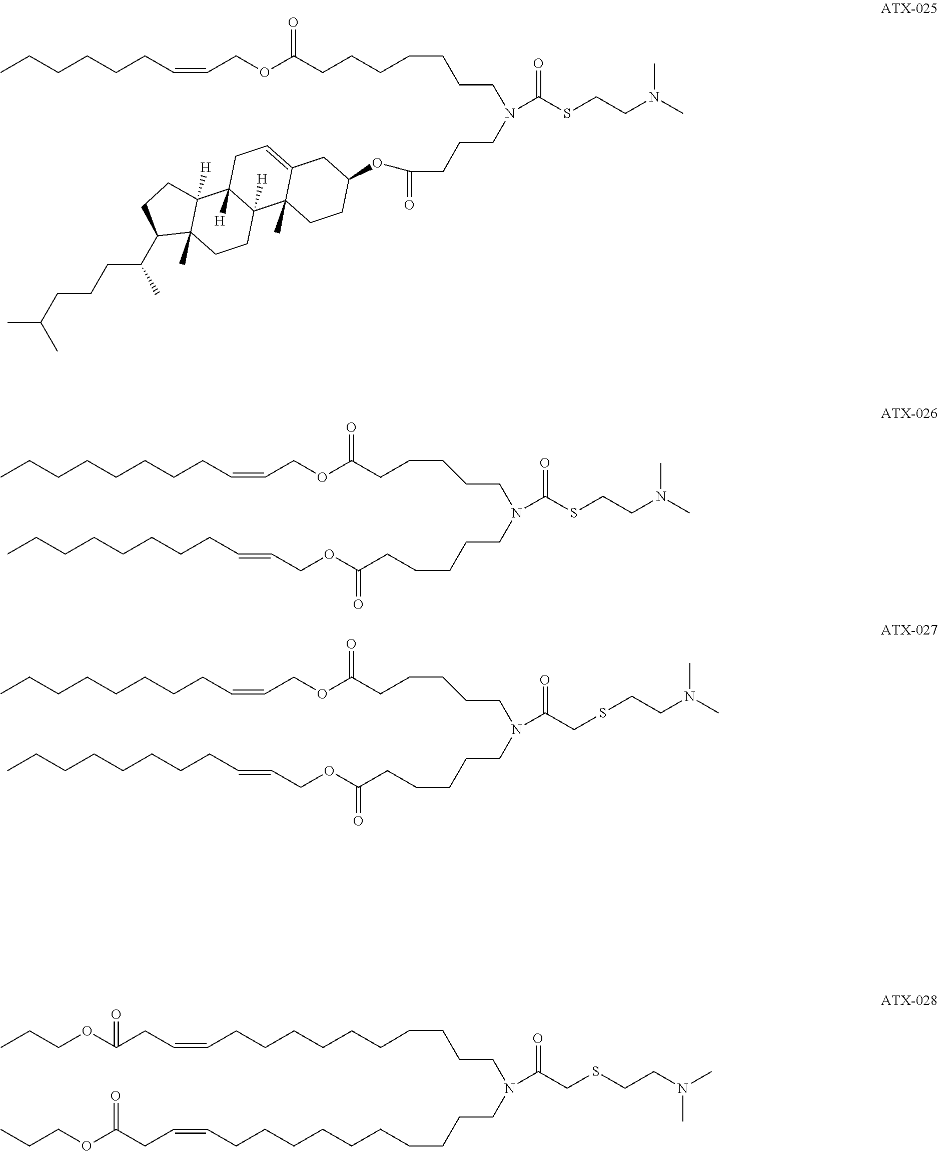

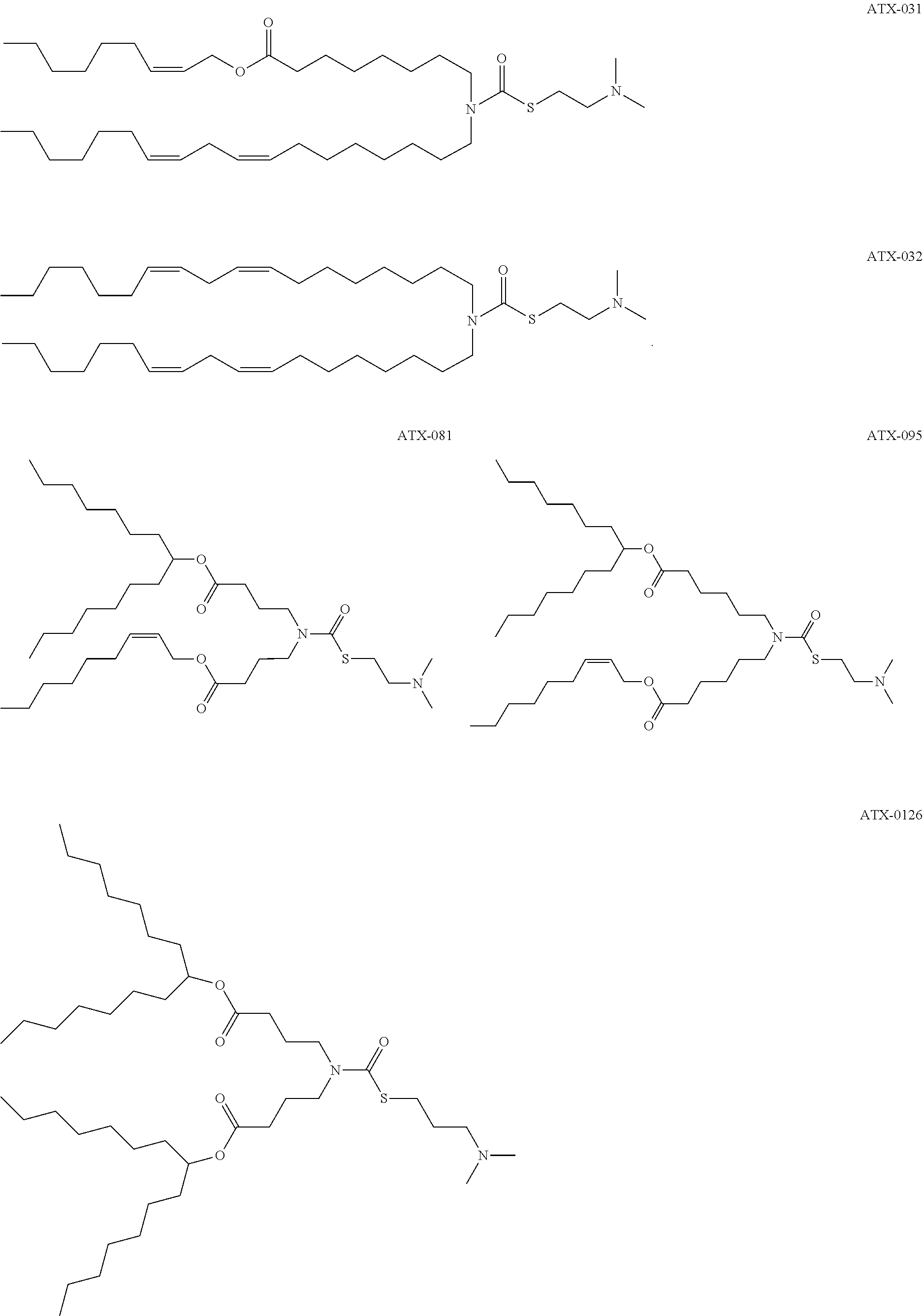

### Lipid Formulations/LNPs

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), release at the cytoplasm (for RNA), and so on. Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment; however, some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed herein below.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is generally formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (imparted by the cationic lipid) result in rapid plasma clearance, hemolytic and other toxicities, as well as immune system activation. In some aspects, nucleic acid molecules provided herein and lipids or lipid formulations provided herein form a lipid nanoparticle (LNP).

In other aspects, nucleic acid molecules provided herein are incorporated into a lipid formulation (i.e., a lipid-based delivery vehicle).

In the context of the present disclosure, a lipid-based delivery vehicle typically serves to transport a desired RNA to a target cell or tissue. The lipid-based delivery vehicle can be any suitable lipid-based delivery vehicle known in the art. In some aspects, the lipid-based delivery vehicle is a liposome, a cationic liposome, or a lipid nanoparticle containing an RNA of the disclosure. In some aspects, the lipid-based delivery vehicle comprises a nanoparticle or a bilayer of lipid molecules and an RNA of the disclosure. In some aspects, the lipid bilayer further comprises a neutral lipid or a polymer. In some aspects, the lipid formulation comprises a liquid medium. In some aspects, the formulation further encapsulates a nucleic acid. In some aspects, the lipid formulation further comprises a nucleic acid and a neutral lipid or a polymer. In some aspects, the lipid formulation encapsulates the nucleic acid.

Provided herein are lipid formulations that include one or more RNA molecules encapsulated within the lipid formulation. In some aspects, the lipid formulation comprises liposomes. In some aspects, the lipid formulation comprises cationic liposomes. In some aspects, the lipid formulation comprises lipid nanoparticles.

In some aspects, the RNA is fully encapsulated within the lipid portion of the lipid formulation such that the RNA in the lipid formulation is resistant in aqueous solution to nuclease degradation. In other aspects, the lipid formulations described herein are substantially non-toxic to animals such as humans and other mammals.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 45:1, or from about 10:1 to about 40:1, or from about 15:1 to about 40:1, or from about 20:1 to about 40:1; or from about 25:1 to about 45:1; or from about 30:1 to about 45:1; or from about 32:1 to about 42:1; or from about 34:1 to about 42:1. In some aspects, the total lipid:RNA ratio (mass/mass ratio) is from about 30:1 to about 45:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, generally are resistant in aqueous solution to degradation with a nuclease.

In some embodiments, the lipid nanoparticle has a size of less than about 500 nm, less than about 400 nm, less than about 300 nm, less than about 200 nm, less than about 100 nm, or less than about 50 nm. In specific embodiments, the lipid nanoparticle has a size of about 55 nm to about 90 nm.

In some aspects, the lipid formulations comprise an RNA, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol. In one aspect, the cationic lipid is an ionizable cationic lipid.

In the nucleic acid-lipid formulations, the RNA may be fully encapsulated within the lipid portion of the formulation, thereby protecting the nucleic acid from nuclease degradation. In some aspects, a lipid formulation comprising an RNA is fully encapsulated within the lipid portion of the lipid formulation, thereby protecting the nucleic acid from nuclease degradation. In certain aspects, the RNA in the lipid formulation is not substantially degraded after exposure of the particle to a nuclease at 37° C. for at least 20, 30, 45, or 60 minutes. In certain other aspects, the RNA in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In some aspects, the RNA is complexed with the lipid portion of the formulation. One of the benefits of the formulations of the present disclosure is that the nucleic acid-lipid compositions are substantially non-toxic to animals such as humans and other mammals.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I₀−I)/I₀, where I and I₀ refer to the fluorescence intensities before and after the addition of detergent.

In some aspects, the present disclosure provides a nucleic acid-lipid composition comprising a plurality of nucleic acid-liposomes, nucleic acid-cationic liposomes, or nucleic acid-lipid nanoparticles. In some aspects, the nucleic acid-lipid composition comprises a plurality of RNA-liposomes. In some aspects, the nucleic acid-lipid composition comprises a plurality of RNA-cationic liposomes. In some aspects, the nucleic acid-lipid composition comprises a plurality of RNA-lipid nanoparticles.

In some aspects, the lipid formulations comprise RNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the RNA encapsulated therein. The amount may be any value or subvalue within the recited ranges, including endpoints. The RNA included in any RNA-lipid composition or RNA-lipid formulation provided herein can be an mRNA or a self-replicating RNA.

Depending on the intended use of the lipid formulation, the proportions of the components can be varied, and the delivery efficiency of a particular formulation can be measured using assays known in the art.

In some aspects, nucleic acid molecules provided herein are lipid formulated. The lipid formulation is preferably selected from, but not limited to, liposomes, cationic liposomes, and lipid nanoparticles. In one aspect, a lipid formulation is a cationic liposome or a lipid nanoparticle (LNP) comprising:
(a) an RNA of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In another aspect, the cationic lipid is an ionizable cationic lipid. Any ionizable cationic lipid can be included in lipid formulations, including exemplary cationic lipids provided herein.

### Cationic Lipids

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P-(N-(N′,N′-dimethylaminoethane)-carbamoyl) cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. Nos. 8,158,601; and Love et al., PNAS, 107(5), 1864-69, 2010, the contents of which are herein incorporated by reference.

The RNA-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. As a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In some aspects, the cholesterol derivative is a polar analogue such as cholesteryl-(4′-hydroxy)-butyl ether.

In some aspects, the helper lipid present in the lipid formulation comprises or consists of a mixture of one or more phospholipids and cholesterol or a derivative thereof. In other aspects, the neutral lipid present in the lipid formulation comprises or consists of one or more phospholipids, e.g., a cholesterol-free lipid formulation. In yet other aspects, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C₄ to C₂₂ may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. It will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I.

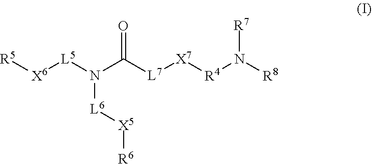

or a pharmaceutically acceptable salt or solvate thereof, wherein R5 and R6 are each independently selected from the group consisting of a linear or branched C₁-C₃₁ alkyl, C₂-C₃₁ alkenyl or C₂-C₃₁ alkynyl and cholesteryl; L5 and L6 are each independently selected from the group consisting of a linear C₁-C₂₀ alkyl and C₂-C₂₀ alkenyl; X5 is —C(O)O—, whereby —C(O)O—R6 is formed or —OC(O)— whereby —OC(O)—R6 is formed; X6 is —C(O)O— whereby —C(O)O—R5 is formed or —OC(O)— whereby —OC(O)—R5 is formed; X7 is S or O; L7 is absent or lower alkyl; R4 is a linear or branched C₁-C₆ alkyl; and R7 and R8 are each independently selected from the group consisting of a hydrogen and a linear or branched C1-C6 alkyl.

In some embodiments, X7 is S.

In some embodiments, X5 is —C(O)O—, whereby —C(O)O—R6 is formed and X6 is —C(O)O— whereby —C(O)O—R5 is formed.

In some embodiments, R7 and R8 are each independently selected from the group consisting of methyl, ethyl and isopropyl.

In some embodiments, L5 and L6 are each independently a C₁-C₁₀ alkyl. In some embodiments, L5 is C₁-C₃ alkyl, and L6 is C₁-C₅ alkyl. In some embodiments, L6 is C₁-C₂ alkyl. In some embodiments, L5 and L6 are each a linear C₇ alkyl. In some embodiments, L5 and L6 are each a linear C₉ alkyl.

In some embodiments, R5 and R6 are each independently an alkenyl. In some embodiments, R6 is alkenyl. In some embodiments, R6 is C₂-C₉ alkenyl. In some embodiments, the alkenyl comprises a single double bond. In some embodiments, R5 and R6 are each alkyl. In some embodiments, R5 is a branched alkyl. In some embodiments, R5 and R6 are each independently selected from the group consisting of a C₉ alkyl, C₉ alkenyl and C₉ alkynyl. In some embodiments, R5 and R6 are each independently selected from the group consisting of a C₁₁ alkyl, C₁₁ alkenyl and C₁₁ alkynyl. In some embodiments, R5 and R6 are each independently selected from the group consisting of a C₇ alkyl, C₇ alkenyl and C₇ alkynyl. In some embodiments, R5 is —CH((CH₂)pCH₃)₂ or —CH((CH₂)pCH₃)((CH₂)p-1CH₃), wherein p is 4-8. In some embodiments, p is 5 and L5 is a C₁-C₃ alkyl. In some embodiments, p is 6 and L5 is a C₃ alkyl. In some embodiments, p is 7. In some embodiments, p is 8 and L5 is a C₁-C₃ alkyl. In some embodiments, R5 consists of —CH((CH₂)pCH₃)((CH₂)p-1CH₃), wherein p is 7 or 8.

In some embodiments, R4 is ethylene or propylene. In some embodiments, R4 is n-propylene or isobutylene.

In some embodiments, L7 is absent, R4 is ethylene, X7 is S and R7 and R8 are each methyl. In some embodiments, L7 is absent, R4 is n-propylene, X7 is S and R7 and R8 are each methyl. In some embodiments, L7 is absent, R4 is ethylene, X7 is S and R7 and R8 are each ethyl.

In some embodiments, X7 is S, X5 is —C(O)O—, whereby —C(O)O—R6 is formed, X6 is —C(O)O— whereby —C(O)O—R5 is formed, L5 and L6 are each independently a linear C₃-C₇ alkyl, L7 is absent, R5 is —CH((CH₂)pCH₃)₂, and R6 is C₇-C₁₂ alkenyl. In some further embodiments, p is 6 and R6 is C₉ alkenyl.

In embodiments, any one or more lipids recited herein may be expressly excluded.

In some aspects, the helper lipid comprises from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The lipid portion, or the cholesterol or cholesterol derivative in the lipid formulation may comprise up to about 40 mol %, about 45 mol %, about 50 mol %, about 55 mol %, or about 60 mol % of the total lipid present in the lipid formulation. In some aspects, the cholesterol or cholesterol derivative comprises about 15 mol % to about 45 mol %, about 20 mol % to about 40 mol %, about 25 mol % to about 35 mol %, or about 28 mol % to about 35 mol %; or about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, or about 37 mol % of the total lipid present in the lipid formulation.

In specific embodiments, the lipid portion of the lipid formulation is about 35 mol % to about 42 mol % cholesterol.

In some aspects, the phospholipid component in the mixture may comprise from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The percentage of helper lipid present in the lipid formulation is a target amount, and the actual amount of helper lipid present in the formulation may vary, for example, by ±5 mol %.

A lipid formulation that includes a cationic lipid compound or ionizable cationic lipid compound may be on a molar basis about 30-70% cationic lipid compound, about 25-40% cholesterol, about 2-15% helper lipid, and about 0.5-5% of a polyethylene glycol (PEG) lipid, wherein the percent is of the total lipid present in the formulation. In some aspects, the composition is about 40-65% cationic lipid compound, about 25-35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2-25% cholesterol, 10-35% cholesterol-PEG, and 5% cholesterol-amine; or 2-30% cationic lipid, 2-30% helper lipid, 1-15% cholesterol, 2-35% cholesterol-PEG, and 1-20% cholesterol-amine; or up to 90% cationic lipid and 2-10% helper lipids, or even 100% cationic lipid.

### Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front Pharmacol. 2015 Dec. 1; 6:286).

In some aspects, the lipid conjugate is a PEG-lipid. The inclusion of polyethylene glycol (PEG) in a lipid formulation as a coating or surface ligand, a technique referred to as PEGylation, helps to protect nanoparticles from the immune system and their escape from RES uptake (Nanomedicine (Lond). 2011 June; 6(4):715-28). PEGylation has been used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (Journal of Nanomaterials. 2011; 2011:12). PEGylation leads to a significant increase in the circulation half-life of lipid formulations (Annu. Rev. Biomed. Eng. 2011 Aug. 15; 13 ( ):507-30; J. Control Release. 2010 Aug. 3; 145(3):178-81).

Examples of PEG-lipids include, but are not limited to, PEG coupled to dialkyloxypropyls (PEG-DAA), PEG coupled to diacylglycerol (PEG-DAG), methoxypolyethyleneglycol (PEG-DMG or PEG2000-DMG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH2).

The PEG moiety of the PEG-lipid conjugates described herein may comprise an average molecular weight ranging from about 550 daltons to about 10,000 daltons. In certain aspects, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In some aspects, the PEG moiety has an average molecular weight of about 2,000 daltons or about 750 daltons. The average molecular weight may be any value or subvalue within the recited ranges, including endpoints.

In certain aspects, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one aspect, the linker moiety is a non-ester-containing linker moiety. Exemplary non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, as well as combinations thereof (such as a linker containing both a carbamate linker moiety and an amido linker moiety). In one aspect, a carbamate linker is used to couple the PEG to the lipid.

In some aspects, an ester-containing linker moiety is used to couple the PEG to the lipid. Exemplary ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some aspects, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₅) conjugate. In these embodiments, the PEG preferably has an average molecular weight of about 750 or about 2,000 daltons. In particular embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some aspects, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

In some embodiments, the lipid formulation for any of the compositions described herein comprises a lipoplex, a liposome, a lipid nanoparticle, a polymer-based particle, an exosome, a lamellar body, a micelle, or an emulsion.

### Mechanism of Action for Cellular Uptake of Lipid Formulations

In some aspects, lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately enters and moves through the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For RNA payloads, the cell's own internal translation processes will then translate the RNA into the encoded protein. The encoded protein can further undergo posttranslational processing, including transportation to a targeted organelle or location within the cell or excretion from the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual assymetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique, a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified as continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross-junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly, lipid and plasmid are solubilized in a detergent solution of appropriate ionic strength, and after removing the detergent by dialysis, a stabilized lipid formulation is formed. Unencapsulated nucleic acid is then removed by ion-exchange chromatography and empty vesicles are removed by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

### Excipients

Pharmaceutical compositions provided herein can be formulated using one or more excipients to: (1) increase stability; (2) increase cell transfection; (3) permit a sustained or delayed release (e.g., from a depot formulation of the polynucleotide, primary construct, or RNA); (4) alter the biodistribution (e.g., target the polynucleotide, primary construct, or RNA to specific tissues or cell types); (5) increase the translation of encoded protein in vivo; and/or (6) alter the release profile of encoded protein in vivo.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally include a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or RNA (e.g., for transplantation into a subject), hyaluronidase, nanoparticle mimics and combinations thereof.

Accordingly, the pharmaceutical compositions described herein can include one or more excipients, each in an amount that together increases the stability of the nucleic acid in the lipid formulation, increases cell transfection by the nucleic acid, increases the expression of the encoded protein, and/or alters the release profile of encoded proteins. Further, the RNA of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The pharmaceutical compositions of this disclosure may further contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the RNA-lipid formulation may be administered in a time-release formulation, for example in a composition which includes a slow-release polymer. The active agent can be prepared with carriers that will protect against rapid release, for example a controlled release vehicle such as a polymer, microencapsulated delivery system, or a bioadhesive gel. Prolonged delivery of the RNA, in various compositions of the disclosure can be brought about by including in the composition agents that delay absorption, for example, aluminum monostearate hydrogels and gelatin.

In one aspect, lipid formulations of compositions and pharmaceutical compositions provided herein include a cationic lipid or an ionizable cationic lipid, and further include at least one other lipid selected from the group consisting of anionic lipids, zwitterionic lipids, neutral lipids, steroids, polymer conjugated lipids, phospholipids, glycolipids, and combinations thereof.

### Methods of Treatment

Provided herein, in some embodiments, are methods for ameliorating, preventing, delaying onset, or treating a disease or condition associated with phenylketonuria, phenylalanine hydroxylase (PAH) deficiency, decreased metabolism of phenylalanine, or increased levels of phenylalanine in a subject. Methods provided herein can include administering to the subject any polynucleotide or composition or pharmaceutical composition provided herein.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “in need of treatment” as used herein refers to a judgment made by a caregiver (e.g., physician, nurse, nurse practitioner, or individual in the case of humans; veterinarian, veterinary technician, or other individual in the case of animals, including non-human mammals) that a subject requires or will benefit from treatment. This judgment is made based on a variety of factors that are in the realm of a caregiver's expertise, but that include the knowledge that the subject is ill, or will be ill, as the result of a condition that is treatable by the compositions of the invention.

As used herein, the term “effective amount” or “therapeutically effective amount” or “therapeutically effective dose” refers to that amount of a nucleic acid molecule, composition, or pharmaceutical composition described herein that is sufficient to effect the intended application, including but not limited to condition or disease treatment, as defined herein. The therapeutically effective amount may vary depending upon the intended application (e.g., treatment of a disease or condition, application in vivo), or the subject or patient and disease or condition being treated, e.g., the weight and age of the subject, the species, the severity of the disease or condition, the manner of administration and the like, which can readily be determined by one of ordinary skill in the art. The term also applies to a dose that will induce a particular response in a target cell. The specific dose will vary depending on the particular polynucleotide or nucleic acid molecule, composition, or pharmaceutical composition chosen, the dosing regimen to be followed, whether it is administered in combination, and other parameters.

Exemplary doses of polynucleotides or nucleic acid molecules that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between. In one aspect, the polynucleotides or nucleic acid molecules are RNA molecules. In another aspect, the polynucleotides or nucleic acid molecules are DNA molecules. Polynucleotides or nucleic acid molecules can have a unit dosage comprising about 0.01 μg to about 1,000 μg or more nucleic acid in a single dose.

In some aspects, compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid. In other aspects, pharmaceutical compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid formulation.

In one aspect, compositions provided herein can have a unit dosage that includes about 0.01 μg to about 1,000 μg or more nucleic acid and lipid in a single dose. In another aspect, pharmaceutical compositions provided herein can have a unit dosage that includes about 0.01 μg to about 1,000 μg or more nucleic acid and lipid formulation in a single dose. A unit dosage can correspond to the unit dosage of nucleic acid molecules, compositions, or pharmaceutical compositions provided herein and that can be administered to a subject. In one aspect, compositions of the instant disclosure have a unit dosage that includes about 0.01 μg to about 1,000 μg or more nucleic acid and lipid formulation in a single dose. In another aspect, compositions of the instant disclosure have a unit dosage that includes about 0.01 μg to about 500 μg nucleic acid and lipid formulation in a single dose. In yet another aspect, compositions of the instant disclosure have a unit dosage that includes about 0.01 μg to about 100 μg nucleic acid and lipid formulation in a single dose.

In one aspect, administering a polynucleotide, composition, or pharmaceutical composition provided herein increases expression of the bacterial or plant PAL protein or a fragment thereof in the liver, serum, plasma, kidney, heart, muscle, brain, cerebrospinal fluid, lymph nodes, or any combination thereof, as compared with administering a control polynucleotide or a control composition or vehicle. In another aspect, administering a polynucleotide, composition, or pharmaceutical composition provided herein decreases blood phenylalanine levels, increases blood trans-cinnamic acid (tCA) levels, increases blood hippurate (HA) levels, or any combination thereof, as compared with administering a control polynucleotide or a control composition or vehicle. In yet another aspect, administering a polynucleotide, composition, or pharmaceutical composition provided herein includes a therapeutically effective dose of from 0.01 mg/kg to 10 mg/kg.

As used herein, the terms “reduce,” “decrease,” “reduction,” “minimal,” “low,” or “lower” refer to decreases below basal or reference levels, e.g., as compared to a control. The terms “increase,” high,” “higher,” “maximal,” “elevate,” or “elevation” refer to increases above basal or reference levels, e.g., as compared to a control. Increases, elevations, decreases, or reductions can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to a control or standard level. Each of the values or ranges recited herein may include any value or subrange therebetween, including endpoints.

Any route of administration can be included in methods provided herein. In some aspects, administration of polynucleotides or nucleic acid molecules, compositions, and pharmaceutical compositions is intravenous, subcutaneous, intradermal, transdermal, intranasal, oral, sublingual, intraperitoneal, intramuscular, topical, or by a pulmonary route. In some embodiments, administration may occur by implantation of a slow-release device, e.g., a mini-osmotic pump, to a subject. Administration may be by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, and the like. In embodiments, the administering does not include administration of any active agent other than the recited active agent. In embodiments, administration of compositions described herein is by intranasal administration such as inhalation or nebulization. In embodiments, administration may be pulmonary delivery via nasal or oral administration (e.g. by aerosolization or nebulization). In other aspects, administration of polynucleotides or nucleic acid molecules, compositions, and pharmaceutical compositions is intravenous.

By “co-administer” or co-administration” it is meant that a composition described herein is administered at the same time, just prior to, or just after the administration of one or more additional therapies. The compounds provided herein can be administered alone or can be co-administered to the patient. Co-administration is meant to include simultaneous or sequential administration of the compounds individually or in combination (more than one compound). Thus, the preparations can also be combined, when desired, with other active substances.

### Materials and Methods for Examples 1-2

### In Vitro Transcription (IVT) for Synthesis

Mouse codon-optimized PAL sequences (Anabaena variabilis or Trichormus variabilis PAL (Q3M5Z3), Arabipdosis thaliana at PAL (P35510), Solanum lycopersicum slPAL (P35511), Nicotiana tabacum NtPAL (P25872) were cloned into plasmids containing a T7 promoter, 5′ UTR, in frame with a Myc tag coding sequence, 3′UTR and poly(A) tail. The cloned portions of all plasmid constructs were verified by DNA sequencing. Plasmids were linearized immediately after the poly(A) stretch and used as templates for in vitro transcription reactions with T7 RNA polymerase. The RNA was synthesized with 100% substitution of UTP with N1-methyl-pseudo-UTP or 5-Methoxy-UTP, as indicated. The reaction for RNA was performed as previously described (10) with modifications to allow highly efficient co-transcriptional incorporation of a Cap1 analogue (Anti-Reverse Cap Analog (ARCA) 3′O-Me-m⁷G(5′)ppp(5′)G, New England Biolabs, Cat #S1411L) and to achieve high quality mRNA molecule transcription. RNA was then purified through a silica column (Macherey Nagel) and quantified by UV absorbance. For the in vivo experiments, the RNA quality and integrity were verified by 0.8-1.2% non-denaturing agarose gel electrophoresis as well as on a Fragment Analyzer (Advanced Analytical). The purified RNAs were stored in RNase-free water at −80° C. until further use.

Cell Culture PAL mRNA Transfections for PAL Expression and Activity

Transfections were performed using Lipofectamine® MessengerMAX™ transfection reagent (Thermo Fisher Scientific) according to the manufacturer's instructions. Mouse Hepa1-6 cells (ATCC) were plated in 96 well plates the day before transfection. DMEM medium containing 10% FBS was replaced immediately before beginning the transfection experiment. Medium was collected at desired time points post-transfection and 100 μl fresh medium was added into each well. Medium was kept at −80° C. until Phe or tCA analysis was performed. After deproteinizing media samples with a 10 Kda MWCO spin filter, Phe was quantified using a Phenylalanine Assay Kit (Sigma). Cells were also collected at the same time points for protein analysis by western blot.

### tCA Quantification by Thin Laver Chromatography (TLC)

Conditioned medium was collected from Hepa1-6 cell cultures and proteins precipitated with ethyl acetate and 0.1% formic acid. After centrifugation, the upper layer was extracted and dried out. The precipitate was resuspended in methanol and spotted on TLC glass membranes for trans-cinnamic acid (tCA) detection, in parallel to a control of pure tCA. The mobile phase was chloroform:methanol:formic acid (85:15:1).

### PKU Animal Model

Pah^{enu2}/J homozygous mice were obtained from The Jackson Laboratory. Prior to dosing, animals were group-housed (up to 5/cage); from the time of dosing, animals were single-housed. Mice were housed in microisolator caging in ventilated racks. Environmental controls for the animal room generally targeted a temperature range of 23±3° C. and a relative humidity range of 50±20% with a 12-hour/12-hour light/dark cycle. Throughout the study, mice were offered Teklad Global 18% protein rodent diet (Envigo RMX, Inc.) and water ad libitum. All animals were aged 2-4 months on the day of dosing.

Mice were housed in a pathogen-free environment and all mouse studies were approved by the Explora Biolabs Institutional Animal Care and Use Committee (IACUC) and performed according to Animal Care and Use Protocols.

### Blood Collection

Prior to dosing (“0 hours”), and at the indicated time points post-dose blood was collected from each animal by retro-orbital bleeding. For each time point, blood was collected into K₂EDTA-containing tubes and processed to plasma by centrifugation. The resulting plasma was stored at −80° C. until transferred for evaluation.

### Plasma Phe and Hippuric Acid (HA) Concentration Measurements

All in vivo plasma samples were assessed for Phe and HA concentrations by LC-MS/MS at JadeBio (La Jolla, CA). In brief, for each sample, plasma was diluted in water and protein was precipitated by combining with methanol containing each of the internal standards (¹³C-6-Phe and ¹³C-6-HA). The precipitate was pelleted, and the supernatants were transferred to a fresh microtiter plate and subjected to LC-MS/MS for chromatographic separation.

### PAL Protein Quantification in Mouse Liver and Mouse Cells by Western Blot

Proteins were extracted from liver tissue using Precellys Lysing Kit tubes and RIPA buffer including a cocktail of protease inhibitors. After lysing the tissue using Precellys 24, samples were briefly sonicated and centrifuged and the supernatant was kept for standard western blot analysis. Protein extraction from Hepa1-6 cells was performed by sonication for 4 cycles of 30 seconds on ice with a 1-minute interval in RIPA buffer containing a cocktail of protease inhibitor (Complete, Roche). Before loading, samples were normalized, loading the same amount of protein for each sample in the same western blot.

Immunoblotting was performed on PVDF Membranes using a LI-COR Quantitative Western Blot system. PAL was detected using Rabbit Anti-Myc polyclonal antibody (AbCam Cat. No. ab9106) and Donkey Anti-Rabbit IRDye 680 RD (LICOR) as the primary and secondary antibodies. Beta-actin (β-Actin; a housekeeping protein used as a loading control) was detected using Mouse Anti-Actin antibody (AbCam Cat. No. ab6276) as the primary antibody and Donkey Anti-Mouse IgG −800CW (LICOR) as the secondary antibody. After secondary antibody incubation and washing, membranes were scanned and analyzed using an Oddisey system to obtain western images and quantify band intensity.

### Lipid Nanoparticle Formulations

Lipid nanoparticles (LNPs) were prepared essentially as described (10). For the encapsulation of mRNA, mRNA was dissolved in 5 mM citric acid buffer, pH 3.5, whereas lipids were dissolved in ethanol. The molar percentage ratio for the constituent lipids was 50% ionizable amino lipid (Arcturus Therapeutics) or MC3, 7% DSPC (1,2-distearoyl-sn-glycero-3-phosphocholine) (Avanti Polar Lipids), 40% cholesterol (Avanti Polar Lipids), and 3% DMG-PEG (1,2-Dimyristoyl-sn-glycerol, methoxypolyethylene glycol, PEG chain molecular weight: 2000) (NOF America Corporation). The lipid and mRNA solutions were then combined in a Nanossemblr microfluidic device (Precision NanoSystems) at a flow ratio of 1:3 (ethanol:aqueous phase). The total combined flow rate was 12 mL/min. Lipid nanoparticles thus formed were purified by dialysis against phosphate buffer overnight using Spectra/Por Flot-a-lyzer ready to use dialysis device (Spectrum Labs) followed by concentration using Amicon Ultra-15 centrifugal filters (Merck Millipore). Particle size was determined by dynamic light scattering (ZEN3600, Malvern Instruments). Encapsulation efficiency was calculated by determining unencapsulated RNA content by measuring the fluorescence upon the addition of RiboGreen (Molecular Probes) to the LNP slurry (Fi) and comparing this value to the total RNA content that obtained upon lysis of the LNPs by 1% Triton X-100 (Ft), where percentage of encapsulation=(Ft−Fi)/Ft×100.

### Statistical Analysis

Where appropriate, values are expressed as means±SEM. Groups were compared by nonpaired two-tailed heteroscedastic t-tests using GraphPad Prism software. A p value <0.05 was considered significant.

### Example 1

This example describes in vitro and in vivo expression of PAL protein.

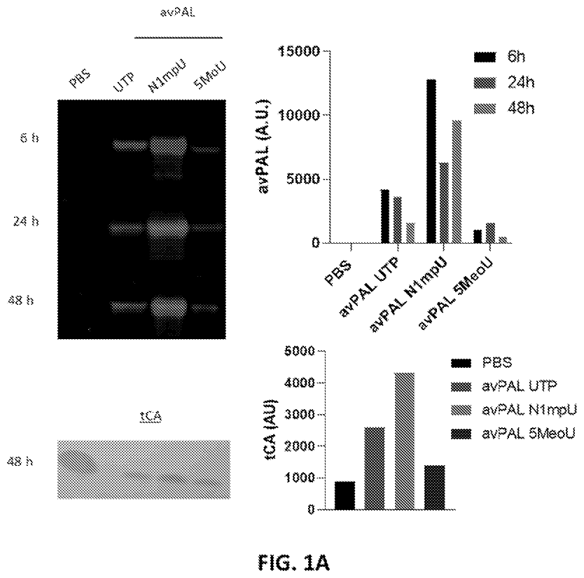

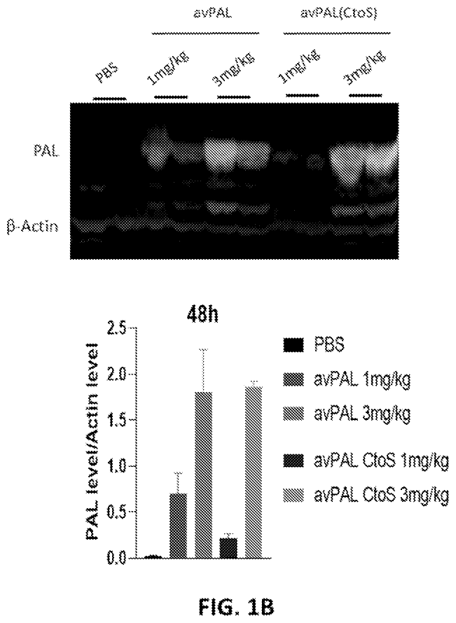

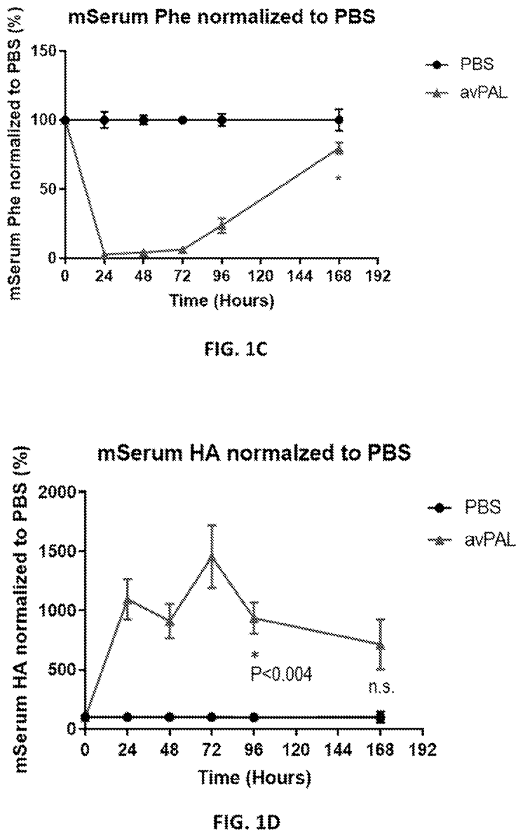

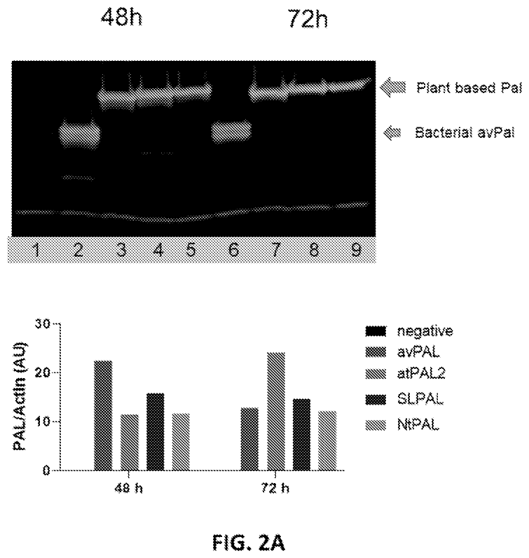

In vitro studies showed positive expression for all three avPAL protein variants prepared by in vitro transcription using UTP, N1 mpU (N1), or 5-methoxyuridine (5 MoU), as shown in the western blot and accompanying thin layer chromatography (TLC) (FIG. 1A.) PAL protein expression was evident in the cells after 6 hours and was still present at the 48-hour time point. TLC results also showed the presence of the Phe metabolite tCA, indicating that the PAL protein was biologically active and metabolizing Phe to tCA. As can be seen in the bar charts (FIG. 1A), avPAL N1 in lane 3 showed the highest level of protein expression, and the greatest amount of tCA. Therefore, avPAL N1 was selected for knockout mouse model studies.

Wild-type and mutant versions of avPAL N1 were delivered to a PKU mouse model using the LNP delivery system to determine both the dose response and in vivo stability of the mRNA, together with the duration of expression and the activity of the resultant PAL protein. FIG. 1B shows the expression level of the PAL proteins in vivo at 1 mg/kg and 3 mg/kg dosing levels. Both versions of the protein showed dose-dependent expression, with similar expression levels at the higher (3 mg/kg) dose, but lower expression levels of a mutant PAL protein (C503S/C565S; avPAL(CtoS)) as compared to wild-type avPAL at the 1 mg/kg dose (FIG. 1B).

To determine the activity of the PAL protein in vivo, the level of serum Phe was measured at 24-hour time points out to 96 hours, and again at 168 hours. The results shown in FIG. 1C confirmed that the wild-type PAL protein was biologically active and metabolizing Phe at 96 hours and beyond. Serum levels of the Phe metabolite hippurate (HA) (FIG. 1D), measured at the same time points, revealed a concordant increase in the level of HA out to the 96-hour time point.

These results show that active PAL protein was expressed in vitro and in vivo.

### Example 2

This example describes comparisons of bacterial and plant-derived PAL.

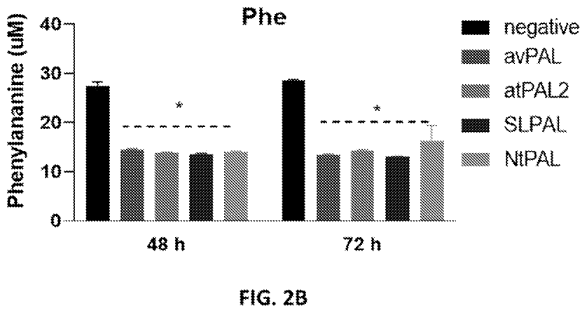

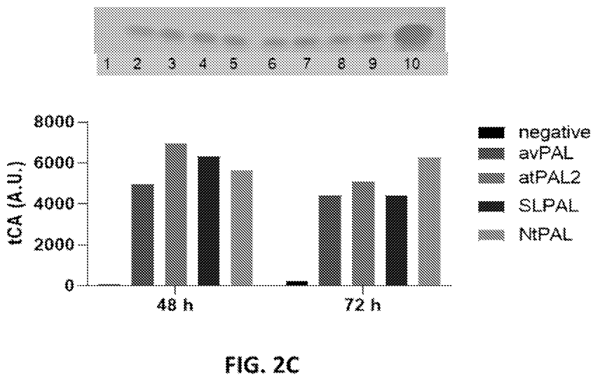

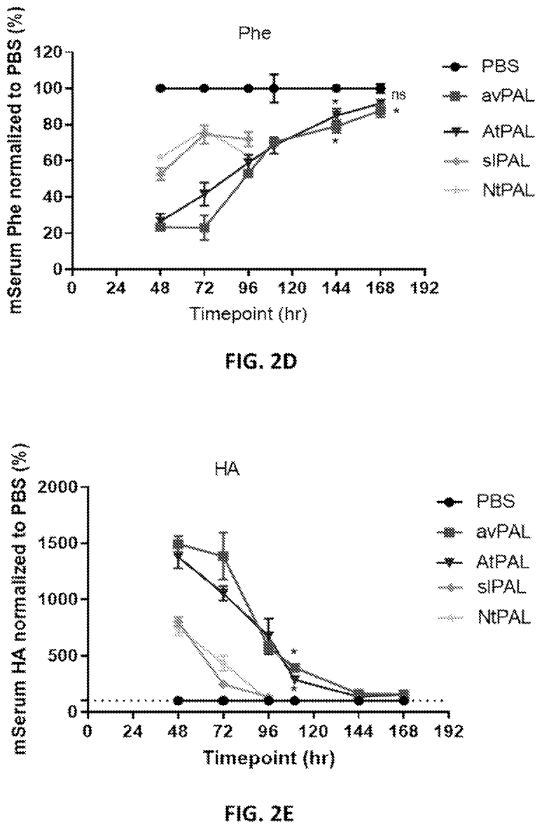

The results of further experiments to compare the transfection, expression, and biological activity of three plant-derived PAL proteins versus bacterial avPAL are shown in FIGS. 2A-2E. Western blot results at the 48-hour and 72-hour time points (FIG. 2A) showed similar levels of protein expression in cell culture for all four PAL proteins. Phe levels (FIG. 2B) were also reduced to a comparable level by all four PAL variants, and the presence of tCA was established by TLC (FIG. 2C).

The three plant PAL variants and avPAL were transfected into PKU mice using LNP technology and serum levels of Phe and HA were again measured at time points from 48 hours to 168 hours post-transfection. FIG. 2D shows serum Phe levels for all four PAL variants. Phe levels were reduced in the presence of plant PAL variants and avPAL as compared to PBS, demonstrating the stability and biological activity of the PAL proteins out to the 120-hour time point and beyond. FIG. 2E shows the serum levels of HA in the same mice and measured at the same time points. Greater HA levels were seen in the presence of plant PAL variants and avPAL as compared to PBS, with plant atPAL (i.e., Arabidopsis thaliana PAL) and avPAL demonstrating comparable stability and biological activity out to the 120-hour time point and beyond.

These results show that both plant-derived PAL proteins and avPAL expressed from mRNA delivered via LNPs were biologically active in vivo.

### Discussion of Examples 1-2

Studies described herein demonstrate the capability of lipid-mediated delivery technology to deliver mRNA for a replacement enzyme, the bacterial phenylalanine ammonia lyase (avPAL), into hepatic tissue in vivo. The studies described herein further show that avPAL was capable of metabolizing Phe and reducing serum levels of Phe for more than five days post-transfection. Thus, avPAL delivered in vivo using LNPs remained active at clinically relevant levels for at least five days post-delivery. Without being limited by theory, once transfected into animals such as PKU mice, LNP-delivered PAL can fill the gap in the Phe metabolism pathway that results from PKU deficiency by facilitating the breakdown of Phe to tCA. The remaining steps of the metabolic pathway then complete the breakdown of tCA to HA.

The studies described herein further demonstrate the ability of lipid nanoparticles (LNPs) to deliver a plant-derived PAL protein with a similar effect on the level of serum Phe as compared to avPAL. Comparable transfection efficiencies were seen for bacterial avPAL and three different plant-derived PAL mRNAs. Intracellular mRNA stability and biological activity for at least one of the studied plant-derived PAL proteins was comparable to bacterial avPAL, as seen by reduction of serum Phe and the increase of serum HA (FIGS. 2D, 2E). Without being limited by theory, these results show a comparable level of in vivo activity of bacterial avPAL and plant-derived PAL, an important consideration in view of potentially reduced immunogenic side effects of plant-derived PAL proteins as compared to bacterial PAL.

Taken together, results provided herein demonstrate the effectiveness and usefulness of LNPs for the targeted delivery of PAL mRNA into hepatic tissue in vivo, resulting in functional replacement of a defective PAH protein and reduction of serum Phe levels, thereby ameliorating the underlying cause of PKU symptoms. Accordingly, LNP-mediated delivery of PAL represents a new and potentially effective treatment approach for PKU and related disorders. Advantageously, LNPs can accurately deliver their mRNA payload with low off-target effects (10). Studies described herein have shown that bacterial and plant-derived PAL mRNA is stable and that the expressed PAL protein can facilitate the breakdown of Phe that normally accumulates in patients suffering from PKU. PAL expressed from mRNA delivered via LNPs remained stable and biologically active in vivo for over five days, a sufficient duration for an effective and tolerable injection therapy. In addition, results described herein establish plant-based PAL proteins as a viable alternative to bacterial avPAL to reduce an immunologic response.

### REFERENCES

1. M. Gizewska, “Phenylketonuria: Phenylalanine Neurotoxicity” in Nutrition Management of Inherited Metabolic Diseases, L. Bernstein, F. Rohr, JR Helm, Eds. (Springer, 2015). p. 89-99
2. N. Blau, F. J. van Spronsen, H. L. Levy. Phenylketonuria. Lancet. 376, 1417-27 (2010).
3. D. Dobbelaere, L. Michaud, A. Debrabander, S. Vanderbecken, F. Gottrand, D. Turck, et al. Evaluation of nutritional status and pathophysiology of growth retardation in patients with phenylketonuria. J Inherit. Metab. Dis. 26, 1-11 (2003).
4. Kuvan® (sapropterin dihydrochloride) Tablets; Highlights of prescribing information, 2014. www.accessdata.fda.gov/drugsatfda_docs/label/2014/022181s0131bl.pdf
5. European Medicines Agency Kuvan: EPAR-Summary for the Public. www.ema.europa.eu/docs/en_GB/document_library/EPAR_-_Summary for the_public/human/000943/WC500045034.pdf
6. Barbara K. Burton, Heather Bausell, Rachel Katz, HollyLaDuca, Christine Sullivan. Sapropterin therapy increases stability of blood phenylalanine levels in patients with BH4-responsive phenylketonuria (PKU). Molecular Genetics and Metabolism. Volume 101, Issues 2-3, October-November 2010, Pages 110-114
7. Soumi Gupta, Kelly Lau, Cary O. Harding, Gillian Shepherd, Ryan Boyer, John P. Atkinson, Vijaya Knight, Joy Olbertz, Kevin Larimore, Zhonghu Gu, Mingjin Li, Orli Rosen, Stephen J. Zoog, Haoling H. Weng, Becky Schweighardt. Association of immune response with efficacy and safety outcomes in adults with phenylketonuria administered pegvaliase in phase 3 clinical trials. EBioMedicine, 37 (2018), pp. 366-373
8. FDA Drug Approval Package: PALYNZIQ (pegvaliase-pqpz). www.accessdata.fda.gov/drugsatfda_docs/nda/2018/761079Orig1s000Approv.pdf
9. N. Longo, D. Dimmock, H. Levy, K. Viau, H. Bausell, D. A. Bilder, B. Burton, C. Gross, H. Northrup, F. Rohr, et al. Evidence- and consensus-based recommendations for the use of pegvaliase in adults with phenylketonuria. Genet. Med., 21 (8) (2019), pp. 1851-1867
10. Ramaswamy S, Tonnu N, Tachikawa K, Limphong P, Vega J B, Karmali P P, Chivukula P, Verma I M. Systemic delivery of factor IX messenger RNA for protein replacement therapy. Proc Natl Acad Sci USA. 2017 Mar. 7; 114 (10):E1941-E1950.

SEQUENCES

SEQ ID NO: 1 avPAL ORF (with Myc and FLAG tags)
ATGGGCAAGACCCTGAGCCAGGCCCAGAGCAAGACCAGCAGCCAGCAGTTCAGCTTCACCGGCAACAGCAGCGCC
AACGTGATCATCGGCAACCAGAAGCTGACCATCAACGACGTGGCCAGGGTGGCCCGGAACGGCACCCTGGTGAGC
CTGACCAACAACACCGACATCCTGCAGGGCATCCAGGCCAGCTGCGACTACATCAACAACGCCGTGGAGAGCGGC
GAGCCCATCTACGGCGTGACCAGCGGCTTCGGCGGAATGGCCAACGTGGCCATCAGCAGGGAGCAGGCCAGCGAG
CTGCAGACCAACCTGGTGTGGTTCCTGAAGACCGGAGCCGGCAACAAGCTGCCACTGGCCGACGTGAGAGCAGCC
ATGCTCCTGAGGGCCAACAGCCACATGAGAGGCGCCAGCGGCATCAGGCTGGAGCTGATCAAGAGGATGGAGATC
TTCCTGAACGCCGGCGTGACCCCATACGTGTACGAGTTCGGCAGCATCGGCGCCAGCGGCGACCTGGTGCCCCTG
AGCTACATCACCGGCAGCCTGATCGGCCTGGACCCCAGCTTCAAGGTGGACTTCAACGGCAAGGAGATGGACGCC
CCAACCGCCCTGAGGCAGCTGAACCTGAGCCCCCTGACCCTGCTGCCCAAGGAGGGCCTGGCAATGATGAACGGC
ACCAGCGTGATGACCGGCATCGCCGCCAACTGCGTGTACGACACCCAGATCCTGACCGCCATCGCAATGGGCGTG
CACGCCCTGGACATCCAGGCCCTGAACGGCACCAACCAGAGCTTCCACCCCTTCATCCACAACAGCAAGCCACAC
CCCGGACAGCTGTGGGCCGCAGACCAGATGATCAGCCTGCTCGCCAACAGCCAGCTGGTGAGGGACGAGCTGGAC
GGCAAGCACGACTACAGGGACCACGAGCTGATCCAGGACAGGTACAGCCTGAGGTGCCTGCCCCAGTACCTGGGC
CCAATCGTGGACGGCATCAGCCAGATCGCCAAGCAGATCGAGATCGAGATCAACAGCGTGACCGACAACCCACTG
ATCGACGTGGACAACCAGGCCAGCTACCACGGCGGAAACTTCCTGGGCCAGTACGTGGGAATGGGCATGGACCAC
CTGAGGTACTACATCGGCCTGCTCGCCAAGCACCTGGACGTGCAGATCGCCCTGCTCGCCAGCCCAGAGTTCAGC
AACGGACTGCCACCCAGCCTCCTGGGCAACAGGGAGCGGAAGGTGAACATGGGCCTGAAGGGACTGCAGATCTGC
GGCAACAGCATCATGCCACTCCTGACCTTCTACGGCAACAGCATCGCCGACAGGTTCCCCACCCACGCCGAGCAG
TTCAACCAGAACATCAACAGCCAGGGCTACACCAGCGCCACCCTGGCCAGGCGGAGCGTGGACATCTTCCAGAAC
TACGTGGCCATCGCACTGATGTTCGGCGTGCAGGCCGTGGACCTGAGGACCTACAAGAAGACCGGCCACTACGAC
GCCAGGGCCTGCCTGAGCCCCGCCACCGAGAGGCTGTACAGCGCCGTGAGGCACGTGGTCGGCCAGAAGCCCACC
AGCGACAGGCCCTACATCTGGAACGACAACGAGCAGGGCCTGGACGAGCACATCGCCAGGATCAGCGCCGACATC
GCCGCAGGCGGAGTGATCGTGCAGGCCGTGCAGGACATCCTGCCCTGCCTGCACGCCCCCGCACCCGCCCCTAGG
GGAGGCGGGAGCGGCGAGCAGAAACTGATCAGCGAAGAGGACCTGGCCGCAAACGACATCCTGGACTACAAGGAT
GACGACGATAAGGTGTGA
SEQ ID NO: 2 avPAL ORF
ATGGGCAAGACCCTGAGCCAGGCCCAGAGCAAGACCAGCAGCCAGCAGTTCAGCTTCACCGGCAACAGCAGCGCC
AACGTGATCATCGGCAACCAGAAGCTGACCATCAACGACGTGGCCAGGGTGGCCCGGAACGGCACCCTGGTGAGC
CTGACCAACAACACCGACATCCTGCAGGGCATCCAGGCCAGCTGCGACTACATCAACAACGCCGTGGAGAGCGGC
GAGCCCATCTACGGCGTGACCAGCGGCTTCGGCGGAATGGCCAACGTGGCCATCAGCAGGGAGCAGGCCAGCGAG
CTGCAGACCAACCTGGTGTGGTTCCTGAAGACCGGAGCCGGCAACAAGCTGCCACTGGCCGACGTGAGAGCAGCC
ATGCTCCTGAGGGCCAACAGCCACATGAGAGGCGCCAGCGGCATCAGGCTGGAGCTGATCAAGAGGATGGAGATC
TTCCTGAACGCCGGCGTGACCCCATACGTGTACGAGTTCGGCAGCATCGGCGCCAGCGGCGACCTGGTGCCCCTG
AGCTACATCACCGGCAGCCTGATCGGCCTGGACCCCAGCTTCAAGGTGGACTTCAACGGCAAGGAGATGGACGCC
CCAACCGCCCTGAGGCAGCTGAACCTGAGCCCCCTGACCCTGCTGCCCAAGGAGGGCCTGGCAATGATGAACGGC
ACCAGCGTGATGACCGGCATCGCCGCCAACTGCGTGTACGACACCCAGATCCTGACCGCCATCGCAATGGGCGTG
CACGCCCTGGACATCCAGGCCCTGAACGGCACCAACCAGAGCTTCCACCCCTTCATCCACAACAGCAAGCCACAC
CCCGGACAGCTGTGGGCCGCAGACCAGATGATCAGCCTGCTCGCCAACAGCCAGCTGGTGAGGGACGAGCTGGAC
GGCAAGCACGACTACAGGGACCACGAGCTGATCCAGGACAGGTACAGCCTGAGGTGCCTGCCCCAGTACCTGGGC
CCAATCGTGGACGGCATCAGCCAGATCGCCAAGCAGATCGAGATCGAGATCAACAGCGTGACCGACAACCCACTG
ATCGACGTGGACAACCAGGCCAGCTACCACGGCGGAAACTTCCTGGGCCAGTACGTGGGAATGGGCATGGACCAC
CTGAGGTACTACATCGGCCTGCTCGCCAAGCACCTGGACGTGCAGATCGCCCTGCTCGCCAGCCCAGAGTTCAGC
AACGGACTGCCACCCAGCCTCCTGGGCAACAGGGAGCGGAAGGTGAACATGGGCCTGAAGGGACTGCAGATCTGC
GGCAACAGCATCATGCCACTCCTGACCTTCTACGGCAACAGCATCGCCGACAGGTTCCCCACCCACGCCGAGCAG
TTCAACCAGAACATCAACAGCCAGGGCTACACCAGCGCCACCCTGGCCAGGCGGAGCGTGGACATCTTCCAGAAC
TACGTGGCCATCGCACTGATGTTCGGCGTGCAGGCCGTGGACCTGAGGACCTACAAGAAGACCGGCCACTACGAC
GCCAGGGCCTGCCTGAGCCCCGCCACCGAGAGGCTGTACAGCGCCGTGAGGCACGTGGTCGGCCAGAAGCCCACC
AGCGACAGGCCCTACATCTGGAACGACAACGAGCAGGGCCTGGACGAGCACATCGCCAGGATCAGCGCCGACATC
GCCGCAGGCGGAGTGATCGTGCAGGCCGTGCAGGACATCCTGCCCTGCCTGCACGCCCCCGCACCCGCCCCTAGG
SEQ ID NO: 3 mutant avPAL ORF (with Myc and FLAG tags)
ATGGGCAAGACCCTGAGCCAGGCCCAGAGCAAGACCAGCAGCCAGCAGTTCAGCTTCACCGGCAACAGCAGCGCC
AACGTGATCATCGGCAACCAGAAGCTGACCATCAACGACGTGGCCAGGGTGGCCCGGAACGGCACCCTGGTGAGC
CTGACCAACAACACCGACATCCTGCAGGGCATCCAGGCCAGCTGCGACTACATCAACAACGCCGTGGAGAGCGGC
GAGCCCATCTACGGCGTGACCAGCGGCTTCGGCGGAATGGCCAACGTGGCCATCAGCAGGGAGCAGGCCAGCGAG
CTGCAGACCAACCTGGTGTGGTTCCTGAAGACCGGAGCCGGCAACAAGCTGCCACTGGCCGACGTGAGAGCAGCC
ATGCTCCTGAGGGCCAACAGCCACATGAGAGGCGCCAGCGGCATCAGGCTGGAGCTGATCAAGAGGATGGAGATC
TTCCTGAACGCCGGCGTGACCCCATACGTGTACGAGTTCGGCAGCATCGGCGCCAGCGGCGACCTGGTGCCCCTG
AGCTACATCACCGGCAGCCTGATCGGCCTGGACCCCAGCTTCAAGGTGGACTTCAACGGCAAGGAGATGGACGCC
CCAACCGCCCTGAGGCAGCTGAACCTGAGCCCCCTGACCCTGCTGCCCAAGGAGGGCCTGGCAATGATGAACGGC
ACCAGCGTGATGACCGGCATCGCCGCCAACTGCGTGTACGACACCCAGATCCTGACCGCCATCGCAATGGGCGTG
CACGCCCTGGACATCCAGGCCCTGAACGGCACCAACCAGAGCTTCCACCCCTTCATCCACAACAGCAAGCCACAC
CCCGGACAGCTGTGGGCCGCAGACCAGATGATCAGCCTGCTCGCCAACAGCCAGCTGGTGAGGGACGAGCTGGAC
GGCAAGCACGACTACAGGGACCACGAGCTGATCCAGGACAGGTACAGCCTGAGGTGCCTGCCCCAGTACCTGGGC
CCAATCGTGGACGGCATCAGCCAGATCGCCAAGCAGATCGAGATCGAGATCAACAGCGTGACCGACAACCCACTG
ATCGACGTGGACAACCAGGCCAGCTACCACGGCGGAAACTTCCTGGGCCAGTACGTGGGAATGGGCATGGACCAC
CTGAGGTACTACATCGGCCTGCTCGCCAAGCACCTGGACGTGCAGATCGCCCTGCTCGCCAGCCCAGAGTTCAGC
AACGGACTGCCACCCAGCCTCCTGGGCAACAGGGAGCGGAAGGTGAACATGGGCCTGAAGGGACTGCAGATCTGC
GGCAACAGCATCATGCCACTCCTGACCTTCTACGGCAACAGCATCGCCGACAGGTTCCCCACCCACGCCGAGCAG
TTCAACCAGAACATCAACAGCCAGGGCTACACCAGCGCCACCCTGGCCAGGCGGAGCGTGGACATCTTCCAGAAC
TACGTGGCCATCGCACTGATGTTCGGCGTGCAGGCCGTGGACCTGAGGACCTACAAGAAGACCGGCCACTACGAC
GCCAGGGCCAGCCTGAGCCCCGCCACCGAGAGGCTGTACAGCGCCGTGAGGCACGTGGTCGGCCAGAAGCCCACC
AGCGACAGGCCCTACATCTGGAACGACAACGAGCAGGGCCTGGACGAGCACATCGCCAGGATCAGCGCCGACATC
GCCGCAGGCGGAGTGATCGTGCAGGCCGTGCAGGACATCCTGCCCAGCCTGCACGCCCCCGCACCCGCCCCTAGG
GGAGGCGGGAGCGGCGAGCAGAAACTGATCAGCGAAGAGGACCTGGCCGCAAACGACATCCTGGACTACAAGGAT
GACGACGATAAGGTGT
SEQ ID NO: 4 mutant avPAL ORF
ATGGGCAAGACCCTGAGCCAGGCCCAGAGCAAGACCAGCAGCCAGCAGTTCAGCTTCACCGGCAACAGCAGCGCC
AACGTGATCATCGGCAACCAGAAGCTGACCATCAACGACGTGGCCAGGGTGGCCCGGAACGGCACCCTGGTGAGC
CTGACCAACAACACCGACATCCTGCAGGGCATCCAGGCCAGCTGCGACTACATCAACAACGCCGTGGAGAGCGGC
GAGCCCATCTACGGCGTGACCAGCGGCTTCGGCGGAATGGCCAACGTGGCCATCAGCAGGGAGCAGGCCAGCGAG
CTGCAGACCAACCTGGTGTGGTTCCTGAAGACCGGAGCCGGCAACAAGCTGCCACTGGCCGACGTGAGAGCAGCC
ATGCTCCTGAGGGCCAACAGCCACATGAGAGGCGCCAGCGGCATCAGGCTGGAGCTGATCAAGAGGATGGAGATC
TTCCTGAACGCCGGCGTGACCCCATACGTGTACGAGTTCGGCAGCATCGGCGCCAGCGGCGACCTGGTGCCCCTG
AGCTACATCACCGGCAGCCTGATCGGCCTGGACCCCAGCTTCAAGGTGGACTTCAACGGCAAGGAGATGGACGCC
CCAACCGCCCTGAGGCAGCTGAACCTGAGCCCCCTGACCCTGCTGCCCAAGGAGGGCCTGGCAATGATGAACGGC
ACCAGCGTGATGACCGGCATCGCCGCCAACTGCGTGTACGACACCCAGATCCTGACCGCCATCGCAATGGGCGTG
CACGCCCTGGACATCCAGGCCCTGAACGGCACCAACCAGAGCTTCCACCCCTTCATCCACAACAGCAAGCCACAC
CCCGGACAGCTGTGGGCCGCAGACCAGATGATCAGCCTGCTCGCCAACAGCCAGCTGGTGAGGGACGAGCTGGAC
GGCAAGCACGACTACAGGGACCACGAGCTGATCCAGGACAGGTACAGCCTGAGGTGCCTGCCCCAGTACCTGGGC
CCAATCGTGGACGGCATCAGCCAGATCGCCAAGCAGATCGAGATCGAGATCAACAGCGTGACCGACAACCCACTG
ATCGACGTGGACAACCAGGCCAGCTACCACGGCGGAAACTTCCTGGGCCAGTACGTGGGAATGGGCATGGACCAC
CTGAGGTACTACATCGGCCTGCTCGCCAAGCACCTGGACGTGCAGATCGCCCTGCTCGCCAGCCCAGAGTTCAGC
AACGGACTGCCACCCAGCCTCCTGGGCAACAGGGAGCGGAAGGTGAACATGGGCCTGAAGGGACTGCAGATCTGC
GGCAACAGCATCATGCCACTCCTGACCTTCTACGGCAACAGCATCGCCGACAGGTTCCCCACCCACGCCGAGCAG
TTCAACCAGAACATCAACAGCCAGGGCTACACCAGCGCCACCCTGGCCAGGCGGAGCGTGGACATCTTCCAGAAC
TACGTGGCCATCGCACTGATGTTCGGCGTGCAGGCCGTGGACCTGAGGACCTACAAGAAGACCGGCCACTACGAC
GCCAGGGCCAGCCTGAGCCCCGCCACCGAGAGGCTGTACAGCGCCGTGAGGCACGTGGTCGGCCAGAAGCCCACC
AGCGACAGGCCCTACATCTGGAACGACAACGAGCAGGGCCTGGACGAGCACATCGCCAGGATCAGCGCCGACATC
GCCGCAGGCGGAGTGATCGTGCAGGCCGTGCAGGACATCCTGCCCAGCCTGCACGCCCCCGCACCCGCCCCTAGG
SEQ ID NO: 5 Arabidopsis thaliana PAL ORF
ATGGACCAGATTGAGGCCATGCTGTGCGGCGGCGGCGAGAAGACCAAGGTGGCCGTGACAACCAAGACCCTGGCC
GACCCTCTGAACTGGGGCCTGGCCGCCGACCAGATGAAGGGCAGCCACCTGGACGAGGTGAAGAAGATGGTGGAG
GAGTACAGGAGGCCCGTGGTGAACCTGGGCGGCGAGACACTGACCATCGGCCAGGTGGCCGCCATCAGCACCGTG
GGCGGCAGCGTGAAGGTGGAGCTGGCCGAGACAAGCAGGGCCGGCGTGAAGGCCAGCAGCGACTGGGTGATGGAG
AGCATGAACAAGGGCACCGACAGCTACGGCGTGACCACCGGCTTCGGCGCCACCAGCCACCGGAGGACCAAGAAC
GGCACCGCCCTGCAGACCGAGCTGATCAGGTTCCTGAACGCCGGCATCTTCGGCAACACCAAGGAGACATGCCAC
ACCCTGCCCCAGAGCGCCACCAGGGCCGCCATGCTGGTGAGGGTGAACACCCTGCTGCAGGGCTACAGCGGCATC
AGGTTCGAGATCCTGGAGGCCATCACCAGCCTGCTGAACCACAACATCAGCCCCAGCCTGCCCCTGAGGGGCACC
ATCACCGCCAGCGGCGACCTGGTGCCCCTGAGCTACATCGCCGGCCTGCTGACCGGCAGGCCCAACAGCAAGGCC
ACCGGCCCCGACGGCGAGAGCCTGACCGCCAAGGAGGCCTTCGAGAAGGCCGGCATCAGCACCGGCTTCTTCGAC
CTGCAGCCCAAGGAGGGCCTGGCCCTGGTGAACGGCACCGCCGTGGGCAGCGGCATGGCCAGCATGGTGCTGTTC
GAGGCCAACGTGCAGGCCGTGCTGGCCGAGGTGCTGAGCGCCATCTTCGCCGAGGTGATGAGCGGCAAGCCCGAG
TTCACCGACCACCTGACCCACAGGCTGAAGCACCACCCCGGCCAGATCGAGGCCGCCGCCATCATGGAGCACATC
CTGGACGGCAGCAGCTACATGAAGCTGGCCCAGAAGGTGCACGAGATGGACCCTCTGCAGAAGCCCAAGCAGGAC
AGGTACGCCCTGAGGACCAGCCCTCAGTGGCTGGGCCCTCAGATCGAGGTGATCAGGCAGGCCACCAAGAGCATC
GAGAGGGAGATCAACAGCGTGAACGACAATCCCCTGATCGACGTGAGCAGGAACAAGGCCATCCACGGCGGCAAC
TTCCAGGGCACCCCTATCGGCGTGAGCATGGACAACACCAGGCTGGCCATCGCCGCCATCGGCAAGCTGATGTTC
GCCCAGTTCAGCGAGCTGGTGAACGACTTCTACAACAACGGCCTGCCCAGCAACCTGACCGCCAGCAGCAACCCC
AGCCTGGACTACGGCTTCAAGGGCGCCGAGATCGCTATGGCCAGCTACTGCAGCGAGCTGCAGTACCTGGCCAAC
CCCGTGACCAGCCACGTGCAGAGCGCCGAGCAGCACAACCAGGACGTGAACAGCCTGGGCCTGATCAGCAGCAGG
AAGACCAGCGAGGCCGTGGACATCCTGAAGCTGATGAGCACCACCTTCCTGGTGGGCATCTGCCAGGCCGTGGAC
CTGAGGCACCTGGAGGAGAACCTGAGGCAGACCGTGAAGAACACCGTGAGCCAGGTGGCCAAGAAGGTGCTGACC
ACCGGCATCAACGGCGAGCTGCACCCCAGCAGGTTCTGCGAGAAGGACCTGCTGAAGGTGGTGGACAGGGAGCAG
GTGTTCACCTACGTGGACGACCCCTGCAGCGCCACCTACCCTCTGATGCAGAGGCTGAGGCAGGTGATCGTGGAC
CACGCCCTGAGCAACGGCGAGACAGAGAAGAACGCCGTGACCAGCATCTTCCAGAAGATCGGCGCCTTCGAGGAG
GAGCTGAAGGCCGTGCTGCCCAAGGAGGTGGAGGCCGCCAGGGCCGCCTACGGCAACGGCACCGCCCCTATCCCC
AACCGGATCAAGGAGTGCAGGAGCTACCCTCTGTACCGGTTCGTGAGGGAGGAGCTGGGCACCAAGCTGCTGACC
GGCGAGAAGGTGGTGAGCCCCGGCGAGGAGTTCGACAAGGTGTTCACCGCCATGTGCGAGGGCAAGCTGATCGAC
CCTCTGATGGACTGCCTGAAGGAGTGGAACGGCGCCCCTATCCCCATCTGCCCTAGGGGAGGCGGGAGCGGCGAG
CAGAAACTGATCAGCGAAGAGGACCTGGCCGCAAACGACATCCTGGACTACAAGGACGACGACGACAAGTAG
SEQ ID NO: 6 Solanum lycopersicum PAL ORF
ATGGCCTCTAGCATCGTGCAGAACGGCCACGTGAATGGCGAGGCTATGGACCTGTGCAAGAAGTCCATCAACGTG
AACGACCCTCTGAACTGGGAGATGGCCGCCGAGAGCCTGAGGGGCAGCCACCTGGACGAGGTGAAGAAGATGGTG
GACGAGTTCAGGAAGCCCATCGTGAAGCTGGGCGGCGAGACACTGACCGTGGCCCAGGTGGCCAGCATCGCCAAC
GTGGACAACAAGAGCAACGGCGTGAAGGTGGAGCTGAGCGAGAGCGCCAGGGCCGGCGTGAAGGCCAGCAGCGAC
TGGGTGATGGACAGCATGGGCAAGGGCACCGACAGCTACGGCGTGACCACCGGCTTCGGCGCCACCAGCCACAGG
AGGACCAAGAACGGCGGCGCCCTGCAGAAGGAGCTGATCAGGTTCCTGAACGCCGGCGTGTTCGGCAACGGCACC
GAGAGCAGCCACACCCTGCCCCACAGCGCCACCAGGGCCGCCATGCTGGTGAGGATCAACACCCTGCTGCAGGGC
TACAGCGGCATCAGGTTCGAGATCCTGGAGGCCATCACCAAGCTGATCAACAGCAACATCACCCCTTGCCTGCCC
CTGAGGGGCACCATCACCGCCAGCGGCGACCTGGTGCCCCTGAGCTACATCGCCGGCCTGCTGACCGGCAGGCCC
AACAGCAAGGCCGTGGGCCCCAACGGCGAGAAGCTGAACGCCGAGGAGGCCTTCCACGTGGCCGGCGTGACCAGC
GGCTTCTTCGAGCTGCAGCCCAAGGAGGGCCTGGCCCTGGTGAACGGCACCGCCGTGGGCAGCGGCATGGCCAGC
ATGGTGCTGTTCGAGAGCAACATCCTGGCCGTGATGAGCGAGGTGCTGAGCGCCATCTTCGCCGAGGTGATGAAC
GGCAAGCCCGAGTTCACCGACTACCTGACCCACAAGCTGAAGCACCACCCCGGCCAGATCGAGGCCGCCGCCATC
ATGGAGCACATCCTGGACGGCAGCAGCTACGTGAAGGAGGCCCAGAAGCTGCACGAGATGGACCCTCTGCAGAAG
CCCAAGCAGGACAGGTACGCCCTGAGGACCAGCCCTCAGTGGCTGGGCCCTCAGATCGAGGTGATCAGGGCCGCC
ACCAAGATGATCGAGAGGGAGATCAACAGCGTGAACGACAATCCCCTGATCGACGTGAGCAGGAACAAGGCCCTG
CACGGCGGCAACTTCCAGGGCACCCCTATCGGCGTGAGCATGGACAACACCAGGCTGGCCCTGGCCAGCATCGGC
AAGCTGATGTTCGCCCAGTTCAGCGAGCTGGTGAACGACTACTACAACAACGGCCTGCCCAGCAACCTGACCGCC
GGCAGGAACCCCAGCCTGGACTACGGCTTCAAGGGCGCCGAGATCGCTATGGCCAGCTACTGCAGCGAGCTGCAG
TTCCTGGCCAACCCCGTGACCAACCACGTGCAGAGCGCCGAGCAGCACAACCAGGACGTGAACAGCCTGGGCCTG
ATCAGCGCCAGGAAGACCGCCGAGGCCGTGGACATCCTGAAGCTGATGAGCAGCACCTACCTGGTGGCCCTGTGC
CAGGCCATCGACCTGAGGCACCTGGAGGAGAACCTGAAGAACGCCGTGAAGAACACCGTGAGCCAGGTGGCCAAG
AAGACCCTGGCTATGGGCGCCAACGGCGAGCTGCACCCCGCCAGGTTCTGCGAGAAGGAGCTGCTGCAGGTGGTG
GAGAGGGAGTACCTGTTCACCTACGCCGACGACCCCTGCAGCAGCACCTACCCTCTGATGCAGAAGCTGAGGCAG
GTGCTGGTGGACCACGCCATGAAGAACGGCGAGAGCGAGAAGAACGTGAACAGCAGCATCTTCCAGAAGATCGTG
GCCTTCGAGGACGAGCTGAAGGCCGTGCTGCCCAAGGAGGTGGAGAGCGCCAGGGCCGTGGTGGAGAGCGGCAAC
CCCGCCATCCCCAACAGGATCACCGAGTGCAGGAGCTACCCTCTGTACCGGCTGGTGAGGCAGGAGGTGGGCACC
GAGCTGCTGACCGGCGAGAAGGTGAGGAGCCCCGGCGAGGAGATCGACAAGGTGTTCACCGCCTTCTGCAACGGC
CAGATCATCGACCCTCTGCTGGAGTGCCTGAAGTCCTGGAACGGCGCCCCTATCCCCATCTGCCCTAGGGGAGGC
GGGAGCGGCGAGCAGAAACTGATCAGCGAAGAGGACCTGGCCGCAAACGACATCCTGGACTACAAGGACGACGAC
GACAAGTAG
SEQ ID NO: 7 Nicotiana tabacum PAL ORF
ATGGCCGGCGTGGCCCAGAACGGCCACCAGGAGATGGACTTCTGCGTTAAGGTGGACCCTCTGAACTGGGAGATG
GCCGCCGACAGCCTGAAGGGCAGCCACCTGGACGAGGTGAAGAAGATGGTGGCCGAGTTCAGGAAGCCCGTGGTG
AAGCTGGGCGGCGAGACACTGACCGTGGCCCAGGTGGCCGCCATCGCCGCCAAGGACAACGCCAAGACCGTGAAG
GTGGAGCTGAGCGAGGGCGCCAGGGCCGGCGTGAAGGCCAGCAGCGACTGGGTGATGGACAGCATGAGCAAGGGC
ACCGACAGCTACGGCGTGACCACCGGCTTCGGCGCCACCAGCCACAGGAGGACCAAGAACGGCGGCGCCCTGCAG
AAGGAGCTGATCAGGTTCCTGAACGCCGGCGTGTTCGGCAACGGCACCGAGAGCTGCCACACCCTGCCCCAGAGC
GGCACCAGGGCCGCCATGCTGGTGAGGATCAACACCCTGCTGCAGGGCTACAGCGGCATCAGGTTCGAGATCCTG
GAGGCCATCACCAAGCTGCTGAACCACAACGTGACCCCTTGCCTGCCCCTGAGGGGCACCATCACCGCCAGCGGC
GACCTGGTGCCCCTGAGCTACATCGCCGGCCTGCTGACCGGCCGGCCCAACAGCAAGGCCATCGGCCCCAACGGC
GAGACACTGAACGCCGAGGAGGCCTTCAGGGTGGCCGGCGTGAACAGCGGCTTCTTCGAGCTGCAGCCCAAGGAG
GGCCTGGCCCTGGTGAACGGCACCGCCGTGGGCAGCGGCCTGGCCAGCATGGTGCTGTTCGACGCCAACATCCTG
GCCGTGTTCAGCGAGGTGCTGAGCGCCATCTTCGCCGAGGTGATGAACGGCAAGCCCGAGTTCACCGACCACCTG
ACCCACAAGCTGAAGCACCACCCCGGCCAGATCGAGGCCGCCGCCATCATGGAGCACATCCTGGACGGCAGCAGC
TACGTGAAGGCCCCTCAGAAGCTGCACGAGACAGACCCTCTGCAGAAGCCCAAGCAGGACAGGTACGCCCTGAGG
ACCAGCCCTCAGTGGCTGGGCCCTCAGATCGAGGTGATCAGGAGCGCCACCAAGATGATCGAGAGGGAGATCAAC
AGCGTGAACGACAATCCCCTGATCGACGTGAGCAGGAACAAGGCCCTGCACGGCGGCAACTTCCAGGGCACCCCT
ATCGGCGTGAGCATGGACAACGCCAGGCTGGCCCTGGCCAGCATCGGCAAGCTGATGTTCGCCCAGTTCAGCGAG
CTGGTGAACGACTACTACAACAACGGCCTGCCCAGCAACCTGACCGCCGGCAGGAACCCCAGCCTGGACTACGGC
TTCAAGGGCAGCGAGATCGCTATGGCCAGCTACTGCAGCGAGCTGCAGTTCCTGGCCAACCCCGTGACCAACCAC
GTGCAGAGCGCCGAGCAGCACAACCAGGACGTGAACAGCCTGGGCCTGATCAGCGCCAGGAAGACCGCCGAGGCC
GTGGACATCCTGAAGCTGATGAGCAGCACCTACCTGGTGGCCCTGTGCCAGGCCATCGACCTGAGGCACCTGGAG
GAGAACCTGAGGAACGCCGTGAAGAACACCGTGAGCCAGGTGGCCAAGAGGACCCTGACAATGGGCGCCAACGGC
GAGCTGCACCCCAGCAGGTTCTGCGAGAAGGACCTGCTGAGGGTGGTGGACAGGGAGTACGTGTTCAGGTACGCC
GACGACGCCTGCAGCGCCAACTACCCTCTGATGCAGAAGCTGAGGCAGGTGCTGGTGGACCACGCCCTGGAGAAC
GGCGAGAACGAGAAGAACGCCAACAGCAGCATCTTCCAGAAGATCCTGGCCTTCGAGGGCGAGCTGAAGGCCGTG
CTGCCCAAGGAGGTGGAGAGCGCCAGGATCAGCCTGGAGAACGGCAACCCCGCCATCGCCAACAGGATCAAGGAG
TGCAGGAGCTACCCTCTGTACCGGTTCGTGAGGGAGGAGCTGGGCGCCGAGCTGCTGACCGGCGAGAAGGTGAGG
AGCCCCGGCGAGGAGTGCGACAAGGTGTTCACCGCCATGTGCAACGGCCAGATCATCGACAGCCTGCTGGAGTGC
CTGAAGGAGTGGAACGGCGCCCCTCTGCCCATCTGCCCTAGGGGAGGCGGGAGCGGCGAGCAGAAACTGATCAGC
GAAGAGGACCTGGCCGCAAACGACATCCTGGACTACAAGGACGACGACGACAAGTAG
SEQ ID NO: 8 TEV 5′ UTR
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACC
SEQ ID NO: 9 XBG 3′ UTR
TCGAGCTAGTGACTGACTAGGATCTGGTTACCACTAAACCAGCCTCAAGAACACCCGAATGGAGTCTCTAAGCTA
CATAATACCAACTTACACTTACAAAATGTTGTCCCCCAAAATGTAGCCATTCGTATCTGCTCCTAATAAAAAGAA
AGTTTCTTCACATTCTAG
SEQ ID NO: 10 avPAL complete mRNA sequence (with FLAG and Myc tags)
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACCATGG
GCAAGACCCTGAGCCAGGCCCAGAGCAAGACCAGCAGCCAGCAGTTCAGCTTCACCGGCAACAGCAGCGCCAACG
TGATCATCGGCAACCAGAAGCTGACCATCAACGACGTGGCCAGGGTGGCCCGGAACGGCACCCTGGTGAGCCTGA
CCAACAACACCGACATCCTGCAGGGCATCCAGGCCAGCTGCGACTACATCAACAACGCCGTGGAGAGCGGCGAGC
CCATCTACGGCGTGACCAGCGGCTTCGGCGGAATGGCCAACGTGGCCATCAGCAGGGAGCAGGCCAGCGAGCTGC
AGACCAACCTGGTGTGGTTCCTGAAGACCGGAGCCGGCAACAAGCTGCCACTGGCCGACGTGAGAGCAGCCATGC
TCCTGAGGGCCAACAGCCACATGAGAGGCGCCAGCGGCATCAGGCTGGAGCTGATCAAGAGGATGGAGATCTTCC
TGAACGCCGGCGTGACCCCATACGTGTACGAGTTCGGCAGCATCGGCGCCAGCGGCGACCTGGTGCCCCTGAGCT
ACATCACCGGCAGCCTGATCGGCCTGGACCCCAGCTTCAAGGTGGACTTCAACGGCAAGGAGATGGACGCCCCAA
CCGCCCTGAGGCAGCTGAACCTGAGCCCCCTGACCCTGCTGCCCAAGGAGGGCCTGGCAATGATGAACGGCACCA
GCGTGATGACCGGCATCGCCGCCAACTGCGTGTACGACACCCAGATCCTGACCGCCATCGCAATGGGCGTGCACG
CCCTGGACATCCAGGCCCTGAACGGCACCAACCAGAGCTTCCACCCCTTCATCCACAACAGCAAGCCACACCCCG
GACAGCTGTGGGCCGCAGACCAGATGATCAGCCTGCTCGCCAACAGCCAGCTGGTGAGGGACGAGCTGGACGGCA
AGCACGACTACAGGGACCACGAGCTGATCCAGGACAGGTACAGCCTGAGGTGCCTGCCCCAGTACCTGGGCCCAA
TCGTGGACGGCATCAGCCAGATCGCCAAGCAGATCGAGATCGAGATCAACAGCGTGACCGACAACCCACTGATCG
ACGTGGACAACCAGGCCAGCTACCACGGCGGAAACTTCCTGGGCCAGTACGTGGGAATGGGCATGGACCACCTGA
GGTACTACATCGGCCTGCTCGCCAAGCACCTGGACGTGCAGATCGCCCTGCTCGCCAGCCCAGAGTTCAGCAACG
GACTGCCACCCAGCCTCCTGGGCAACAGGGAGCGGAAGGTGAACATGGGCCTGAAGGGACTGCAGATCTGCGGCA
ACAGCATCATGCCACTCCTGACCTTCTACGGCAACAGCATCGCCGACAGGTTCCCCACCCACGCCGAGCAGTTCA
ACCAGAACATCAACAGCCAGGGCTACACCAGCGCCACCCTGGCCAGGCGGAGCGTGGACATCTTCCAGAACTACG
TGGCCATCGCACTGATGTTCGGCGTGCAGGCCGTGGACCTGAGGACCTACAAGAAGACCGGCCACTACGACGCCA
GGGCCTGCCTGAGCCCCGCCACCGAGAGGCTGTACAGCGCCGTGAGGCACGTGGTCGGCCAGAAGCCCACCAGCG
ACAGGCCCTACATCTGGAACGACAACGAGCAGGGCCTGGACGAGCACATCGCCAGGATCAGCGCCGACATCGCCG
CAGGCGGAGTGATCGTGCAGGCCGTGCAGGACATCCTGCCCTGCCTGCACGCCCCCGCACCCGCCCCTAGGGGAG
GCGGGAGCGGCGAGCAGAAACTGATCAGCGAAGAGGACCTGGCCGCAAACGACATCCTGGACTACAAGGATGACG
ACGATAAGGTGTGACTCGAGCTAGTGACTGACTAGGATCTGGTTACCACTAAACCAGCCTCAAGAACACCCGAAT
GGAGTCTCTAAGCTACATAATACCAACTTACACTTACAAAATGTTGTCCCCCAAAATGTAGCCATTCGTATCTGC
TCCTAATAAAAAGAAAGTTTCTTCACATTCTAGAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAA
SEQ ID NO: 11 avPAL complete mRNA sequence
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACCATGG
GCAAGACCCTGAGCCAGGCCCAGAGCAAGACCAGCAGCCAGCAGTTCAGCTTCACCGGCAACAGCAGCGCCAACG
TGATCATCGGCAACCAGAAGCTGACCATCAACGACGTGGCCAGGGTGGCCCGGAACGGCACCCTGGTGAGCCTGA
CCAACAACACCGACATCCTGCAGGGCATCCAGGCCAGCTGCGACTACATCAACAACGCCGTGGAGAGCGGCGAGC
CCATCTACGGCGTGACCAGCGGCTTCGGCGGAATGGCCAACGTGGCCATCAGCAGGGAGCAGGCCAGCGAGCTGC
AGACCAACCTGGTGTGGTTCCTGAAGACCGGAGCCGGCAACAAGCTGCCACTGGCCGACGTGAGAGCAGCCATGC
TCCTGAGGGCCAACAGCCACATGAGAGGCGCCAGCGGCATCAGGCTGGAGCTGATCAAGAGGATGGAGATCTTCC
TGAACGCCGGCGTGACCCCATACGTGTACGAGTTCGGCAGCATCGGCGCCAGCGGCGACCTGGTGCCCCTGAGCT
ACATCACCGGCAGCCTGATCGGCCTGGACCCCAGCTTCAAGGTGGACTTCAACGGCAAGGAGATGGACGCCCCAA
CCGCCCTGAGGCAGCTGAACCTGAGCCCCCTGACCCTGCTGCCCAAGGAGGGCCTGGCAATGATGAACGGCACCA
GCGTGATGACCGGCATCGCCGCCAACTGCGTGTACGACACCCAGATCCTGACCGCCATCGCAATGGGCGTGCACG
CCCTGGACATCCAGGCCCTGAACGGCACCAACCAGAGCTTCCACCCCTTCATCCACAACAGCAAGCCACACCCCG
GACAGCTGTGGGCCGCAGACCAGATGATCAGCCTGCTCGCCAACAGCCAGCTGGTGAGGGACGAGCTGGACGGCA
AGCACGACTACAGGGACCACGAGCTGATCCAGGACAGGTACAGCCTGAGGTGCCTGCCCCAGTACCTGGGCCCAA
TCGTGGACGGCATCAGCCAGATCGCCAAGCAGATCGAGATCGAGATCAACAGCGTGACCGACAACCCACTGATCG
ACGTGGACAACCAGGCCAGCTACCACGGCGGAAACTTCCTGGGCCAGTACGTGGGAATGGGCATGGACCACCTGA
GGTACTACATCGGCCTGCTCGCCAAGCACCTGGACGTGCAGATCGCCCTGCTCGCCAGCCCAGAGTTCAGCAACG
GACTGCCACCCAGCCTCCTGGGCAACAGGGAGCGGAAGGTGAACATGGGCCTGAAGGGACTGCAGATCTGCGGCA
ACAGCATCATGCCACTCCTGACCTTCTACGGCAACAGCATCGCCGACAGGTTCCCCACCCACGCCGAGCAGTTCA
ACCAGAACATCAACAGCCAGGGCTACACCAGCGCCACCCTGGCCAGGCGGAGCGTGGACATCTTCCAGAACTACG
TGGCCATCGCACTGATGTTCGGCGTGCAGGCCGTGGACCTGAGGACCTACAAGAAGACCGGCCACTACGACGCCA
GGGCCTGCCTGAGCCCCGCCACCGAGAGGCTGTACAGCGCCGTGAGGCACGTGGTCGGCCAGAAGCCCACCAGCG
ACAGGCCCTACATCTGGAACGACAACGAGCAGGGCCTGGACGAGCACATCGCCAGGATCAGCGCCGACATCGCCG
CAGGCGGAGTGATCGTGCAGGCCGTGCAGGACATCCTGCCCTGCCTGCACGCCCCCGCACCCGCCCCTAGGGGAG
GCGGGAGCGGCGAGCAGAAACTGATCAGCGAAGAGGACCTGGCCGCAAACGACATCCTGGACTACAAGGATGACG
ACGATAAGGTGTGACTCGAGCTAGTGACTGACTAGGATCTGGTTACCACTAAACCAGCCTCAAGAACACCCGAAT
GGAGTCTCTAAGCTACATAATACCAACTTACACTTACAAAATGTTGTCCCCCAAAATGTAGCCATTCGTATCTGC
TCCTAATAAAAAGAAAGTTTCTTCACATTCTAGAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAA
SEQ ID NO: 12 mutant avPAL complete mRNA sequence (with FLAG and Myc tags)
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACCATGG
GCAAGACCCTGAGCCAGGCCCAGAGCAAGACCAGCAGCCAGCAGTTCAGCTTCACCGGCAACAGCAGCGCCAACG
TGATCATCGGCAACCAGAAGCTGACCATCAACGACGTGGCCAGGGTGGCCCGGAACGGCACCCTGGTGAGCCTGA
CCAACAACACCGACATCCTGCAGGGCATCCAGGCCAGCTGCGACTACATCAACAACGCCGTGGAGAGCGGCGAGC
CCATCTACGGCGTGACCAGCGGCTTCGGCGGAATGGCCAACGTGGCCATCAGCAGGGAGCAGGCCAGCGAGCTGC
AGACCAACCTGGTGTGGTTCCTGAAGACCGGAGCCGGCAACAAGCTGCCACTGGCCGACGTGAGAGCAGCCATGC
TCCTGAGGGCCAACAGCCACATGAGAGGCGCCAGCGGCATCAGGCTGGAGCTGATCAAGAGGATGGAGATCTTCC
TGAACGCCGGCGTGACCCCATACGTGTACGAGTTCGGCAGCATCGGCGCCAGCGGCGACCTGGTGCCCCTGAGCT
ACATCACCGGCAGCCTGATCGGCCTGGACCCCAGCTTCAAGGTGGACTTCAACGGCAAGGAGATGGACGCCCCAA
CCGCCCTGAGGCAGCTGAACCTGAGCCCCCTGACCCTGCTGCCCAAGGAGGGCCTGGCAATGATGAACGGCACCA
GCGTGATGACCGGCATCGCCGCCAACTGCGTGTACGACACCCAGATCCTGACCGCCATCGCAATGGGCGTGCACG
CCCTGGACATCCAGGCCCTGAACGGCACCAACCAGAGCTTCCACCCCTTCATCCACAACAGCAAGCCACACCCCG
GACAGCTGTGGGCCGCAGACCAGATGATCAGCCTGCTCGCCAACAGCCAGCTGGTGAGGGACGAGCTGGACGGCA
AGCACGACTACAGGGACCACGAGCTGATCCAGGACAGGTACAGCCTGAGGTGCCTGCCCCAGTACCTGGGCCCAA
TCGTGGACGGCATCAGCCAGATCGCCAAGCAGATCGAGATCGAGATCAACAGCGTGACCGACAACCCACTGATCG
ACGTGGACAACCAGGCCAGCTACCACGGCGGAAACTTCCTGGGCCAGTACGTGGGAATGGGCATGGACCACCTGA
GGTACTACATCGGCCTGCTCGCCAAGCACCTGGACGTGCAGATCGCCCTGCTCGCCAGCCCAGAGTTCAGCAACG
GACTGCCACCCAGCCTCCTGGGCAACAGGGAGCGGAAGGTGAACATGGGCCTGAAGGGACTGCAGATCTGCGGCA
ACAGCATCATGCCACTCCTGACCTTCTACGGCAACAGCATCGCCGACAGGTTCCCCACCCACGCCGAGCAGTTCA
ACCAGAACATCAACAGCCAGGGCTACACCAGCGCCACCCTGGCCAGGCGGAGCGTGGACATCTTCCAGAACTACG
TGGCCATCGCACTGATGTTCGGCGTGCAGGCCGTGGACCTGAGGACCTACAAGAAGACCGGCCACTACGACGCCA
GGGCCAGCCTGAGCCCCGCCACCGAGAGGCTGTACAGCGCCGTGAGGCACGTGGTCGGCCAGAAGCCCACCAGCG
ACAGGCCCTACATCTGGAACGACAACGAGCAGGGCCTGGACGAGCACATCGCCAGGATCAGCGCCGACATCGCCG
CAGGCGGAGTGATCGTGCAGGCCGTGCAGGACATCCTGCCCAGCCTGCACGCCCCCGCACCCGCCCCTAGGGGAG
GCGGGAGCGGCGAGCAGAAACTGATCAGCGAAGAGGACCTGGCCGCAAACGACATCCTGGACTACAAGGATGACG
ACGATAAGGTGTGACTCGAGCTAGTGACTGACTAGGATCTGGTTACCACTAAACCAGCCTCAAGAACACCCGAAT
GGAGTCTCTAAGCTACATAATACCAACTTACACTTACAAAATGTTGTCCCCCAAAATGTAGCCATTCGTATCTGC
TCCTAATAAAAAGAAAGTTTCTTCACATTCTAGAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAA
SEQ ID NO: 13 mutant avPAL complete mRNA sequence
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACCATGG
GCAAGACCCTGAGCCAGGCCCAGAGCAAGACCAGCAGCCAGCAGTTCAGCTTCACCGGCAACAGCAGCGCCAACG
TGATCATCGGCAACCAGAAGCTGACCATCAACGACGTGGCCAGGGTGGCCCGGAACGGCACCCTGGTGAGCCTGA
CCAACAACACCGACATCCTGCAGGGCATCCAGGCCAGCTGCGACTACATCAACAACGCCGTGGAGAGCGGCGAGC
CCATCTACGGCGTGACCAGCGGCTTCGGCGGAATGGCCAACGTGGCCATCAGCAGGGAGCAGGCCAGCGAGCTGC
AGACCAACCTGGTGTGGTTCCTGAAGACCGGAGCCGGCAACAAGCTGCCACTGGCCGACGTGAGAGCAGCCATGC
TCCTGAGGGCCAACAGCCACATGAGAGGCGCCAGCGGCATCAGGCTGGAGCTGATCAAGAGGATGGAGATCTTCC
TGAACGCCGGCGTGACCCCATACGTGTACGAGTTCGGCAGCATCGGCGCCAGCGGCGACCTGGTGCCCCTGAGCT
ACATCACCGGCAGCCTGATCGGCCTGGACCCCAGCTTCAAGGTGGACTTCAACGGCAAGGAGATGGACGCCCCAA
CCGCCCTGAGGCAGCTGAACCTGAGCCCCCTGACCCTGCTGCCCAAGGAGGGCCTGGCAATGATGAACGGCACCA
GCGTGATGACCGGCATCGCCGCCAACTGCGTGTACGACACCCAGATCCTGACCGCCATCGCAATGGGCGTGCACG
CCCTGGACATCCAGGCCCTGAACGGCACCAACCAGAGCTTCCACCCCTTCATCCACAACAGCAAGCCACACCCCG
GACAGCTGTGGGCCGCAGACCAGATGATCAGCCTGCTCGCCAACAGCCAGCTGGTGAGGGACGAGCTGGACGGCA
AGCACGACTACAGGGACCACGAGCTGATCCAGGACAGGTACAGCCTGAGGTGCCTGCCCCAGTACCTGGGCCCAA
TCGTGGACGGCATCAGCCAGATCGCCAAGCAGATCGAGATCGAGATCAACAGCGTGACCGACAACCCACTGATCG
ACGTGGACAACCAGGCCAGCTACCACGGCGGAAACTTCCTGGGCCAGTACGTGGGAATGGGCATGGACCACCTGA
GGTACTACATCGGCCTGCTCGCCAAGCACCTGGACGTGCAGATCGCCCTGCTCGCCAGCCCAGAGTTCAGCAACG
GACTGCCACCCAGCCTCCTGGGCAACAGGGAGCGGAAGGTGAACATGGGCCTGAAGGGACTGCAGATCTGCGGCA
ACAGCATCATGCCACTCCTGACCTTCTACGGCAACAGCATCGCCGACAGGTTCCCCACCCACGCCGAGCAGTTCA
ACCAGAACATCAACAGCCAGGGCTACACCAGCGCCACCCTGGCCAGGCGGAGCGTGGACATCTTCCAGAACTACG
TGGCCATCGCACTGATGTTCGGCGTGCAGGCCGTGGACCTGAGGACCTACAAGAAGACCGGCCACTACGACGCCA
GGGCCAGCCTGAGCCCCGCCACCGAGAGGCTGTACAGCGCCGTGAGGCACGTGGTCGGCCAGAAGCCCACCAGCG
ACAGGCCCTACATCTGGAACGACAACGAGCAGGGCCTGGACGAGCACATCGCCAGGATCAGCGCCGACATCGCCG
CAGGCGGAGTGATCGTGCAGGCCGTGCAGGACATCCTGCCCAGCCTGCACGCCCCCGCACCCGCCCCTAGGGGAG
GCGGGAGCGGCGAGCAGAAACTGATCAGCGAAGAGGACCTGGCCGCAAACGACATCCTGGACTACAAGGATGACG
ACGATAAGGTGTGACTCGAGCTAGTGACTGACTAGGATCTGGTTACCACTAAACCAGCCTCAAGAACACCCGAAT
GGAGTCTCTAAGCTACATAATACCAACTTACACTTACAAAATGTTGTCCCCCAAAATGTAGCCATTCGTATCTGC
TCCTAATAAAAAGAAAGTTTCTTCACATTCTAGAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAA
SEQ ID NO: 14 Arabidopsis thaliana PAL complete mRNA sequence
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACCATGG
ACCAGATTGAGGCCATGCTGTGCGGCGGCGGCGAGAAGACCAAGGTGGCCGTGACAACCAAGACCCTGGCCGACC
CTCTGAACTGGGGCCTGGCCGCCGACCAGATGAAGGGCAGCCACCTGGACGAGGTGAAGAAGATGGTGGAGGAGT
ACAGGAGGCCCGTGGTGAACCTGGGCGGCGAGACACTGACCATCGGCCAGGTGGCCGCCATCAGCACCGTGGGCG
GCAGCGTGAAGGTGGAGCTGGCCGAGACAAGCAGGGCCGGCGTGAAGGCCAGCAGCGACTGGGTGATGGAGAGCA
TGAACAAGGGCACCGACAGCTACGGCGTGACCACCGGCTTCGGCGCCACCAGCCACCGGAGGACCAAGAACGGCA
CCGCCCTGCAGACCGAGCTGATCAGGTTCCTGAACGCCGGCATCTTCGGCAACACCAAGGAGACATGCCACACCC
TGCCCCAGAGCGCCACCAGGGCCGCCATGCTGGTGAGGGTGAACACCCTGCTGCAGGGCTACAGCGGCATCAGGT
TCGAGATCCTGGAGGCCATCACCAGCCTGCTGAACCACAACATCAGCCCCAGCCTGCCCCTGAGGGGCACCATCA
CCGCCAGCGGCGACCTGGTGCCCCTGAGCTACATCGCCGGCCTGCTGACCGGCAGGCCCAACAGCAAGGCCACCG
GCCCCGACGGCGAGAGCCTGACCGCCAAGGAGGCCTTCGAGAAGGCCGGCATCAGCACCGGCTTCTTCGACCTGC
AGCCCAAGGAGGGCCTGGCCCTGGTGAACGGCACCGCCGTGGGCAGCGGCATGGCCAGCATGGTGCTGTTCGAGG
CCAACGTGCAGGCCGTGCTGGCCGAGGTGCTGAGCGCCATCTTCGCCGAGGTGATGAGCGGCAAGCCCGAGTTCA
CCGACCACCTGACCCACAGGCTGAAGCACCACCCCGGCCAGATCGAGGCCGCCGCCATCATGGAGCACATCCTGG
ACGGCAGCAGCTACATGAAGCTGGCCCAGAAGGTGCACGAGATGGACCCTCTGCAGAAGCCCAAGCAGGACAGGT
ACGCCCTGAGGACCAGCCCTCAGTGGCTGGGCCCTCAGATCGAGGTGATCAGGCAGGCCACCAAGAGCATCGAGA
GGGAGATCAACAGCGTGAACGACAATCCCCTGATCGACGTGAGCAGGAACAAGGCCATCCACGGCGGCAACTTCC
AGGGCACCCCTATCGGCGTGAGCATGGACAACACCAGGCTGGCCATCGCCGCCATCGGCAAGCTGATGTTCGCCC
AGTTCAGCGAGCTGGTGAACGACTTCTACAACAACGGCCTGCCCAGCAACCTGACCGCCAGCAGCAACCCCAGCC
TGGACTACGGCTTCAAGGGCGCCGAGATCGCTATGGCCAGCTACTGCAGCGAGCTGCAGTACCTGGCCAACCCCG
TGACCAGCCACGTGCAGAGCGCCGAGCAGCACAACCAGGACGTGAACAGCCTGGGCCTGATCAGCAGCAGGAAGA
CCAGCGAGGCCGTGGACATCCTGAAGCTGATGAGCACCACCTTCCTGGTGGGCATCTGCCAGGCCGTGGACCTGA
GGCACCTGGAGGAGAACCTGAGGCAGACCGTGAAGAACACCGTGAGCCAGGTGGCCAAGAAGGTGCTGACCACCG
GCATCAACGGCGAGCTGCACCCCAGCAGGTTCTGCGAGAAGGACCTGCTGAAGGTGGTGGACAGGGAGCAGGTGT
TCACCTACGTGGACGACCCCTGCAGCGCCACCTACCCTCTGATGCAGAGGCTGAGGCAGGTGATCGTGGACCACG
CCCTGAGCAACGGCGAGACAGAGAAGAACGCCGTGACCAGCATCTTCCAGAAGATCGGCGCCTTCGAGGAGGAGC
TGAAGGCCGTGCTGCCCAAGGAGGTGGAGGCCGCCAGGGCCGCCTACGGCAACGGCACCGCCCCTATCCCCAACC
GGATCAAGGAGTGCAGGAGCTACCCTCTGTACCGGTTCGTGAGGGAGGAGCTGGGCACCAAGCTGCTGACCGGCG
AGAAGGTGGTGAGCCCCGGCGAGGAGTTCGACAAGGTGTTCACCGCCATGTGCGAGGGCAAGCTGATCGACCCTC
TGATGGACTGCCTGAAGGAGTGGAACGGCGCCCCTATCCCCATCTGCCCTAGGGGAGGCGGGAGCGGCGAGCAGA
AACTGATCAGCGAAGAGGACCTGGCCGCAAACGACATCCTGGACTACAAGGACGACGACGACAAGTAGCTCGAGC
TAGTGACTGACTAGGATCTGGTTACCACTAAACCAGCCTCAAGAACACCCGAATGGAGTCTCTAAGCTACATAAT
ACCAACTTACACTTACAAAATGTTGTCCCCCAAAATGTAGCCATTCGTATCTGCTCCTAATAAAAAGAAAGTTTC
TTCACATTCTAGAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAA
SEQ ID NO: 15 Solanum lycopersicum PAL complete mRNA sequence
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACCATGG
CCTCTAGCATCGTGCAGAACGGCCACGTGAATGGCGAGGCTATGGACCTGTGCAAGAAGTCCATCAACGTGAACG
ACCCTCTGAACTGGGAGATGGCCGCCGAGAGCCTGAGGGGCAGCCACCTGGACGAGGTGAAGAAGATGGTGGACG
AGTTCAGGAAGCCCATCGTGAAGCTGGGCGGCGAGACACTGACCGTGGCCCAGGTGGCCAGCATCGCCAACGTGG
ACAACAAGAGCAACGGCGTGAAGGTGGAGCTGAGCGAGAGCGCCAGGGCCGGCGTGAAGGCCAGCAGCGACTGGG
TGATGGACAGCATGGGCAAGGGCACCGACAGCTACGGCGTGACCACCGGCTTCGGCGCCACCAGCCACAGGAGGA
CCAAGAACGGCGGCGCCCTGCAGAAGGAGCTGATCAGGTTCCTGAACGCCGGCGTGTTCGGCAACGGCACCGAGA
GCAGCCACACCCTGCCCCACAGCGCCACCAGGGCCGCCATGCTGGTGAGGATCAACACCCTGCTGCAGGGCTACA
GCGGCATCAGGTTCGAGATCCTGGAGGCCATCACCAAGCTGATCAACAGCAACATCACCCCTTGCCTGCCCCTGA
GGGGCACCATCACCGCCAGCGGCGACCTGGTGCCCCTGAGCTACATCGCCGGCCTGCTGACCGGCAGGCCCAACA
GCAAGGCCGTGGGCCCCAACGGCGAGAAGCTGAACGCCGAGGAGGCCTTCCACGTGGCCGGCGTGACCAGCGGCT
TCTTCGAGCTGCAGCCCAAGGAGGGCCTGGCCCTGGTGAACGGCACCGCCGTGGGCAGCGGCATGGCCAGCATGG
TGCTGTTCGAGAGCAACATCCTGGCCGTGATGAGCGAGGTGCTGAGCGCCATCTTCGCCGAGGTGATGAACGGCA
AGCCCGAGTTCACCGACTACCTGACCCACAAGCTGAAGCACCACCCCGGCCAGATCGAGGCCGCCGCCATCATGG
AGCACATCCTGGACGGCAGCAGCTACGTGAAGGAGGCCCAGAAGCTGCACGAGATGGACCCTCTGCAGAAGCCCA
AGCAGGACAGGTACGCCCTGAGGACCAGCCCTCAGTGGCTGGGCCCTCAGATCGAGGTGATCAGGGCCGCCACCA
AGATGATCGAGAGGGAGATCAACAGCGTGAACGACAATCCCCTGATCGACGTGAGCAGGAACAAGGCCCTGCACG
GCGGCAACTTCCAGGGCACCCCTATCGGCGTGAGCATGGACAACACCAGGCTGGCCCTGGCCAGCATCGGCAAGC
TGATGTTCGCCCAGTTCAGCGAGCTGGTGAACGACTACTACAACAACGGCCTGCCCAGCAACCTGACCGCCGGCA
GGAACCCCAGCCTGGACTACGGCTTCAAGGGCGCCGAGATCGCTATGGCCAGCTACTGCAGCGAGCTGCAGTTCC
TGGCCAACCCCGTGACCAACCACGTGCAGAGCGCCGAGCAGCACAACCAGGACGTGAACAGCCTGGGCCTGATCA
GCGCCAGGAAGACCGCCGAGGCCGTGGACATCCTGAAGCTGATGAGCAGCACCTACCTGGTGGCCCTGTGCCAGG
CCATCGACCTGAGGCACCTGGAGGAGAACCTGAAGAACGCCGTGAAGAACACCGTGAGCCAGGTGGCCAAGAAGA
CCCTGGCTATGGGCGCCAACGGCGAGCTGCACCCCGCCAGGTTCTGCGAGAAGGAGCTGCTGCAGGTGGTGGAGA
GGGAGTACCTGTTCACCTACGCCGACGACCCCTGCAGCAGCACCTACCCTCTGATGCAGAAGCTGAGGCAGGTGC
TGGTGGACCACGCCATGAAGAACGGCGAGAGCGAGAAGAACGTGAACAGCAGCATCTTCCAGAAGATCGTGGCCT
TCGAGGACGAGCTGAAGGCCGTGCTGCCCAAGGAGGTGGAGAGCGCCAGGGCCGTGGTGGAGAGCGGCAACCCCG
CCATCCCCAACAGGATCACCGAGTGCAGGAGCTACCCTCTGTACCGGCTGGTGAGGCAGGAGGTGGGCACCGAGC
TGCTGACCGGCGAGAAGGTGAGGAGCCCCGGCGAGGAGATCGACAAGGTGTTCACCGCCTTCTGCAACGGCCAGA
TCATCGACCCTCTGCTGGAGTGCCTGAAGTCCTGGAACGGCGCCCCTATCCCCATCTGCCCTAGGGGAGGCGGGA
GCGGCGAGCAGAAACTGATCAGCGAAGAGGACCTGGCCGCAAACGACATCCTGGACTACAAGGACGACGACGACA
AGTAGCTCGAGCTAGTGACTGACTAGGATCTGGTTACCACTAAACCAGCCTCAAGAACACCCGAATGGAGTCTCT
AAGCTACATAATACCAACTTACACTTACAAAATGTTGTCCCCCAAAATGTAGCCATTCGTATCTGCTCCTAATAA
AAAGAAAGTTTCTTCACATTCTAGAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAA
SEQ ID NO: 16 Nicotiana tabacum PAL complete mRNA sequence
AGGAAACTTAAGTCAACACAACATATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAGCCACCATGG
CCGGCGTGGCCCAGAACGGCCACCAGGAGATGGACTTCTGCGTTAAGGTGGACCCTCTGAACTGGGAGATGGCCG
CCGACAGCCTGAAGGGCAGCCACCTGGACGAGGTGAAGAAGATGGTGGCCGAGTTCAGGAAGCCCGTGGTGAAGC
TGGGCGGCGAGACACTGACCGTGGCCCAGGTGGCCGCCATCGCCGCCAAGGACAACGCCAAGACCGTGAAGGTGG
AGCTGAGCGAGGGCGCCAGGGCCGGCGTGAAGGCCAGCAGCGACTGGGTGATGGACAGCATGAGCAAGGGCACCG
ACAGCTACGGCGTGACCACCGGCTTCGGCGCCACCAGCCACAGGAGGACCAAGAACGGCGGCGCCCTGCAGAAGG
AGCTGATCAGGTTCCTGAACGCCGGCGTGTTCGGCAACGGCACCGAGAGCTGCCACACCCTGCCCCAGAGCGGCA
CCAGGGCCGCCATGCTGGTGAGGATCAACACCCTGCTGCAGGGCTACAGCGGCATCAGGTTCGAGATCCTGGAGG
CCATCACCAAGCTGCTGAACCACAACGTGACCCCTTGCCTGCCCCTGAGGGGCACCATCACCGCCAGCGGCGACC
TGGTGCCCCTGAGCTACATCGCCGGCCTGCTGACCGGCCGGCCCAACAGCAAGGCCATCGGCCCCAACGGCGAGA
CACTGAACGCCGAGGAGGCCTTCAGGGTGGCCGGCGTGAACAGCGGCTTCTTCGAGCTGCAGCCCAAGGAGGGCC
TGGCCCTGGTGAACGGCACCGCCGTGGGCAGCGGCCTGGCCAGCATGGTGCTGTTCGACGCCAACATCCTGGCCG
TGTTCAGCGAGGTGCTGAGCGCCATCTTCGCCGAGGTGATGAACGGCAAGCCCGAGTTCACCGACCACCTGACCC
ACAAGCTGAAGCACCACCCCGGCCAGATCGAGGCCGCCGCCATCATGGAGCACATCCTGGACGGCAGCAGCTACG
TGAAGGCCCCTCAGAAGCTGCACGAGACAGACCCTCTGCAGAAGCCCAAGCAGGACAGGTACGCCCTGAGGACCA
GCCCTCAGTGGCTGGGCCCTCAGATCGAGGTGATCAGGAGCGCCACCAAGATGATCGAGAGGGAGATCAACAGCG
TGAACGACAATCCCCTGATCGACGTGAGCAGGAACAAGGCCCTGCACGGCGGCAACTTCCAGGGCACCCCTATCG
GCGTGAGCATGGACAACGCCAGGCTGGCCCTGGCCAGCATCGGCAAGCTGATGTTCGCCCAGTTCAGCGAGCTGG
TGAACGACTACTACAACAACGGCCTGCCCAGCAACCTGACCGCCGGCAGGAACCCCAGCCTGGACTACGGCTTCA
AGGGCAGCGAGATCGCTATGGCCAGCTACTGCAGCGAGCTGCAGTTCCTGGCCAACCCCGTGACCAACCACGTGC
AGAGCGCCGAGCAGCACAACCAGGACGTGAACAGCCTGGGCCTGATCAGCGCCAGGAAGACCGCCGAGGCCGTGG
ACATCCTGAAGCTGATGAGCAGCACCTACCTGGTGGCCCTGTGCCAGGCCATCGACCTGAGGCACCTGGAGGAGA
ACCTGAGGAACGCCGTGAAGAACACCGTGAGCCAGGTGGCCAAGAGGACCCTGACAATGGGCGCCAACGGCGAGC
TGCACCCCAGCAGGTTCTGCGAGAAGGACCTGCTGAGGGTGGTGGACAGGGAGTACGTGTTCAGGTACGCCGACG
ACGCCTGCAGCGCCAACTACCCTCTGATGCAGAAGCTGAGGCAGGTGCTGGTGGACCACGCCCTGGAGAACGGCG
AGAACGAGAAGAACGCCAACAGCAGCATCTTCCAGAAGATCCTGGCCTTCGAGGGCGAGCTGAAGGCCGTGCTGC
CCAAGGAGGTGGAGAGCGCCAGGATCAGCCTGGAGAACGGCAACCCCGCCATCGCCAACAGGATCAAGGAGTGCA
GGAGCTACCCTCTGTACCGGTTCGTGAGGGAGGAGCTGGGCGCCGAGCTGCTGACCGGCGAGAAGGTGAGGAGCC
CCGGCGAGGAGTGCGACAAGGTGTTCACCGCCATGTGCAACGGCCAGATCATCGACAGCCTGCTGGAGTGCCTGA
AGGAGTGGAACGGCGCCCCTCTGCCCATCTGCCCTAGGGGAGGCGGGAGCGGCGAGCAGAAACTGATCAGCGAAG
AGGACCTGGCCGCAAACGACATCCTGGACTACAAGGACGACGACGACAAGTAGCTCGAGCTAGTGACTGACTAGG
ATCTGGTTACCACTAAACCAGCCTCAAGAACACCCGAATGGAGTCTCTAAGCTACATAATACCAACTTACACTTA
CAAAATGTTGTCCCCCAAAATGTAGCCATTCGTATCTGCTCCTAATAAAAAGAAAGTTTCTTCACATTCTAGAAA
AAAAAAAAAAAAAAAAAAAAAAAAAAAAAAAAAAAAAAAAAAAAAAAAAAAAAAAAAAAAAAAAAAAAAAAAAAA
AAAAAAAAAAAAAAAAAAA
SEQ ID NO: 17 avPAL protein (Q3M5Z3)

| | |
|---|---|
| 1 | MKTLSQAQSK TSSQQFSFTG NSSANVIIGN QKLTINDVAR VARNGTLVSL TNNTDILQGI |
| 61 | QASCDYINNA VESGEPIYGV TSGFGGMANV AISREQASEL QTNLVWFLKT GAGNKLPLAD |
| 121 | VRAAMLLRAN SHMRGASGIR LELIKRMEIF LNAGVTPYVY EFGSIGASGD LVPLSYITGS |
| 181 | LIGLDPSFKV DFNGKEMDAP TALRQLNLSP LTLLPKEGLA MMNGTSVMTG IAANCVYDTQ |
| 241 | ILTAIAMGVH ALDIQALNGT NQSFHPFIHN SKPHPGQLWA ADQMISLLAN SQLVRDELDG |
| 301 | KHDYRDHELI QDRYSLRCLP QYLGPIVDGI SQIAKQIEIE INSVTDNPLI DVDNQASYHG |
| 361 | GNFLGQYVGM GMDHLRYYIG LLAKHLDVQI ALLASPEFSN GLPPSLLGNR ERKVNMGLKG |
| 421 | LQICGNSIMP LLTFYGNSIA DRFPTHAEQF NQNINSQGYT SATLARRSVD IFQNYVAIAL |
| 481 | MFGVQAVDLR TYKKTGHYDA RACLSPATER LYSAVRHVVG QKPTSDRPYI WNDNEQGLDE |
| 541 | HIARISADIA AGGVIVQAVQ DILPCLH |

SEQ ID NO: 18 Arabidopsis thaliana PAL protein (P35510)

| | |
|---|---|
| 1 | MEINGAHKSN GGGVDAMLCG GDIKTKNMVI NAEDPLNWGA AAEQMKGSHL DEVKRMVAEF |
| 61 | RKPVVNLGGE TLTIGQVAAI STIGNSVKVE LSETARAGVN ASSDWVMESM NKGTDSYGVT |
| 121 | TGFGATSHRR TKNGVALQKE LIRFLNAGIF GSTKETSHTL PHSATRAAML VRINTLLQGF |
| 181 | SGIRFEILEA ITSFLNNNIT PSLPLRGTIT ASGDLVPLSY IAGLLTGRPN SKATGPNGEA |
| 241 | LTAEEAFKLA GISSGFFDLQ PKEGLALVNG TAVGSGMASM VLFETNVLSV LAEILSAVFA |
| 301 | EVMSGKPEFT DHLTHRLKHH PGQIEAAAIM EHILDGSSYM KLAQKLHEMD PLQKPKQDRY |
| 421 | TRLAIAAIGK LMFAQFSELV NDFYNNGLPS NLTASRNPSL DYGFKGAEIA MASYCSELQY |
| 481 | LANPVTSHVQ SAEQHNQDVN SLGLISSRKT SEAVDILKLM STTELVAICQ AVDLRHLEEN |
| 541 | LRQTVKNTVS QVAKKVLTTG VNGELHPSRF CEKDLLKVVD REQVYTYADD PCSATYPLIQ |
| 601 | KLRQVIVDHA LINGESEKNA VTSIFHKIGA FEEELKAVLP KEVEAARAAY DNGTSAIPNR |
| 661 | IKECRSYPLY RFVREELGTE LLTGEKVTSP GEEFDKVFTA ICEGKIIDPM MECLNEWNGA |
| 721 | PIPIC |

SEQ ID NO: 19 Solanum lycopersicum PAL protein (P35511)

| | |
|---|---|
| 1 | MDLCKKSIND PLNWEMAADS LRGSHLDEVK KMVDEFRKPI VKLGGETLSV AQVASIANVD |
| 61 | DKSNGVKVEL SESARAGVKA SSDWVMDSMS KGTDSYGVTA GFGATSHRRT KNGGALQKEL |
| 121 | IRFLNAGVFG NGIESFHTLP HSATRAAMLV RINTLLQGYS GIRFEILEAI TKLINSNITP |
| 181 | CLPLRGTITA SGDLVPLSYI AGLLTGRPNS KAVGPNGEKL NAEEAFCVAG ISGGFFELQP |
| 241 | KEGLALVNGT AVGSAMASIV LFESNIFAVM SEVLSAIFTE VMNGKPEFTD YLTHKLKHHP |
| 301 | GQIEAAAIME HILDGSSYVK VAQKLHEMDP LQKPKQDRYA LRTSPQWLGP QIEVIRAATK |
| 361 | MIEREINSVN DNPLIDVSRN KALHGGNFQG TPIGVSMDNT RLALASIGKL MFAQFSELVN |
| 421 | DYYNNGLPSN LTAGRNPSLD YGFKGAEIAM ASYCSELQFL ANPVTNHVQS AEQHNQDVNS |
| 481 | LGLISARKTA KAVDILKIMS STYLVALCQA IDLRHLEENL KSVVKNTVSQ VAKRTLTMGA |
| 541 | NGELHPARFS EKELLRVVDR EYLFAYADDP CSSNYPLMQK LRQVLVDQAM KNGESEKNVN |
| 601 | SSIFQKIGAF EDELIAVLPK EVESVRAVFE SGNPLIRNRI TECRSYPLYR LVREELGTEL |
| 661 | LTGEKVRSPG EEIDKVFTAI CNGQIIDPLL ECLKSWNGAP LPIC |

SEQ ID NO: 20 Nicotiana tabacum PAL protein (P25872)

| | |
|---|---|
| 1 | MASNGHVNGG ENFELCKKSA DPLNWEMAAE SLRGSHLDEV KKMVSEFRKP MVKLGGESLT |
| 61 | VAQVAAIAVR DKSANGVKVE LSEEARAGVK ASSDWVMDSM NKGTDSYGVT TGFGATSHRR |
| 121 | TKNGGALQKE LIRFLNAGVF GNGTETSHTL PHSATRAAML VRINTLLQGY SGIRFEILEA |
| 181 | ITKLINSNIT PCLPLRGTIT ASGDLVPLSY IAGLLTGRPN SKAVGPNGET LNAEEAFRVA |
| 241 | GVNGGFFELQ PKEGLALVNG TAVGSGMASM VLFDSNILAV MSEVLSAIFA EVMNGKPEFT |
| 301 | DHLTHKLKHH PGQIEAAAIM EHILDGSSYV KAAQKLHEMD PLQKPKQDRY ALRTSPQWLG |
| 361 | PQIEVIRAAT KMIEREINSV NDNPLIDVSR NKALHGGNFQ GTPIGVSMDN ARLALASIGK |
| 421 | LMFAQFSELV NDYYNNGLPS NLTASRNPSL DYGFKGAEIA MASYCSELQF LANPVTNHVQ |
| 481 | SAEQHNQDVN SLGLISARKT AEAVDILKLM SSTYLVALCQ AIDLRHLEEN LKNAVKNTVS |
| 541 | QVAKRTLTMG ANGELHPARF CEKELLRIVD REYLFAYADD PCSCNYPLMQ KLRQVLVDHA |
| 601 | MNNGESEKNV NSSIFQKIGA FEDELKAVLP KEVESARAAL ESGNPAIPNR ITECRSYPLY |
| 661 | RFVRKELGTE LLTGEKVRSP GEECDKVFTA MCNGQIIDPM LECLKSWNGA PLPIC |

SEQ ID NO: 21 Kozak Sequence
GCCACC
SEQ ID NO: 22 Partial Kozak Sequence
GCCA
SEQ ID NO: 23 Triple Stop Codon
AUAAGUGAA
SEQ ID NO: 24 TEV (5′ UTR)
UCAACACAACAUAUACAAAACAAACGAAUCUCAAGCAAUCAAGCAUUCUACUUCUAUUGCAGCAAUUUAAAUCAU
UUCUUUUAAAGCAAAAGCAAUUUUCUGAAAAUUUUCACCAUUUACGAACGAUAG
SEQ ID NO: 25 AT1G58420 (5′ UTR)
AUUAUUACAUCAAAACAAAAAGCCGCCA
SEQ ID NO: 26 HUMAN ALBUMIN (5′ UTR)
AAUUAUUGGUUAAAGAAGUAUAUUAGUGCUAAUUUCCCUCCGUUUGUCCUAGCUUUUCUCUUCUGUCAACCCCAC
ACGCCUUUGGCACA
SEQ ID NO: 27 SYNECHOCYSTIS sp. PCC6803 POTASSIUM CHANNEL (SynK) (5′ UTR)
AACUUAAAAAAAAAAAUCAAA
SEQ ID NO: 28 MOUSE BETA GLOBIN (5′ UTR)
CACAUUUGCUUCUGACAUAGUUGUGUUGACUCACAACCCCAGAAACAGACAUC
SEQ ID NO: 29 HUMAN BETA GLOBIN (5′ UTR)
ACAUUUGCUUCUGACACAACUGUGUUCACUAGCAACCUCAAACAGACACC
SEQ ID NO: 30 MOUSE ALBUMIN (5′ UTR)
UGCACACAGAUCACCUUUCCUAUCAACCCCACUAGCCUCUGGCAAA
SEQ ID NO: 31 HUMAN HAPTOGLOBIN (5′ UTR)
AUAAAAAGACCAGCAGAUGCCCCACAGCACUGCUCUUCCAGAGGCAAGACCAACCAAG
SEQ ID NO: 32 HUMAN TRANSTHYRETIN (5′ UTR)
AGACAAGGUUCAUAUUUGUAUGGGUUACUUAUUCUCUCUUUGUUGACUAAGUCAAUAAUCAGAAUCAGCAGGUUU
GCAGUCAGAUUGGCAGGGAUAAGCAGCCUAGCUCAGGAGAAGUGAGUAUAAAAGCCCCAGGCUGGGAGCAGCCAU
CACAGAAGUCCACUCAUUCUUGGCAGG
SEQ ID NO: 33 HUMAN COMPLEMENT C3 (5′ UTR)
AGAUAAAAAGCCAGCUCCAGCAGGCGCUGCUCACUCCUCCCCAUCCUCUCCCUCUGUCCCUCUGUCCCUCUGACC
CUGCACUGUCCCAGCACC
SEQ ID NO: 34 HUMAN COMPLEMENT C5 (5′ UTR)
UAUAUCCGUGGUUUCCUGCUACCUCCAACC
SEQ ID NO: 35 HUMAN ALPHA-1-ANTITRYPSIN (5′ UTR)
GGCACCACCACUGACCUGGGACAGUGAAUCGACA
SEQ ID NO: 36 HUMAN ALPHA-1-ANTICHYMOTRYPSIN (5′ UTR)
AUUCAUGAAAAUCCACUACUCCAGACAGACGGCUUUGGAAUCCACCAGCUACAUCCAGCUCCCUGAGGCAGAGUU
GAGA
SEQ ID NO: 37 HUMAN INTERLEUKIN 6 (5′ UTR)
AAUAUUAGAGUCUCAACCCCCAAUAAAUAUAGGACUGGAGAUGUCUGAGGCUCAUUCUGCCCUCGAGCCCACCGG
GAACGAAAGAGAAGCUCUAUCUCCCCUCCAGGAGCCCAGCU
SEQ ID NO: 38 HUMAN FIBRINOGEN ALPHA CHAIN (5′ UTR)
AGGAUGGGAACUAGGAGUGGCAGCAAUCCUUUCUUUCAGCUGGAGUGCUCCUCAGGAGCCAGCCCCACCCUUAGA
AAAG
SEQ ID NO: 39 HUMAN APOLIPOPROTEIN E (5′ UTR)
AGGGGGAGCCCUAUAAUUGGACAAGUCUGGGAUCCUUGAGUCCUACUCAGCCCCAGCGGAGGUGAAGGACGUCCU
UCCCCAGGAGCCGACUGGCCAAUCACAGGCAGGAAG
SEQ ID NO: 40 ALANINE AMINOTRANSFERASE 1 (5′ UTR)
AGACGGGUGGGGCGGGGCCCAACUGUCCCCAGCUCCUUCAGCCCUUUCUGUCCCUCCCAGUGAGGCCAGCUGCGG
UGAAGAGGGUGCUCUCUUGCCUGGAGUUCCCUCUGCUACGGCUGCCCCCUCCCAGCCCUGGCCCACUAAGCCAGA
CCCAGCUGUCGCCAUUCCCACUUCUGGUCCUGCCACCUCCUGAGCUGCCUUCCCGCCUGGUCUGGGUAGAGUC
SEQ ID NO: 41 HUMAN ANTITHROMBIN (5′ UTR)
UCUGCCCCACCCUGUCCUCUGGAACCUCUGCGAGAUUUAGAGGAAAGAACCAGUUUUCAGGCGGAUUGCCUC
AGAUCACACUAUCUCCACUUGCCCAGCCCUGUGGAAGAUUAGCGGCC
SEQ ID NO: 42 XBG (3′ UTR)
CUAGUGACUGACUAGGAUCUGGUUACCACUAAACCAGCCUCAAGAACACCCGAAUGGAGUCUCUAAGCUACAUAA
UACCAACUUACACUUACAAAAUGUUGUCCCCCAAAAUGUAGCCAUUCGUAUCUGCUCCUAAUAAAAAGAAAGUUU
CUUCACAU
SEQ ID NO: 43 HUMAN HAPTOGLOBIN (3′ UTR)
UGCAAGGCUGGCCGGAAGCCCUUGCCUGAAAGCAAGAUUUCAGCCUGGAAGAGGGCAAAGUGGACGGGAGUGGAC
AGGAGUGGAUGCGAUAAGAUGUGGUUUGAAGCUGAUGGGUGCCAGCCCUGCAUUGCUGAGUCAAUCAAUAAAGAG
CUUUCUUUUGACCCAU
SEQ ID NO: 44 HUMAN APOLIPOPROTEIN E (3′ UTR)
ACGCCGAAGCCUGCAGCCAUGCGACCCCACGCCACCCCGUGCCUCCUGCCUCCGCGCAGCCUGCAGCGGGAGACC
CUGUCCCCGCCCCAGCCGUCCUCCUGGGGUGGACCCUAGUUUAAUAAAGAUUCACCAAGUUUCACGCA
SEQ ID NO: 45 MOUSE ALBUMIN (3′ UTR)
ACACAUCACAACCACAACCUUCUCAGGCUACCCUGAGAAAAAAAGACAUGAAGACUCAGGACUCAUCUUUUCUGU
UGGUGUAAAAUCAACACCCUAAGGAACACAAAUUUCUUUAAACAUUUGACUUCUUGUCUCUGUGCUGCAAUUAAU
AAAAAAUGGAAAGAAUCUAC
SEQ ID NO: 46 HUMAN ALPHA GLOBIN (3′ UTR)
GCUGGAGCCUCGGUAGCCGUUCCUCCUGCCCGCUGGGCCUCCCAACGGGCCCUCCUCCCCUCCUUGCACCGGCCC
UUCCUGGUCUUUGAAUAAAGUCUGAGUGGGCAGCA
SEQ ID NO: 47 MOUSE BETA GLOBIN (3′ UTR)
ACCCCCUUUCCUGCUCUUGCCUGUGAACAAUGGUUAAUUGUUCCCAAGAGAGCAUCUGUCAGUUGUUGGCAAAAU
GAUAAAGACAUUUGAAAAUCUGUCUUCUGACAAAUAAAAAGCAUUUAUUUCACUGCAAUGAUGUUUU
SEQ ID NO: 48 HUMAN BETA GLOBIN (3′ UTR)
GCUCGCUUUCUUGCUGUCCAAUUUCUAUUAAAGGUUCCUUUGUUCCCUAAGUCCAACUACUAAACUGGGGGAUAU
UAUGAAGGGCCUUGAGCAUCUGGAUUCUGCCUAAUAAAAAACAUUUAUUUUCAUUGCAA
SEQ ID NO: 49 HUMAN GROWTH FACTOR (3′ UTR)
UGGCAUCCCUGUGACCCCUCCCCAGUGCCUCUCCUGGCCCUGGAAGUUGCCACUCCAGUGCCCACCAGCCUUGUC
CUAAUAAAAUUAAGUUGCAUCAUUUUGUCUG
SEQ ID NO: 50 HUMAN ANTITHROMBIN (3′ UTR)
AAUGUUCUUAUUCUUUGCACCUCUUCCUAUUUUUGGUUUGUGAACAGAAGUAAAAAUAAAUACAAACUACUUCCA
UCUCA
SEQ ID NO: 51 HUMAN COMPLEMENT C3 (3′ UTR)
CCACACCCCCAUUCCCCCACUCCAGAUAAAGCUUCAGUUAUAUCUCACGUGUCUGGAGUUCUUUGCCAAGAGGGA
GAGGCUGAAAUCCCCAGCCGCCUCACCUGCAGCUCAGCUCCAUCCUACUUGAAACCUCACCUGUUCCCACCGCAU
UUUCUCCUGGCGUUCGCCUGCUAGUGUG
SEQ ID NO: 52 HUMAN HEPCIDIN (3′ UTR)
AACCUACCUGCCCUGCCCCCGUCCCCUCCCUUCCUUAUUUAUUCCUGCUGCCCCAGAACAUAGGUCUUGGAAUAA
AAUGGCUGGUUCUUUUGUUUUCCAAA
SEQ ID NO: 53 HUMAN FIBRINOGEN ALPHA CHAIN (3′ UTR)
ACUAAGUUAAAUAUUUCUGCACAGUGUUCCCAUGGCCCCUUGCAUUUCCUUCUUAACUCUCUGUUACACGUCAUU
GAAACUACACUUUUUUGGUCUGUUUUUGUGCUAGACUGUAAGUUCCUUGGGGGCAGGGCCUUUGUCUGUCUCAUC
UCUGUAUUCCCAAAUGCCUAACAGUACAGAGCCAUGACUCAAUAAAUACAUGUUAAAUGGAUGAAUGAAUUCCUC
UGAAACUCU
SEQ ID NO: 54 ALANINE AMINOTRANSFERASE 1 (3′ UTR)
GCACCCCAGCUGGGGCCAGGCUGGGUCGCCCUGGACUGUGUGCUCAGGAGCCCUGGGAGGCUCUGGAGCCCACUG
UACUUGCUCUUGAUGCCUGGCGGGGUGGGGUGGGGGGGGUGCUGGGCCCCUGCCUCUCUGCAGGUCCCUAAUAAA
GCUGUGUGGCAGUCUGACUCC
SEQ ID NO: 55 MOUSE MALAT-1 (3′ UTR)
GAUUCGUCAGUAGGGUUGUAAAGGUUUUUCUUUUCCUGAGAAAACAACCUUUUGUUUUCUCAGGUUUUGCUUUUU
GGCCUUUCCCUAGCUUUAAAAAAAAAAAAGCAAAA
SEQ ID NO: 56 ALANINE AMINOTRANSFERASE (3′ UTR)
GGACGC CUCAGGCACC GGAGCCAGAC CCUCCCAAGA CCACCCAGGC CUUCCUCAAG GACUCUGCCU
CAGACCUCAG ACAGGCCACC AACGCUGUUC AUCUUCAUUU CCCCAAGGAG ACUUCUUUCU
UUGUGCCUUG AUGUUUGAGA GUUCUUCGAG CAAACAGUGG UUUUGCAAUG UCUCACAGGC
CCUGUUUUUG UUUUUGUUUU UGUUUUGUUU UGUUUUGUUC UUUUUUUAAA UGCAACCAAA
GUAGAGUCAA CCUGCUCGGC AGAUGUACUU GGAUUCUCUG AAUCGCUAUU CUGUUUGGAG
AGUUCCUUUG GGUCUUAAGC AGCCAGAGUA CAUGGAAAUG AGAUUAUGUC AGAUCUGGAG
AAACAAGCAG GUGUUGGGAA AUAUGUGACU UGACAUGAUA AGGGCUGGGA AUCCAGAAAU
CAAUAGUGAG AUCCAUGAAA UCAAACCCUG ACCAGUGUGA AAAUGUAGCC UUUUGGACAG
UAAGCCUGCA AGUCUAGUGA GAACUCAGAG AAAGCUGACC AUUCUGGUCU GAAGAUAGGC
AGCGCAUCAC AGGCAAGAAU AUCGAAGUCA GUAGUAGGAC AGGGGUCACA UCAGAUACCA
GCUCAAAUUG CACUAGCUAU CUAGAACAGU UUUCUCCAGG UUUGCCUGAG CCUUGAUGCA
UACCAUCGCC CUCUGCUGGU CGCAGCAGAG AUAAGCAAGG GCUGAAAAUG GAGGCAAUCC
UUUCCCAAGG CCCUGAAAGU UGUUUUUCAU GGUUUCAAAC UGAAUUUGGC UCAUUUGUAA
CUAACUGAUC ACGGUGCCUG GUUACACUGG CUGCCAAGAA GGAGCGCAUG CAAUCUGAUU
CAGUGCUCUC UUCACAUCAG UUUCCUGCCU CCCUCCCUCA UCUGCGGACA GCAUCCUAUC
UCAUCAGGCU UCCCUGUGUG UCACAAAGUA GCAGCCACCA AGCAAAUAUA UUCCUUGAAU
UAGCACACCU GGGUGGGCCA UGUGCGCACC AAGGAAACAG GUGCUAUAGG GAGCGCCAGG
CCAGGCUUGU CUCUUAACUG UCUCGUUCUU CAGUGAGAGU GGGAAAGCUG UCCGGAGCUC
CCGCGCAGGA GCCUGGGUAC CCACGCAGCG AGUCAAGGGA GUUUUCGGAG CCAGAGAGAG
AAAGAUGUGA AGGCUGUGGA GUAAGGCUGA AACCAGCCUC CUGCCCUAUA GUCCCACACU
GCAGGGGGUG CGACUUUAAA ACAGAACUUC AAGUUGUUAA CACUCACAAG CAUUGCAUUA
CUGUGAAGGA AGUAGCCGCA UCCAUAACAG GAUGUGAUGG UCUACAGCUU UUCCUUUAAA
AGCUGAAAAG GUACCAUGUG UGCUCGCUAG GCAUAUAAUC CAGAUAUGCU CCAGAGUUCU
GAGAUUCUUC CAUGAAAGGU UAACUAGAAG CUAGAAUAUU UUUUUAUAUU UUUGUAACAA
UUGGCUUUUU UCAUGGGGGG AGGGGAGUAG AGGGUUAGUA UUUAUAGUCC UAACAAGUCC
AAAAAUUUUU AUAAGUGUCU UCAGAUUAUA AAUAACCCUC CAAAUUUUGC AAUGUUUACA
UGUUUUUUUU UUAAGAUGAC AAAUAUGCUU GAUUUGCUUU UUAAAUAAAA GUUUAGCUGU
UCUAAGAGAU UAACUUCAAG UAGGAUGGCU GGUUAUGAUA GUUUGGAUUU UCUACAGGUU
CUGUUGCCAU GCCUUUUGGG UUUCAGCAUC ACUCGAGUCG CAGCAUGUGG GUGGGGCUGU
GGAAACCUGG CCAGGCUGGA CCUGGUCAGC CACACCUCAG AGACAUUGUU UCCAUUUGGA
UGUGAGCAGG CGCAGGCCUG CAUGCUCUUU CCUACUUAGC AUCAUCAGUU CUUCCGCCUC
CUUAGCAUGG UUCUUUGUAA CAGCCAUGCU GGGAAGCUCU GAACAAUAAA AUACUUCCAG AGUGGU

TABLE 3
Description of sequences

| SEQ ID NO | Description |
|---|---|
| SEQ ID NO: 1 | Anabaena variabilis (Trichormus variabilis) PAL ORF (with Myc |
| | and FLAG tags) |
| SEQ ID NO: 2 | Anabaena variabilis (Trichormus variabilis) PAL ORF |
| SEQ ID NO: 3 | Anabaena variabilis (Trichormus variabilis) mutant PAL ORF (with |
| | Myc and FLAG tags) |
| SEQ ID NO: 4 | Anabaena variabilis (Trichormus variabilis) mutant PAL ORF |
| SEQ ID NO: 5 | Arabidopsis thaliana PAL ORF |
| SEQ ID NO: 6 | Solanum lycopersicum PAL ORF |
| SEQ ID NO: 7 | Nicotiana tabacum PAL ORF |
| SEQ ID NO: 8 | TEV 5′ UTR |
| SEQ ID NO: 9 | XBG 3′ UTR |
| SEQ ID NO: 10 | avPAL complete mRNA sequence (with FLAG and Myc tags) |
| SEQ ID NO: 11 | avPAL complete mRNA sequence |
| SEQ ID NO: 12 | mutant avPAL complete mRNA sequence (with FLAG and Myc |
| | tags) |
| SEQ ID NO: 13 | mutant avPAL complete mRNA sequence |
| SEQ ID NO: 14 | Arabidopsis thaliana PAL complete mRNA sequence |
| SEQ ID NO: 15 | Solanum lycopersicum PAL complete mRNA sequence |
| SEQ ID NO: 16 | Nicotiana tabacum PAL complete mRNA sequence |
| SEQ ID NO: 17 | Anabaena variabilis (Trichormus variabilis) PAL protein |
| | (Q3M5Z3) |
| SEQ ID NO: 18 | Arabidopsis thaliana PAL protein (P35510) |
| SEQ ID NO: 19 | Solanum lycopersicum PAL protein (P35511) |
| SEQ ID NO: 20 | Nicotiana tabacum PAL protein (P25872) |
| SEQ ID NO: 21 | Kozak sequence |
| SEQ ID NO: 22 | Partial Kozak sequence |
| SEQ ID NO: 23 | Triple Stop Codon |
| SEQ ID NO: 24 | TEV (5′ UTR) |
| SEQ ID NO: 25 | AT1G58420 (5′ UTR) |
| SEQ ID NO: 26 | HUMAN ALBUMIN (5′ UTR) |
| SEQ ID NO: 27 | SYNECHOCYSTIS sp. PCC6803 POTASSIUM CHANNEL (SynK) |
| | (5′ UTR) |
| SEQ ID NO: 28 | MOUSE BETA GLOBIN (5′ UTR) |
| SEQ ID NO: 29 | HUMAN BETA GLOBIN (5′ UTR) |
| SEQ ID NO: 30 | MOUSE ALBUMIN (5′ UTR) |
| SEQ ID NO: 31 | HUMAN HAPTOGLOBIN (5′ UTR) |
| SEQ ID NO: 32 | HUMAN TRANSTHYRETIN (5′ UTR) |
| SEQ ID NO: 33 | HUMAN COMPLEMENT C3 (5′ UTR) |
| SEQ ID NO: 34 | HUMAN COMPLEMENT C5 (5′ UTR) |
| SEQ ID NO: 35 | HUMAN ALPHA-1-ANTITRYPSIN (5′ UTR) |
| SEQ ID NO: 36 | HUMAN ALPHA-1-ANTICHYMOTRYPSIN (5′ UTR) |
| SEQ ID NO: 37 | HUMAN INTERLEUKIN 6 (5′ UTR) |
| SEQ ID NO: 38 | HUMAN FIBRINOGEN ALPHA CHAIN (5′ UTR) |
| SEQ ID NO: 39 | HUMAN APOLIPOPROTEIN E (5′ UTR) |
| SEQ ID NO: 40 | ALANINE AMINOTRANSFERASE 1 (5′ UTR) |
| SEQ ID NO: 41 | HUMAN ANTITHROMBIN (5′ UTR) |
| SEQ ID NO: 42 | XBG (3′ UTR) |
| SEQ ID NO: 43 | HUMAN HAPTOGLOBIN (3′ UTR) |
| SEQ ID NO: 44 | HUMAN APOLIPOPROTEIN E (3′ UTR) |
| SEQ ID NO: 45 | MOUSE ALBUMIN (3′ UTR) |
| SEQ ID NO: 46 | HUMAN ALPHA GLOBIN (3′ UTR) |
| SEQ ID NO: 47 | MOUSE BETA GLOBIN (3′ UTR) |
| SEQ ID NO: 48 | HUMAN BETA GLOBIN (3′ UTR) |
| SEQ ID NO: 49 | HUMAN GROWTH FACTOR (3′ UTR) |
| SEQ ID NO: 50 | HUMAN ANTITHROMBIN (3′ UTR) |
| SEQ ID NO: 51 | HUMAN COMPLEMENT C3 (3′ UTR) |
| SEQ ID NO: 52 | HUMAN HEPCIDIN (3′ UTR) |
| SEQ ID NO: 53 | HUMAN FIBRINOGEN ALPHA CHAIN (3′ UTR) |
| SEQ ID NO: 54 | ALANINE AMINOTRANSFERASE 1 (3′ UTR) |
| SEQ ID NO: 55 | MOUSE MALAT-1 (3′ UTR) |
| SEQ ID NO: 56 | ALANINE AMINOTRANSFERASE (3′ UTR) |

As used herein, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of +20%, or ±10%, or 5%, or even ±1% from the specified value, as such variations are appropriate for the disclosed methods or to perform the disclosed methods.

Ranges: throughout this disclosure, various aspects can be presented in range format. It should be understood that any description in range format is merely for convenience and brevity and not meant to be limiting. Accordingly, the description of a range should be considered to have specifically disclosed all possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example 1, 2, 2.1, 2.2, 2.5, 3, 4, 4.75, 4.8, 4.85, 4.95, 5, 5.5, 5.75, 5.9, 5.00, and 6. This applies to a range of any breadth.

Unless defined otherwise, all technical and scientific terms used herein have the same meaning as is commonly understood by one of skill in the art to which this invention belongs.

Any and all references and citations to other documents, such as patents, patent applications, patent publications, journals, books, papers, web contents that have been made throughout this disclosure are hereby incorporated herein in their entirety for all purposes.

Although the invention has been described with reference to the above examples, it will be understood that modifications and variations are encompassed within the spirit and scope of the invention. Accordingly, the invention is limited only by the following claims.

What is claimed is:

1. A polynucleotide for expressing bacterial phenylalanine ammonia lyase (PAL) or a fragment thereof, wherein the polynucleotide comprises natural and chemically modified nucleotides, and wherein the polynucleotide is expressible to provide bacterial PAL or a fragment thereof having PAL activity.

2. The polynucleotide of claim 1, wherein the bacterial PAL is an Anabaena PAL.

3. The polynucleotide of claim 1 or claim 2, wherein the bacterial PAL is an Anabaena variabilis PAL.

4. The polynucleotide of any one of claims 1-3, wherein the bacterial PAL is a wild-type bacterial PAL or a mutant bacterial PAL.

5. The polynucleotide of any one of claims 1-4, comprising a codon-optimized coding region encoding a wild-type or mutant bacterial PAL as compared to a wild-type or reference coding region encoding the wild-type or mutant bacterial PAL.

6. The polynucleotide of claim 4 or claim 5, wherein the mutant bacterial PAL comprises a mutation at a position selected from C₅₀₃, C₅₆₅, or both C₅₀₃ and C₅₆₅.

7. The polynucleotide of any one of claims 1-6, wherein the coding region encoding the bacterial PAL comprises a sequence having at least 80% identity to a sequence selected from SEQ ID NOs:1-4.

8. The polynucleotide of any one of claims 1-7, wherein the polynucleotide further comprises a 5′ UTR.

9. The polynucleotide of claim 8, wherein the 5′ UTR comprises a sequence having at least 80% identity to a sequence of SEQ ID NO:8.

10. The polynucleotide of any one of claims 1-9, wherein the polynucleotide further comprises a 3′ UTR.

11. The polynucleotide of claim 10, wherein the 3′ UTR comprises a sequence having at least 80% identity to a sequence of SEQ ID NO:9.

12. The polynucleotide of any one of claims 1-11, further comprising a 3′ poly(A) sequence.

13. The polynucleotide of claim 12, wherein the poly(A) sequence comprises about 100 nucleotides.

14. A polynucleotide for expressing plant phenylalanine ammonia lyase (PAL) or a fragment thereof, wherein the polynucleotide comprises natural and chemically modified nucleotides, and wherein the polynucleotide is expressible to provide plant PAL or a fragment thereof having PAL activity.

15. The polynucleotide of claim 14, wherein the plant PAL is an Arabidopsis PAL, a Solanum PAL, or a Nicotiana PAL.

16. The polynucleotide of claim 15, wherein the plant PAL is an Arabidopsis PAL.

17. The polynucleotide of claim 16, wherein the plant PAL is an Arabidopsis thaliana PAL.

18. The polynucleotide of claim 15, wherein the plant PAL is a Solanum PAL.

19. The polynucleotide of claim 18, wherein the plant PAL is a Solanum lycopersicum PAL.

20. The polynucleotide of claim 15, wherein the plant PAL is a Nicotiana PAL.

21. The polynucleotide of claim 20, wherein the plant PAL is a Nicotiana tabacum PAL.

22. The polynucleotide of any one of claims 14-21, wherein the plant PAL is a wild-type plant PAL or a mutant plant PAL.

23. The polynucleotide of any one of claims 14-22, comprising a codon-optimized coding region encoding a wild-type or mutant plant PAL as compared to a wild-type or reference coding region encoding the wild-type or mutant plant PAL.

24. The polynucleotide of any one of claims 14-23, wherein the coding region encoding the plant PAL comprises a sequence having at least 80% identity to a sequence selected from SEQ ID NOs:5-7.

25. The polynucleotide of any one of claims 14-24, wherein the polynucleotide further comprises a 5′ UTR.

26. The polynucleotide of claim 25, wherein the 5′ UTR comprises a sequence having at least 80% identity to a sequence of SEQ ID NO:8.

27. The polynucleotide of any one of claims 14-26, wherein the polynucleotide further comprises a 3′ UTR.

28. The polynucleotide of claim 27, wherein the 3′ UTR comprises a sequence having at least 80% identity to a sequence of SEQ ID NO:9.

29. The polynucleotide of any one of claims 14-27, further comprising a 3′ poly(A) sequence.

30. The polynucleotide of claim 29, wherein the poly(A) sequence comprises about 100 nucleotides.

31. The polynucleotide of any one of claims 1-30, wherein the polynucleotide is an RNA molecule, wherein T is substituted with U.

32. The polynucleotide of claim 31, wherein the RNA molecule is an mRNA molecule or a self-replicating RNA molecule.

33. The polynucleotide of claim 32, wherein the RNA molecule is an mRNA molecule.

34. The polynucleotide of any one of claims 31-33, wherein the RNA molecule further comprises a 5′ cap.

35. The polynucleotide of claim 34, wherein the 5′ cap has a Cap 1 structure, a Cap 1 (^{m6}A) structure, a Cap 2 structure, or a Cap 0 structure.

36. The polynucleotide of any one of claims 33-35, wherein the chemically modified nucleotides include chemically modified nucleosides selected from 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine, 6-O-methylguanosine, and any combination thereof.

37. The polynucleotide of claim 36, wherein the chemically modified nucleosides are N¹-methylpseudouridines.

38. The polynucleotide of claim 36, wherein the chemically modified nucleosides are 5-methoxyuridines.

39. The polynucleotide of any one of claims 36-38, wherein the chemically modified nucleotides comprise 1-100% of the nucleotides that can be chemically modified.

40. The polynucleotide of any one of claims 36-38, wherein the chemically modified nucleotides comprise 50-100% of the nucleotides that can be chemically modified.

41. A DNA molecule encoding the polynucleotide of any one of claims 1-40.

42. The DNA molecule of claim 41, wherein the DNA molecule comprises a promoter.

43. The DNA molecule of claim 42, wherein the promoter is located 5′ of the 5′ UTR.

44. The DNA molecule of claim 42 or claim 43, wherein the promoter is a T7 promoter, a T3 promoter, or an SP6 promoter.

45. The DNA molecule of claim 42 or claim 43, wherein the promoter is an RNA polymerase II promoter.

46. A composition comprising the polynucleotide of any one of claims 1-40 and pharmaceutically acceptable carrier.

47. The composition of claim 46, wherein the pharmaceutically acceptable carrier comprises a lipid formulation.

48. The composition of claim 47, wherein the lipid formulation is selected from a transfection reagent, a lipoplex, a liposome, a lipid nanoparticle, a polymer-based carrier, an exosome, a lamellar body, a micelle, and an emulsion.

49. The composition of claim 48, wherein the lipid formulation is a liposome selected from a cationic liposome, a nanoliposome, a proteoliposome, a unilamellar liposome, a multilamellar liposome, a ceramide-containing nanoliposome, and a multivesicular liposome.

50. The composition of claim 48, wherein the lipid formulation is a lipid nanoparticle.

51. The composition of claim 50, wherein the lipid formulation or lipid nanoparticle encapsulates the polynucleotide.

52. The composition of any one of claims 47-51, wherein the lipid formulation comprises a cationic lipid.

53. The composition of claim 52, wherein the cationic lipid is an ionizable cationic lipid.

54. The composition of claim 52 or claim 53, wherein the lipid formulation further comprises at least one other lipid selected from the group consisting of anionic lipids, zwitterionic lipids, neutral lipids, steroids, polymer conjugated lipids, phospholipids, glycolipids, and combinations thereof.

55. A method for ameliorating, preventing, delaying onset, or treating a disease or condition associated with phenylketonuria, phenylalanine hydroxylase (PAH) deficiency, decreased metabolism of phenylalanine, or increased levels of phenylalanine in a subject in need thereof comprising: administering to the subject a polynucleotide of any one of claims 1-40 or a composition of any one of claims 46-54.

56. The method of claim 55, wherein the administering increases expression of the bacterial or plant PAL protein or a fragment thereof in the liver, serum, plasma, kidney, heart, muscle, brain, cerebrospinal fluid, lymph nodes, or any combination thereof, as compared with administering a control polynucleotide or a control composition or vehicle.

57. The method of claim 55 or claim 56, wherein the administering decreases blood phenylalanine levels, increases blood trans-cinnamic acid (tCA) levels, increases blood hippurate (HA) levels, or any combination thereof, as compared with administering a control polynucleotide or a control composition or vehicle.

58. The method of any one of claims 55-57, wherein the administration is intravenous, subcutaneous, intradermal, transdermal, intranasal, oral, sublingual, intraperitoneal, intramuscular, topical, or by a pulmonary route.

59. The method of any one of claims 55-58, wherein the administering comprises a therapeutically effective dose of from 0.01 mg/kg to 10 mg/kg.


SEQUENCE LISTING (56 sequences)

SEQ ID NO: 1 - DNA, 1818 bp, Anabaena variabilis
  source 1..1818: mol_type = other DNA
atgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggcctgcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgccctgcctgcacgcccccgcacccgcccctaggggaggcgggagcggcgagcagaaactgatcagcgaagaggacctggccgcaaacgacatcctggactacaaggatgacgacgataaggtgtga

SEQ ID NO: 2 - DNA, 1725 bp, Anabaena variabilis
  source 1..1725: mol_type = other DNA
atgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggcctgcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgccctgcctgcacgcccccgcacccgcccctagg

SEQ ID NO: 3 - DNA, 1816 bp, Anabaena variabilis
  source 1..1816: mol_type = other DNA
atgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggccagcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgcccagcctgcacgcccccgcacccgcccctaggggaggcgggagcggcgagcagaaactgatcagcgaagaggacctggccgcaaacgacatcctggactacaaggatgacgacgataaggtgt

SEQ ID NO: 4 - DNA, 1725 bp, Anabaena variabilis
  source 1..1725: mol_type = other DNA
atgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggccagcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgcccagcctgcacgcccccgcacccgcccctagg

SEQ ID NO: 5 - DNA, 2247 bp, Arabidopsis thaliana
  source 1..2247: mol_type = genomic DNA
atggaccagattgaggccatgctgtgcggcggcggcgagaagaccaaggtggccgtgacaaccaagaccctggccgaccctctgaactggggcctggccgccgaccagatgaagggcagccacctggacgaggtgaagaagatggtggaggagtacaggaggcccgtggtgaacctgggcggcgagacactgaccatcggccaggtggccgccatcagcaccgtgggcggcagcgtgaaggtggagctggccgagacaagcagggccggcgtgaaggccagcagcgactgggtgatggagagcatgaacaagggcaccgacagctacggcgtgaccaccggcttcggcgccaccagccaccggaggaccaagaacggcaccgccctgcagaccgagctgatcaggttcctgaacgccggcatcttcggcaacaccaaggagacatgccacaccctgccccagagcgccaccagggccgccatgctggtgagggtgaacaccctgctgcagggctacagcggcatcaggttcgagatcctggaggccatcaccagcctgctgaaccacaacatcagccccagcctgcccctgaggggcaccatcaccgccagcggcgacctggtgcccctgagctacatcgccggcctgctgaccggcaggcccaacagcaaggccaccggccccgacggcgagagcctgaccgccaaggaggccttcgagaaggccggcatcagcaccggcttcttcgacctgcagcccaaggagggcctggccctggtgaacggcaccgccgtgggcagcggcatggccagcatggtgctgttcgaggccaacgtgcaggccgtgctggccgaggtgctgagcgccatcttcgccgaggtgatgagcggcaagcccgagttcaccgaccacctgacccacaggctgaagcaccaccccggccagatcgaggccgccgccatcatggagcacatcctggacggcagcagctacatgaagctggcccagaaggtgcacgagatggaccctctgcagaagcccaagcaggacaggtacgccctgaggaccagccctcagtggctgggccctcagatcgaggtgatcaggcaggccaccaagagcatcgagagggagatcaacagcgtgaacgacaatcccctgatcgacgtgagcaggaacaaggccatccacggcggcaacttccagggcacccctatcggcgtgagcatggacaacaccaggctggccatcgccgccatcggcaagctgatgttcgcccagttcagcgagctggtgaacgacttctacaacaacggcctgcccagcaacctgaccgccagcagcaaccccagcctggactacggcttcaagggcgccgagatcgctatggccagctactgcagcgagctgcagtacctggccaaccccgtgaccagccacgtgcagagcgccgagcagcacaaccaggacgtgaacagcctgggcctgatcagcagcaggaagaccagcgaggccgtggacatcctgaagctgatgagcaccaccttcctggtgggcatctgccaggccgtggacctgaggcacctggaggagaacctgaggcagaccgtgaagaacaccgtgagccaggtggccaagaaggtgctgaccaccggcatcaacggcgagctgcaccccagcaggttctgcgagaaggacctgctgaaggtggtggacagggagcaggtgttcacctacgtggacgacccctgcagcgccacctaccctctgatgcagaggctgaggcaggtgatcgtggaccacgccctgagcaacggcgagacagagaagaacgccgtgaccagcatcttccagaagatcggcgccttcgaggaggagctgaaggccgtgctgcccaaggaggtggaggccgccagggccgcctacggcaacggcaccgcccctatccccaaccggatcaaggagtgcaggagctaccctctgtaccggttcgtgagggaggagctgggcaccaagctgctgaccggcgagaaggtggtgagccccggcgaggagttcgacaaggtgttcaccgccatgtgcgagggcaagctgatcgaccctctgatggactgcctgaaggagtggaacggcgcccctatccccatctgccctaggggaggcgggagcggcgagcagaaactgatcagcgaagaggacctggccgcaaacgacatcctggactacaaggacgacgacgacaagtag

SEQ ID NO: 6 - DNA, 2259 bp, Solanum lycopersicum
  source 1..2259: mol_type = genomic DNA
atggcctctagcatcgtgcagaacggccacgtgaatggcgaggctatggacctgtgcaagaagtccatcaacgtgaacgaccctctgaactgggagatggccgccgagagcctgaggggcagccacctggacgaggtgaagaagatggtggacgagttcaggaagcccatcgtgaagctgggcggcgagacactgaccgtggcccaggtggccagcatcgccaacgtggacaacaagagcaacggcgtgaaggtggagctgagcgagagcgccagggccggcgtgaaggccagcagcgactgggtgatggacagcatgggcaagggcaccgacagctacggcgtgaccaccggcttcggcgccaccagccacaggaggaccaagaacggcggcgccctgcagaaggagctgatcaggttcctgaacgccggcgtgttcggcaacggcaccgagagcagccacaccctgccccacagcgccaccagggccgccatgctggtgaggatcaacaccctgctgcagggctacagcggcatcaggttcgagatcctggaggccatcaccaagctgatcaacagcaacatcaccccttgcctgcccctgaggggcaccatcaccgccagcggcgacctggtgcccctgagctacatcgccggcctgctgaccggcaggcccaacagcaaggccgtgggccccaacggcgagaagctgaacgccgaggaggccttccacgtggccggcgtgaccagcggcttcttcgagctgcagcccaaggagggcctggccctggtgaacggcaccgccgtgggcagcggcatggccagcatggtgctgttcgagagcaacatcctggccgtgatgagcgaggtgctgagcgccatcttcgccgaggtgatgaacggcaagcccgagttcaccgactacctgacccacaagctgaagcaccaccccggccagatcgaggccgccgccatcatggagcacatcctggacggcagcagctacgtgaaggaggcccagaagctgcacgagatggaccctctgcagaagcccaagcaggacaggtacgccctgaggaccagccctcagtggctgggccctcagatcgaggtgatcagggccgccaccaagatgatcgagagggagatcaacagcgtgaacgacaatcccctgatcgacgtgagcaggaacaaggccctgcacggcggcaacttccagggcacccctatcggcgtgagcatggacaacaccaggctggccctggccagcatcggcaagctgatgttcgcccagttcagcgagctggtgaacgactactacaacaacggcctgcccagcaacctgaccgccggcaggaaccccagcctggactacggcttcaagggcgccgagatcgctatggccagctactgcagcgagctgcagttcctggccaaccccgtgaccaaccacgtgcagagcgccgagcagcacaaccaggacgtgaacagcctgggcctgatcagcgccaggaagaccgccgaggccgtggacatcctgaagctgatgagcagcacctacctggtggccctgtgccaggccatcgacctgaggcacctggaggagaacctgaagaacgccgtgaagaacaccgtgagccaggtggccaagaagaccctggctatgggcgccaacggcgagctgcaccccgccaggttctgcgagaaggagctgctgcaggtggtggagagggagtacctgttcacctacgccgacgacccctgcagcagcacctaccctctgatgcagaagctgaggcaggtgctggtggaccacgccatgaagaacggcgagagcgagaagaacgtgaacagcagcatcttccagaagatcgtggccttcgaggacgagctgaaggccgtgctgcccaaggaggtggagagcgccagggccgtggtggagagcggcaaccccgccatccccaacaggatcaccgagtgcaggagctaccctctgtaccggctggtgaggcaggaggtgggcaccgagctgctgaccggcgagaaggtgaggagccccggcgaggagatcgacaaggtgttcaccgccttctgcaacggccagatcatcgaccctctgctggagtgcctgaagtcctggaacggcgcccctatccccatctgccctaggggaggcgggagcggcgagcagaaactgatcagcgaagaggacctggccgcaaacgacatcctggactacaaggacgacgacgacaagtag

SEQ ID NO: 7 - DNA, 2232 bp, Nicotiana tabacum
  source 1..2232: mol_type = genomic DNA
atggccggcgtggcccagaacggccaccaggagatggacttctgcgttaaggtggaccctctgaactgggagatggccgccgacagcctgaagggcagccacctggacgaggtgaagaagatggtggccgagttcaggaagcccgtggtgaagctgggcggcgagacactgaccgtggcccaggtggccgccatcgccgccaaggacaacgccaagaccgtgaaggtggagctgagcgagggcgccagggccggcgtgaaggccagcagcgactgggtgatggacagcatgagcaagggcaccgacagctacggcgtgaccaccggcttcggcgccaccagccacaggaggaccaagaacggcggcgccctgcagaaggagctgatcaggttcctgaacgccggcgtgttcggcaacggcaccgagagctgccacaccctgccccagagcggcaccagggccgccatgctggtgaggatcaacaccctgctgcagggctacagcggcatcaggttcgagatcctggaggccatcaccaagctgctgaaccacaacgtgaccccttgcctgcccctgaggggcaccatcaccgccagcggcgacctggtgcccctgagctacatcgccggcctgctgaccggccggcccaacagcaaggccatcggccccaacggcgagacactgaacgccgaggaggccttcagggtggccggcgtgaacagcggcttcttcgagctgcagcccaaggagggcctggccctggtgaacggcaccgccgtgggcagcggcctggccagcatggtgctgttcgacgccaacatcctggccgtgttcagcgaggtgctgagcgccatcttcgccgaggtgatgaacggcaagcccgagttcaccgaccacctgacccacaagctgaagcaccaccccggccagatcgaggccgccgccatcatggagcacatcctggacggcagcagctacgtgaaggcccctcagaagctgcacgagacagaccctctgcagaagcccaagcaggacaggtacgccctgaggaccagccctcagtggctgggccctcagatcgaggtgatcaggagcgccaccaagatgatcgagagggagatcaacagcgtgaacgacaatcccctgatcgacgtgagcaggaacaaggccctgcacggcggcaacttccagggcacccctatcggcgtgagcatggacaacgccaggctggccctggccagcatcggcaagctgatgttcgcccagttcagcgagctggtgaacgactactacaacaacggcctgcccagcaacctgaccgccggcaggaaccccagcctggactacggcttcaagggcagcgagatcgctatggccagctactgcagcgagctgcagttcctggccaaccccgtgaccaaccacgtgcagagcgccgagcagcacaaccaggacgtgaacagcctgggcctgatcagcgccaggaagaccgccgaggccgtggacatcctgaagctgatgagcagcacctacctggtggccctgtgccaggccatcgacctgaggcacctggaggagaacctgaggaacgccgtgaagaacaccgtgagccaggtggccaagaggaccctgacaatgggcgccaacggcgagctgcaccccagcaggttctgcgagaaggacctgctgagggtggtggacagggagtacgtgttcaggtacgccgacgacgcctgcagcgccaactaccctctgatgcagaagctgaggcaggtgctggtggaccacgccctggagaacggcgagaacgagaagaacgccaacagcagcatcttccagaagatcctggccttcgagggcgagctgaaggccgtgctgcccaaggaggtggagagcgccaggatcagcctggagaacggcaaccccgccatcgccaacaggatcaaggagtgcaggagctaccctctgtaccggttcgtgagggaggagctgggcgccgagctgctgaccggcgagaaggtgaggagccccggcgaggagtgcgacaaggtgttcaccgccatgtgcaacggccagatcatcgacagcctgctggagtgcctgaaggagtggaacggcgcccctctgcccatctgccctaggggaggcgggagcggcgagcagaaactgatcagcgaagaggacctggccgcaaacgacatcctggactacaaggacgacgacgacaagtag

SEQ ID NO: 8 - DNA, 146 bp, Tobacco etch virus
  source 1..146: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccacc

SEQ ID NO: 9 - DNA, 168 bp, Xenopus laevis
  source 1..168: mol_type = other DNA
tcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 10 - DNA, 2233 bp, Anabaena variabilis
  source 1..2233: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggcctgcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgccctgcctgcacgcccccgcacccgcccctaggggaggcgggagcggcgagcagaaactgatcagcgaagaggacctggccgcaaacgacatcctggactacaaggatgacgacgataaggtgtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 2230 bp, Anabaena variabilis
  source 1..2230: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggcctgcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgccctgcctgcacgcccccgcacccgcccctaggggaggcgggagcggcgagcagaaactgatcagcgaagaggacctggccgcaaacgacatcctggactacaaggatgacgacgataaggtgtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 2233 bp, Anabaena variabilis
  source 1..2233: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggccagcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgcccagcctgcacgcccccgcacccgcccctaggggaggcgggagcggcgagcagaaactgatcagcgaagaggacctggccgcaaacgacatcctggactacaaggatgacgacgataaggtgtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 2230 bp, Anabaena variabilis
  source 1..2230: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcaagaccctgagccaggcccagagcaagaccagcagccagcagttcagcttcaccggcaacagcagcgccaacgtgatcatcggcaaccagaagctgaccatcaacgacgtggccagggtggcccggaacggcaccctggtgagcctgaccaacaacaccgacatcctgcagggcatccaggccagctgcgactacatcaacaacgccgtggagagcggcgagcccatctacggcgtgaccagcggcttcggcggaatggccaacgtggccatcagcagggagcaggccagcgagctgcagaccaacctggtgtggttcctgaagaccggagccggcaacaagctgccactggccgacgtgagagcagccatgctcctgagggccaacagccacatgagaggcgccagcggcatcaggctggagctgatcaagaggatggagatcttcctgaacgccggcgtgaccccatacgtgtacgagttcggcagcatcggcgccagcggcgacctggtgcccctgagctacatcaccggcagcctgatcggcctggaccccagcttcaaggtggacttcaacggcaaggagatggacgccccaaccgccctgaggcagctgaacctgagccccctgaccctgctgcccaaggagggcctggcaatgatgaacggcaccagcgtgatgaccggcatcgccgccaactgcgtgtacgacacccagatcctgaccgccatcgcaatgggcgtgcacgccctggacatccaggccctgaacggcaccaaccagagcttccaccccttcatccacaacagcaagccacaccccggacagctgtgggccgcagaccagatgatcagcctgctcgccaacagccagctggtgagggacgagctggacggcaagcacgactacagggaccacgagctgatccaggacaggtacagcctgaggtgcctgccccagtacctgggcccaatcgtggacggcatcagccagatcgccaagcagatcgagatcgagatcaacagcgtgaccgacaacccactgatcgacgtggacaaccaggccagctaccacggcggaaacttcctgggccagtacgtgggaatgggcatggaccacctgaggtactacatcggcctgctcgccaagcacctggacgtgcagatcgccctgctcgccagcccagagttcagcaacggactgccacccagcctcctgggcaacagggagcggaaggtgaacatgggcctgaagggactgcagatctgcggcaacagcatcatgccactcctgaccttctacggcaacagcatcgccgacaggttccccacccacgccgagcagttcaaccagaacatcaacagccagggctacaccagcgccaccctggccaggcggagcgtggacatcttccagaactacgtggccatcgcactgatgttcggcgtgcaggccgtggacctgaggacctacaagaagaccggccactacgacgccagggccagcctgagccccgccaccgagaggctgtacagcgccgtgaggcacgtggtcggccagaagcccaccagcgacaggccctacatctggaacgacaacgagcagggcctggacgagcacatcgccaggatcagcgccgacatcgccgcaggcggagtgatcgtgcaggccgtgcaggacatcctgcccagcctgcacgcccccgcacccgcccctaggggaggcgggagcggcgagcagaaactgatcagcgaagaggacctggccgcaaacgacatcctggactacaaggatgacgacgataaggtgtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 2658 bp, Arabidopsis thaliana
  source 1..2658: mol_type = genomic DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatggaccagattgaggccatgctgtgcggcggcggcgagaagaccaaggtggccgtgacaaccaagaccctggccgaccctctgaactggggcctggccgccgaccagatgaagggcagccacctggacgaggtgaagaagatggtggaggagtacaggaggcccgtggtgaacctgggcggcgagacactgaccatcggccaggtggccgccatcagcaccgtgggcggcagcgtgaaggtggagctggccgagacaagcagggccggcgtgaaggccagcagcgactgggtgatggagagcatgaacaagggcaccgacagctacggcgtgaccaccggcttcggcgccaccagccaccggaggaccaagaacggcaccgccctgcagaccgagctgatcaggttcctgaacgccggcatcttcggcaacaccaaggagacatgccacaccctgccccagagcgccaccagggccgccatgctggtgagggtgaacaccctgctgcagggctacagcggcatcaggttcgagatcctggaggccatcaccagcctgctgaaccacaacatcagccccagcctgcccctgaggggcaccatcaccgccagcggcgacctggtgcccctgagctacatcgccggcctgctgaccggcaggcccaacagcaaggccaccggccccgacggcgagagcctgaccgccaaggaggccttcgagaaggccggcatcagcaccggcttcttcgacctgcagcccaaggagggcctggccctggtgaacggcaccgccgtgggcagcggcatggccagcatggtgctgttcgaggccaacgtgcaggccgtgctggccgaggtgctgagcgccatcttcgccgaggtgatgagcggcaagcccgagttcaccgaccacctgacccacaggctgaagcaccaccccggccagatcgaggccgccgccatcatggagcacatcctggacggcagcagctacatgaagctggcccagaaggtgcacgagatggaccctctgcagaagcccaagcaggacaggtacgccctgaggaccagccctcagtggctgggccctcagatcgaggtgatcaggcaggccaccaagagcatcgagagggagatcaacagcgtgaacgacaatcccctgatcgacgtgagcaggaacaaggccatccacggcggcaacttccagggcacccctatcggcgtgagcatggacaacaccaggctggccatcgccgccatcggcaagctgatgttcgcccagttcagcgagctggtgaacgacttctacaacaacggcctgcccagcaacctgaccgccagcagcaaccccagcctggactacggcttcaagggcgccgagatcgctatggccagctactgcagcgagctgcagtacctggccaaccccgtgaccagccacgtgcagagcgccgagcagcacaaccaggacgtgaacagcctgggcctgatcagcagcaggaagaccagcgaggccgtggacatcctgaagctgatgagcaccaccttcctggtgggcatctgccaggccgtggacctgaggcacctggaggagaacctgaggcagaccgtgaagaacaccgtgagccaggtggccaagaaggtgctgaccaccggcatcaacggcgagctgcaccccagcaggttctgcgagaaggacctgctgaaggtggtggacagggagcaggtgttcacctacgtggacgacccctgcagcgccacctaccctctgatgcagaggctgaggcaggtgatcgtggaccacgccctgagcaacggcgagacagagaagaacgccgtgaccagcatcttccagaagatcggcgccttcgaggaggagctgaaggccgtgctgcccaaggaggtggaggccgccagggccgcctacggcaacggcaccgcccctatccccaaccggatcaaggagtgcaggagctaccctctgtaccggttcgtgagggaggagctgggcaccaagctgctgaccggcgagaaggtggtgagccccggcgaggagttcgacaaggtgttcaccgccatgtgcgagggcaagctgatcgaccctctgatggactgcctgaaggagtggaacggcgcccctatccccatctgccctaggggaggcgggagcggcgagcagaaactgatcagcgaagaggacctggccgcaaacgacatcctggactacaaggacgacgacgacaagtagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - DNA, 2671 bp, Solanum lycopersicum
  source 1..2671: mol_type = genomic DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatggcctctagcatcgtgcagaacggccacgtgaatggcgaggctatggacctgtgcaagaagtccatcaacgtgaacgaccctctgaactgggagatggccgccgagagcctgaggggcagccacctggacgaggtgaagaagatggtggacgagttcaggaagcccatcgtgaagctgggcggcgagacactgaccgtggcccaggtggccagcatcgccaacgtggacaacaagagcaacggcgtgaaggtggagctgagcgagagcgccagggccggcgtgaaggccagcagcgactgggtgatggacagcatgggcaagggcaccgacagctacggcgtgaccaccggcttcggcgccaccagccacaggaggaccaagaacggcggcgccctgcagaaggagctgatcaggttcctgaacgccggcgtgttcggcaacggcaccgagagcagccacaccctgccccacagcgccaccagggccgccatgctggtgaggatcaacaccctgctgcagggctacagcggcatcaggttcgagatcctggaggccatcaccaagctgatcaacagcaacatcaccccttgcctgcccctgaggggcaccatcaccgccagcggcgacctggtgcccctgagctacatcgccggcctgctgaccggcaggcccaacagcaaggccgtgggccccaacggcgagaagctgaacgccgaggaggccttccacgtggccggcgtgaccagcggcttcttcgagctgcagcccaaggagggcctggccctggtgaacggcaccgccgtgggcagcggcatggccagcatggtgctgttcgagagcaacatcctggccgtgatgagcgaggtgctgagcgccatcttcgccgaggtgatgaacggcaagcccgagttcaccgactacctgacccacaagctgaagcaccaccccggccagatcgaggccgccgccatcatggagcacatcctggacggcagcagctacgtgaaggaggcccagaagctgcacgagatggaccctctgcagaagcccaagcaggacaggtacgccctgaggaccagccctcagtggctgggccctcagatcgaggtgatcagggccgccaccaagatgatcgagagggagatcaacagcgtgaacgacaatcccctgatcgacgtgagcaggaacaaggccctgcacggcggcaacttccagggcacccctatcggcgtgagcatggacaacaccaggctggccctggccagcatcggcaagctgatgttcgcccagttcagcgagctggtgaacgactactacaacaacggcctgcccagcaacctgaccgccggcaggaaccccagcctggactacggcttcaagggcgccgagatcgctatggccagctactgcagcgagctgcagttcctggccaaccccgtgaccaaccacgtgcagagcgccgagcagcacaaccaggacgtgaacagcctgggcctgatcagcgccaggaagaccgccgaggccgtggacatcctgaagctgatgagcagcacctacctggtggccctgtgccaggccatcgacctgaggcacctggaggagaacctgaagaacgccgtgaagaacaccgtgagccaggtggccaagaagaccctggctatgggcgccaacggcgagctgcaccccgccaggttctgcgagaaggagctgctgcaggtggtggagagggagtacctgttcacctacgccgacgacccctgcagcagcacctaccctctgatgcagaagctgaggcaggtgctggtggaccacgccatgaagaacggcgagagcgagaagaacgtgaacagcagcatcttccagaagatcgtggccttcgaggacgagctgaaggccgtgctgcccaaggaggtggagagcgccagggccgtggtggagagcggcaaccccgccatccccaacaggatcaccgagtgcaggagctaccctctgtaccggctggtgaggcaggaggtgggcaccgagctgctgaccggcgagaaggtgaggagccccggcgaggagatcgacaaggtgttcaccgccttctgcaacggccagatcatcgaccctctgctggagtgcctgaagtcctggaacggcgcccctatccccatctgccctaggggaggcgggagcggcgagcagaaactgatcagcgaagaggacctggccgcaaacgacatcctggactacaaggacgacgacgacaagtagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - DNA, 2644 bp, Nicotiana tabacum
  source 1..2644: mol_type = genomic DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatggccggcgtggcccagaacggccaccaggagatggacttctgcgttaaggtggaccctctgaactgggagatggccgccgacagcctgaagggcagccacctggacgaggtgaagaagatggtggccgagttcaggaagcccgtggtgaagctgggcggcgagacactgaccgtggcccaggtggccgccatcgccgccaaggacaacgccaagaccgtgaaggtggagctgagcgagggcgccagggccggcgtgaaggccagcagcgactgggtgatggacagcatgagcaagggcaccgacagctacggcgtgaccaccggcttcggcgccaccagccacaggaggaccaagaacggcggcgccctgcagaaggagctgatcaggttcctgaacgccggcgtgttcggcaacggcaccgagagctgccacaccctgccccagagcggcaccagggccgccatgctggtgaggatcaacaccctgctgcagggctacagcggcatcaggttcgagatcctggaggccatcaccaagctgctgaaccacaacgtgaccccttgcctgcccctgaggggcaccatcaccgccagcggcgacctggtgcccctgagctacatcgccggcctgctgaccggccggcccaacagcaaggccatcggccccaacggcgagacactgaacgccgaggaggccttcagggtggccggcgtgaacagcggcttcttcgagctgcagcccaaggagggcctggccctggtgaacggcaccgccgtgggcagcggcctggccagcatggtgctgttcgacgccaacatcctggccgtgttcagcgaggtgctgagcgccatcttcgccgaggtgatgaacggcaagcccgagttcaccgaccacctgacccacaagctgaagcaccaccccggccagatcgaggccgccgccatcatggagcacatcctggacggcagcagctacgtgaaggcccctcagaagctgcacgagacagaccctctgcagaagcccaagcaggacaggtacgccctgaggaccagccctcagtggctgggccctcagatcgaggtgatcaggagcgccaccaagatgatcgagagggagatcaacagcgtgaacgacaatcccctgatcgacgtgagcaggaacaaggccctgcacggcggcaacttccagggcacccctatcggcgtgagcatggacaacgccaggctggccctggccagcatcggcaagctgatgttcgcccagttcagcgagctggtgaacgactactacaacaacggcctgcccagcaacctgaccgccggcaggaaccccagcctggactacggcttcaagggcagcgagatcgctatggccagctactgcagcgagctgcagttcctggccaaccccgtgaccaaccacgtgcagagcgccgagcagcacaaccaggacgtgaacagcctgggcctgatcagcgccaggaagaccgccgaggccgtggacatcctgaagctgatgagcagcacctacctggtggccctgtgccaggccatcgacctgaggcacctggaggagaacctgaggaacgccgtgaagaacaccgtgagccaggtggccaagaggaccctgacaatgggcgccaacggcgagctgcaccccagcaggttctgcgagaaggacctgctgagggtggtggacagggagtacgtgttcaggtacgccgacgacgcctgcagcgccaactaccctctgatgcagaagctgaggcaggtgctggtggaccacgccctggagaacggcgagaacgagaagaacgccaacagcagcatcttccagaagatcctggccttcgagggcgagctgaaggccgtgctgcccaaggaggtggagagcgccaggatcagcctggagaacggcaaccccgccatcgccaacaggatcaaggagtgcaggagctaccctctgtaccggttcgtgagggaggagctgggcgccgagctgctgaccggcgagaaggtgaggagccccggcgaggagtgcgacaaggtgttcaccgccatgtgcaacggccagatcatcgacagcctgctggagtgcctgaaggagtggaacggcgcccctctgcccatctgccctaggggaggcgggagcggcgagcagaaactgatcagcgaagaggacctggccgcaaacgacatcctggactacaaggacgacgacgacaagtagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 18 - protein, 665 aa, Arabidopsis thaliana
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDSYGVTTGFGATSHRRTKNGVALQKELIRFLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAIMEHILDGSSYMKLAQKLHEMDPLQKPKQDRYTRLAIAAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALINGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

SEQ ID NO: 19 - protein, 704 aa, Solanum lycopersicum
MDLCKKSINDPLNWEMAADSLRGSHLDEVKKMVDEFRKPIVKLGGETLSVAQVASIANVDDKSNGVKVELSESARAGVKASSDWVMDSMSKGTDSYGVTAGFGATSHRRTKNGGALQKELIRFLNAGVFGNGIESFHTLPHSATRAAMLVRINTLLQGYSGIRFEILEAITKLINSNITPCLPLRGTITASGDLVPLSYIAGLLTGRPNSKAVGPNGEKLNAEEAFCVAGISGGFFELQPKEGLALVNGTAVGSAMASIVLFESNIFAVMSEVLSAIFTEVMNGKPEFTDYLTHKLKHHPGQIEAAAIMEHILDGSSYVKVAQKLHEMDPLQKPKQDRYALRTSPQWLGPQIEVIRAATKMIEREINSVNDNPLIDVSRNKALHGGNFQGTPIGVSMDNTRLALASIGKLMFAQFSELVNDYYNNGLPSNLTAGRNPSLDYGFKGAEIAMASYCSELQFLANPVTNHVQSAEQHNQDVNSLGLISARKTAKAVDILKIMSSTYLVALCQAIDLRHLEENLKSVVKNTVSQVAKRTLTMGANGELHPARFSEKELLRVVDREYLFAYADDPCSSNYPLMQKLRQVLVDQAMKNGESEKNVNSSIFQKIGAFEDELIAVLPKEVESVRAVFESGNPLIRNRITECRSYPLYRLVREELGTELLTGEKVRSPGEEIDKVFTAICNGQIIDPLLECLKSWNGAPLPIC

SEQ ID NO: 20 - protein, 715 aa, Nicotiana tabacum
MASNGHVNGGENFELCKKSADPLNWEMAAESLRGSHLDEVKKMVSEFRKPMVKLGGESLTVAQVAAIAVRDKSANGVKVELSEEARAGVKASSDWVMDSMNKGTDSYGVTTGFGATSHRRTKNGGALQKELIRFLNAGVFGNGTETSHTLPHSATRAAMLVRINTLLQGYSGIRFEILEAITKLINSNITPCLPLRGTITASGDLVPLSYIAGLLTGRPNSKAVGPNGETLNAEEAFRVAGVNGGFFELQPKEGLALVNGTAVGSGMASMVLFDSNILAVMSEVLSAIFAEVMNGKPEFTDHLTHKLKHHPGQIEAAAIMEHILDGSSYVKAAQKLHEMDPLQKPKQDRYALRTSPQWLGPQIEVIRAATKMIEREINSVNDNPLIDVSRNKALHGGNFQGTPIGVSMDNARLALASIGKLMFAQFSELVNDYYNNGLPSNLTASRNPSLDYGFKGAEIAMASYCSELQFLANPVTNHVQSAEQHNQDVNSLGLISARKTAEAVDILKLMSSTYLVALCQAIDLRHLEENLKNAVKNTVSQVAKRTLTMGANGELHPARFCEKELLRIVDREYLFAYADDPCSCNYPLMQKLRQVLVDHAMNNGESEKNVNSSIFQKIGAFEDELKAVLPKEVESARAALESGNPAIPNRITECRSYPLYRFVRKELGTELLTGEKVRSPGEECDKVFTAMCNGQIIDPMLECLKSWNGAPLPIC

SEQ ID NO: 21-23 - skipped (3 entries)

SEQ ID NO: 24 - RNA, 129 nt, Tobacco etch virus
  source 1..129: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 25 - RNA, 28 nt, Arabidopsis thaliana
  source 1..28: mol_type = other RNA
attattacatcaaaacaaaaagccgcca

SEQ ID NO: 26 - RNA, 89 nt, Homo sapiens
  source 1..89: mol_type = other RNA
aattattggttaaagaagtatattagtgctaatttccctccgtttgtcctagcttttctcttctgtcaaccccacacgcctttggcaca

SEQ ID NO: 27 - RNA, 21 nt, Synechocystis sp.
  source 1..21: mol_type = other RNA
aacttaaaaaaaaaaatcaaa

SEQ ID NO: 28 - RNA, 53 nt, Mus musculus
  source 1..53: mol_type = other RNA
cacatttgcttctgacatagttgtgttgactcacaaccccagaaacagacatc

SEQ ID NO: 29 - RNA, 50 nt, Homo sapiens
  source 1..50: mol_type = other RNA
acatttgcttctgacacaactgtgttcactagcaacctcaaacagacacc

SEQ ID NO: 30 - RNA, 46 nt, Mus musculus
  source 1..46: mol_type = other RNA
tgcacacagatcacctttcctatcaaccccactagcctctggcaaa

SEQ ID NO: 31 - RNA, 58 nt, Homo sapiens
  source 1..58: mol_type = other RNA
ataaaaagaccagcagatgccccacagcactgctcttccagaggcaagaccaaccaag

SEQ ID NO: 32 - RNA, 177 nt, Homo sapiens
  source 1..177: mol_type = other RNA
agacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttggcagg

SEQ ID NO: 33 - RNA, 93 nt, Homo sapiens
  source 1..93: mol_type = other RNA
agataaaaagccagctccagcaggcgctgctcactcctccccatcctctccctctgtccctctgtccctctgaccctgcactgtcccagcacc

SEQ ID NO: 34 - RNA, 30 nt, Homo sapiens
  source 1..30: mol_type = other RNA
tatatccgtggtttcctgctacctccaacc

SEQ ID NO: 35 - RNA, 34 nt, Homo sapiens
  source 1..34: mol_type = other RNA
ggcaccaccactgacctgggacagtgaatcgaca

SEQ ID NO: 36 - RNA, 79 nt, Homo sapiens
  source 1..79: mol_type = other RNA
attcatgaaaatccactactccagacagacggctttggaatccaccagctacatccagctccctgaggcagagttgaga

SEQ ID NO: 37 - RNA, 116 nt, Homo sapiens
  source 1..116: mol_type = other RNA
aatattagagtctcaacccccaataaatataggactggagatgtctgaggctcattctgccctcgagcccaccgggaacgaaagagaagctctatctcccctccaggagcccagct

SEQ ID NO: 38 - RNA, 79 nt, Homo sapiens
  source 1..79: mol_type = other RNA
aggatgggaactaggagtggcagcaatcctttctttcagctggagtgctcctcaggagccagccccacccttagaaaag

SEQ ID NO: 39 - RNA, 111 nt, Homo sapiens
  source 1..111: mol_type = other RNA
agggggagccctataattggacaagtctgggatccttgagtcctactcagccccagcggaggtgaaggacgtccttccccaggagccgactggccaatcacaggcaggaag

SEQ ID NO: 40 - RNA, 223 nt, Homo sapiens
  source 1..223: mol_type = other RNA
agacgggtggggcggggcccaactgtccccagctccttcagccctttctgtccctcccagtgaggccagctgcggtgaagagggtgctctcttgcctggagttccctctgctacggctgccccctcccagccctggcccactaagccagacccagctgtcgccattcccacttctggtcctgccacctcctgagctgccttcccgcctggtctgggtagagtc

SEQ ID NO: 41 - RNA, 119 nt, Homo sapiens
  source 1..119: mol_type = other RNA
tctgccccaccctgtcctctggaacctctgcgagatttagaggaaagaaccagttttcaggcggattgcctcagatcacactatctccacttgcccagccctgtggaagattagcggcc

SEQ ID NO: 42 - RNA, 158 nt, Homo sapiens
  source 1..158: mol_type = other RNA
ctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 43 - RNA, 166 nt, Homo sapiens
  source 1..166: mol_type = other RNA
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccat

SEQ ID NO: 44 - RNA, 143 nt, Homo sapiens
  source 1..143: mol_type = other RNA
acgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 45 - RNA, 170 nt, Mus musculus
  source 1..170: mol_type = other RNA
acacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctac

SEQ ID NO: 46 - RNA, 110 nt, Homo sapiens
  source 1..110: mol_type = other RNA
gctggagcctcggtagccgttcctcctgcccgctgggcctcccaacgggccctcctcccctccttgcaccggcccttcctggtctttgaataaagtctgagtgggcagca

SEQ ID NO: 47 - RNA, 142 nt, Mus musculus
  source 1..142: mol_type = other RNA
accccctttcctgctcttgcctgtgaacaatggttaattgttcccaagagagcatctgtcagttgttggcaaaatgataaagacatttgaaaatctgtcttctgacaaataaaaagcatttatttcactgcaatgatgtttt

SEQ ID NO: 48 - RNA, 134 nt, Homo sapiens
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 49 - RNA, 106 nt, Homo sapiens
  source 1..106: mol_type = other RNA
tggcatccctgtgacccctccccagtgcctctcctggccctggaagttgccactccagtgcccaccagccttgtcctaataaaattaagttgcatcattttgtctg

SEQ ID NO: 50 - RNA, 80 nt, Homo sapiens
  source 1..80: mol_type = other RNA
aatgttcttattctttgcacctcttcctatttttggtttgtgaacagaagtaaaaataaatacaaactacttccatctca

SEQ ID NO: 51 - RNA, 178 nt, Homo sapiens
  source 1..178: mol_type = other RNA
ccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 52 - RNA, 101 nt, Homo sapiens
  source 1..101: mol_type = other RNA
aacctacctgccctgcccccgtcccctcccttccttatttattcctgctgccccagaacataggtcttggaataaaatggctggttcttttgttttccaaa

SEQ ID NO: 53 - RNA, 234 nt, Homo sapiens
  source 1..234: mol_type = other RNA
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactcaataaatacatgttaaatggatgaatgaattcctctgaaactct

SEQ ID NO: 54 - RNA, 171 nt, Homo sapiens
  source 1..171: mol_type = other RNA
gcaccccagctggggccaggctgggtcgccctggactgtgtgctcaggagccctgggaggctctggagcccactgtacttgctcttgatgcctggcggggtggggtggggggggtgctgggcccctgcctctctgcaggtccctaataaagctgtgtggcagtctgactcc

SEQ ID NO: 55 - RNA, 110 nt, Homo sapiens
  source 1..110: mol_type = other RNA
gattcgtcagtagggttgtaaaggtttttcttttcctgagaaaacaaccttttgttttctcaggttttgctttttggcctttccctagctttaaaaaaaaaaaagcaaaa

SEQ ID NO: 56 - RNA, 1932 nt, Homo sapiens
  source 1..1932: mol_type = genomic RNA
ggacgcctcaggcaccggagccagaccctcccaagaccacccaggccttcctcaaggactctgcctcagacctcagacaggccaccaacgctgttcatcttcatttccccaaggagacttctttctttgtgccttgatgtttgagagttcttcgagcaaacagtggttttgcaatgtctcacaggccctgtttttgtttttgtttttgttttgttttgttttgttctttttttaaatgcaaccaaagtagagtcaacctgctcggcagatgtacttggattctctgaatcgctattctgtttggagagttcctttgggtcttaagcagccagagtacatggaaatgagattatgtcagatctggagaaacaagcaggtgttgggaaatatgtgacttgacatgataagggctgggaatccagaaatcaatagtgagatccatgaaatcaaaccctgaccagtgtgaaaatgtagccttttggacagtaagcctgcaagtctagtgagaactcagagaaagctgaccattctggtctgaagataggcagcgcatcacaggcaagaatatcgaagtcagtagtaggacaggggtcacatcagataccagctcaaattgcactagctatctagaacagttttctccaggtttgcctgagccttgatgcataccatcgccctctgctggtcgcagcagagataagcaagggctgaaaatggaggcaatcctttcccaaggccctgaaagttgtttttcatggtttcaaactgaatttggctcatttgtaactaactgatcacggtgcctggttacactggctgccaagaaggagcgcatgcaatctgattcagtgctctcttcacatcagtttcctgcctccctccctcatctgcggacagcatcctatctcatcaggcttccctgtgtgtcacaaagtagcagccaccaagcaaatatattccttgaattagcacacctgggtgggccatgtgcgcaccaaggaaacaggtgctatagggagcgccaggccaggcttgtctcttaactgtctcgttcttcagtgagagtgggaaagctgtccggagctcccgcgcaggagcctgggtacccacgcagcgagtcaagggagttttcggagccagagagagaaagatgtgaaggctgtggagtaaggctgaaaccagcctcctgccctatagtcccacactgcagggggtgcgactttaaaacagaacttcaagttgttaacactcacaagcattgcattactgtgaaggaagtagccgcatccataacaggatgtgatggtctacagcttttcctttaaaagctgaaaaggtaccatgtgtgctcgctaggcatataatccagatatgctccagagttctgagattcttccatgaaaggttaactagaagctagaatatttttttatatttttgtaacaattggcttttttcatggggggaggggagtagagggttagtatttatagtcctaacaagtccaaaaatttttataagtgtcttcagattataaataaccctccaaattttgcaatgtttacatgttttttttttaagatgacaaatatgcttgatttgctttttaaataaaagtttagctgttctaagagattaacttcaagtaggatggctggttatgatagtttggattttctacaggttctgttgccatgccttttgggtttcagcatcactcgagtcgcagcatgtgggtggggctgtggaaacctggccaggctggacctggtcagccacacctcagagacattgtttccatttggatgtgagcaggcgcaggcctgcatgctctttcctacttagcatcatcagttcttccgcctccttagcatggttctttgtaacagccatgctgggaagctctgaacaataaaatacttccagagtggt